SEQUENCE LISTING (120 sequences)

SEQ ID NO: 1 - protein, 471 aa, Streptococcus pneumoniae
SANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 2 - protein, 476 aa, Artificial
  Streptococcus pneumoniae
ANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWKDQNATKWWRTVYEKTDLPLVRKRTISIWGTTLYPQVEDKVEND

SEQ ID NO: 3 - protein, 476 aa, Artificial
  Streptococcus pneumoniae
ANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWKDQNATKWWRTVYEKTDLPLVRKRTISIWGTTLYPQVEDKVEND

SEQ ID NO: 4 - protein, 476 aa, Artificial
  Streptococcus pneumoniae
ANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWKDQNATKWWRTVYEKTDLPLVRKRTISIWGTTLYPQVEDKVEND

SEQ ID NO: 5 - protein, 476 aa, Artificial
  Streptococcus pneumoniae
ANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIRECTGLAWKDQNATKWWRTVYEKTDLPLVRKRTISIWGTTLYPQVEDKVEND

SEQ ID NO: 6 - protein, 476 aa, Artificial
  Streptococcus pneumoniae
ANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIREATGLAWKDQNATKWWRTVYEKTDLPLVRKRTISIWGTTLYPQVEDKVEND

SEQ ID NO: 7 - protein, 476 aa, Artificial
  Streptococcus pneumoniae
ANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAEKDQNATKWWRTVYEKTDLPLVRKRTISIWGTTLYPQVEDKVEND

SEQ ID NO: 8 - protein, 476 aa, Artificial
  Streptococcus pneumoniae
ANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWKDQNATKWWRTVYEKTDLPLVRKRTISIWGTTEYPQVEDKVEND

SEQ ID NO: 9 - protein, 477 aa, Artificial
  Streptococcus pneumoniae
SANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVEND

SEQ ID NO: 10 - protein, 476 aa, Artificial
  Streptococcus pneumoniae
ANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVEND

SEQ ID NO: 11 - protein, 483 aa, Artificial
  Streptococcus pneumoniae
SANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 12 - protein, 482 aa, Artificial
  Streptococcus pneumoniae
ANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 13 - protein, 19 aa, E. Coli
MKKIWLALAGLVLAFSASA

SEQ ID NO: 14 - protein, 21 aa, E. Coli
MKKTAIAIAVALAGFATVAQA

SEQ ID NO: 15 - protein, 26 aa, E. Coli
MKIKTGARILALSALTTMMFSASALA

SEQ ID NO: 16 - protein, 22 aa, E. Carotova
MKYLLPTAAAGLLLLAAQPAMA

SEQ ID NO: 17 - protein, 23 aa, E. Coli
MSFKKIIKAFVIMAALVSVQAHA

SEQ ID NO: 18 - protein, 28 aa, B. endoxylanase
MFKFKKKFLVGLTAAFMSISMFSATASA

SEQ ID NO: 19 - protein, 19 aa, E. Coli
MIKFLSALILLLVTTAAQA

SEQ ID NO: 20 - protein, 21 aa, E. Coli
MKQALRVAFGFLILWASVLHA

SEQ ID NO: 21 - protein, 504 aa, Artificial
  Streptococcus pneumoniae
MKYLLPTAAAGLLLLAAQPAMAANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 22 - protein, 504 aa, Artificial
  Streptococcus pneumoniae
MKQALRVAFGFLILWASVLHASANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 23 - protein, 13 aa, Artificial
  Streptococcus pneumoniae
  MISC_FEATURE (1)..(1): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (6)..(6): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (6)..(6): Xaa=“Ala” or “Arg” or “Asn” or “Asp” or “Cys” or “Gln” or “Glu” or “Gly” or “His” or “Ile” or “Leu” or “Lys” or “Met” or “Phe” or “Ser” or “Thr” or “Trp” or “Tyr” or “Val” or “Pro”
XTGLAXKDQNATK

SEQ ID NO: 24 - protein, 39 aa, Artificial
  Streptococcus pneumoniae
  MISC_FEATURE (1)..(1): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (6)..(6): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (39)..(39): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (39)..(39): Xaa=“Ala” or “Arg” or “Asn” or “Asp” or “Cys” or “Gln” or “Glu” or “Gly” or “His” or “Ile” or “Leu” or “Lys” or “Met” or “Phe” or “Ser” or “Thr” or “Trp” or “Tyr” or “Val” or “Pro”
XTGLAXKDQNATKWWRTVYEKTDLPLVRKRTISIWGTTX

SEQ ID NO: 25 - protein, 97 aa, Artificial
  Streptococcus pneumoniae
  misc_feature (1)..(1): Xaa can be any naturally occurring amino acid
  MISC_FEATURE (7)..(7): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (12)..(12): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (45)..(45): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (45)..(45): Xaa=“Ala” or “Arg” or “Asn” or “Asp” or “Cys” or “Gln” or “Glu” or “Gly” or “His” or “Ile” or “Leu” or “Lys” or “Met” or “Phe” or “Ser” or “Thr” or “Trp” or “Tyr” or “Val” or “Pro”
  misc_feature (59)..(59): Xaa can be any naturally occurring amino acid
  misc_feature (64)..(64): Xaa can be any naturally occurring amino acid
  misc_feature (97)..(97): Xaa can be any naturally occurring amino acid
XYVAQYYITWNELSYDHQGKEVLTPKAWDRNGQDLTAHFTTSIPLKGNVRNLSVKIREXTGLAXKDQNATKWWRTVYEKTDLPLVRKRTISIWGTTX

SEQ ID NO: 26 - protein, 174 aa, Artificial
  Streptococcus pneumoniae
  MISC_FEATURE (1)..(1): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (78)..(78): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (136)..(136): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (141)..(141): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (174)..(174): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (174)..(174): Xaa=“Ala” or “Arg” or “Asn” or “Asp” or “Cys” or “Gln” or “Glu” or “Gly” or “His” or “Ile” or “Leu” or “Lys” or “Met” or “Phe” or “Ser” or “Thr” or “Trp” or “Tyr” or “Val” or “Pro”
XGDPSSGARVVTGKVDMVEDLIQEGSRFTADHPGLPISYTTSFLRDNVVATFQNSTDYVETKVTAYRNGDLLLDHSGXYVAQYYITWNELSYDHQGKEVLTPKAWDRNGQDLTAHFTTSIPLKGNVRNLSVKIREXTGLAXKDQNATKWWRTVYEKTDLPLVRKRTISIWGTTX

SEQ ID NO: 27 - protein, 402 aa, Artificial
  Streptococcus pneumoniae
  MISC_FEATURE (1)..(1): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  misc_feature (229)..(229): Xaa can be any naturally occurring amino acid
  MISC_FEATURE (238)..(238): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  misc_feature (306)..(306): Xaa can be any naturally occurring amino acid
  MISC_FEATURE (315)..(315): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  misc_feature (364)..(364): Xaa can be any naturally occurring amino acid
  MISC_FEATURE (365)..(365): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  MISC_FEATURE (368)..(368): Xaa=“Ala” or “Arg” or “Asn” or “Asp” or “Asx” or “Cys” or “Gln” or “Glu” or “Glx” or “Gly” or “His” or “Ile” or “Leu” or “Lys” or “Met” or “Phe” or “Ser” or “Thr” or “Trp” or “Tyr” or “Val”
  misc_feature (369)..(369): Xaa can be any naturally occurring amino acid
  MISC_FEATURE (401)..(401): Xaa=“Ala” or “Arg” or “Asn” or “Asp” or “Cys” or “Gln” or “Glu” or “Gly” or “His” or “Ile” or “Leu” or “Lys” or “Met” or “Phe” or “Ser” or “Thr” or “Trp” or “Tyr” or “Val” or “Pro”
  MISC_FEATURE (402)..(402): Xaa=“Ala” or “Arg” or “Asn” or “Asp” or “Cys” or “Gln” or “Glu” or “Gly” or “His” or “Ile” or “Leu” or “Lys” or “Met” or “Phe” or “Ser” or “Thr” or “Trp” or “Tyr” or “Val” or “Pro”
XNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILXGDPSSGARVVTGKVDMVEDLIQEGSRFTADHPGLPISYTTSFLRDNVVATFQNSTDYVETKVTAYRNGDLLLDHSGXYVAQYYITWNELSYDHQGKEVLTPKAWDRNGQDLTAHFTTSIPLKGNVRNLSVKIREXTGLAXKDQNATKWWRTVYEKTDLPLVRKRTISIWGTTX

SEQ ID NO: 28 - protein, 5 aa, Artificial
  Streptococcus pneumoniae
  MISC_FEATURE (1)..(1): Xaa=“Asp” or “Glu”
  MISC_FEATURE (2)..(2): Xaa=“Ala” or “Arg” or “Asn” or “Asp” or “Cys” or “Gln” or “Glu” or “Gly” or “His” or “Ile” or “Leu” or “Lys” or “Met” or “Phe” or “Ser” or “Thr” or “Trp” or “Tyr” or “Val”
  MISC_FEATURE (4)..(4): Xaa=“Ala” or “Arg” or “Asn” or “Asp” or “Cys” or “Gln” or “Glu” or “Gly” or “His” or “Ile” or “Leu” or “Lys” or “Met” or “Phe” or “Ser” or “Thr” or “Trp” or “Tyr” or “Val”
  MISC_FEATURE (5)..(5): Xaa=“Ser” or “Thr”
XXNZX

SEQ ID NO: 29 - protein, 5 aa, Artificial
  Streptococcus pneumoniae
DQNAT

SEQ ID NO: 30 - protein, 7 aa, Artificial
  Streptococcus pneumoniae
  MISC_FEATURE (2)..(2): Xaa=“Asp” or “Glu”
  MISC_FEATURE (3)..(3): Xaa=“Ala” or “Arg” or “Asn” or “Asp” or “Cys” or “Gln” or “Glu” or “Gly” or “His” or “Ile” or “Leu” or “Lys” or “Met” or “Phe” or “Ser” or “Thr” or “Trp” or “Tyr” or “Val”
  MISC_FEATURE (5)..(5): Xaa=“Ala” or “Arg” or “Asn” or “Asp” or “Cys” or “Gln” or “Glu” or “Gly” or “His” or “Ile” or “Leu” or “Lys” or “Met” or “Phe” or “Ser” or “Thr” or “Trp” or “Tyr” or “Val”
  MISC_FEATURE (6)..(6): Xaa=“Ser” or “Thr”
KXXNZXK

SEQ ID NO: 31 - protein, 7 aa, Artificial
  Streptococcus pneumoniae
KDQNATK

SEQ ID NO: 32 - protein, 498 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 33 - protein, 504 aa, Artificial
  Modified pneumolysin
MKYLLPTAAAGLLLLAAQPAMAANKDQNATKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 34 - protein, 504 aa, Artificial
  Modified pneumolysin
MKYLLPTAAAGLLLLAAQPAMAANKAVNDFILAMNYDKKKLLTHKDQNATKGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 35 - protein, 504 aa, Artificial
  Modified pneumolysin
MKYLLPTAAAGLLLLAAQPAMAANKAVNDFILAMNYDKKKLLTHQGEKDQNATKIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 36 - protein, 504 aa, Artificial
  Modified pneumolysin
MKYLLPTAAAGLLLLAAQPAMAANKAVNDFILAMNYDKKKLLTHQGESIKDQNATKNRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 37 - protein, 504 aa, Artificial; identical to SEQ ID NO: 36
  Modified pneumolysin

SEQ ID NO: 38 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASKDQNATKDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 39 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFKDQNATKQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 40 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGKDQNATKVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 41 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVKDQNATKARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 42 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDKDQNATKVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 43 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKDQNATKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 44 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKDQNATKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 45 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDKDQNATKSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 46 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGAKDQNATKVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 47 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTKDQNATKKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 48 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHKDQNATKGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 49 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGKDQNATKAEEWWRTVYEKTDLPLVRKRTISIWGTTEYPQVEDKVENDHHHHHH

SEQ ID NO: 50 - protein, 504 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILVGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 51 - protein, 498 aa, Artificial
  Modified pneumolysin
MKYLLPTAAAGLLLLAAQPAMAANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 52 - protein, 504 aa, Artificial
  Modified pneumolysin
MKYLLPTAAAGLLLLAAQPAMAANKAVNDFILAMNYDKKKLLTHKDQNATKGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 53 - protein, 504 aa, Artificial; identical to SEQ ID NO: 21
  Modified pneumolysin

SEQ ID NO: 54 - protein, 502 aa, Artificial
  Modified pneumolysin
MKKIWLALAGLVLAFSASASANKAVNDFILAMNYDKKKLLTHKDQNATKGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 55 - protein, 502 aa, Artificial
  Modified pneumolysin
MKKIWLALAGLVLAFSASASANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 56 - protein, 502 aa, Artificial
  Modified pneumolysin
MIKFLSALILLLVTTAAQASANKAVNDFILAMNYDKKKLLTHKDQNATKGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 57 - protein, 502 aa, Artificial
  Modified pneumolysin
MIKFLSALILLLVTTAAQASANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 58 - protein, 506 aa, Artificial
  Modified pneumolysin
MSFKKIIKAFVIMAALVSVQAHASANKAVNDFILAMNYDKKKLLTHKDQNATKGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 59 - protein, 506 aa, Artificial
  Modified pneumolysin
MSFKKIIKAFVIMAALVSVQAHASANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 60 - protein, 509 aa, Artificial
  Modified pneumolysin
MKIKTGARILALSALTTMMFSASALASANKAVNDFILAMNYDKKKLLTHKDQNATKGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 61 - protein, 509 aa, Artificial
  Modified pneumolysin
MKIKTGARILALSALTTMMFSASALASANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 62 - protein, 508 aa, Artificial
  Modified pneumolysin
MKMNKKVLLTSTMAASLLSVASVQASANKAVNDFILAMNYDKKKLLTHKDQNATKGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 63 - protein, 508 aa, Artificial
  Modified pneumolysin
MKMNKKVLLTSTMAASLLSVASVQASANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 64 - protein, 511 aa, Artificial
  Modified pneumolysin
MFKFKKKFLVGLTAAFMSISMFSATASASANKAVNDFILAMNYDKKKLLTHKDQNATKGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 65 - protein, 511 aa, Artificial
  Modified pneumolysin
MFKFKKKFLVGLTAAFMSISMFSATASASANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 66 - protein, 504 aa, Artificial
  Modified pneumolysin
MKQALRVAFGFLILWASVLHASANKAVNDFILAMNYDKKKLLTHKDQNATKGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 67 - protein, 504 aa, Artificial; identical to SEQ ID NO: 22
  Modified pneumolysin

SEQ ID NO: 68 - protein, 504 aa, Artificial
  Modified pneumolysin
MKKTAIAIAVALAGFATVAQASANKAVNDFILAMNYDKKKLLTHKDQNATKGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEEWWRTVYEKTDLPLVRKRTISIWGTTEYPQVEDKVENDHHHHHH

SEQ ID NO: 69 - protein, 504 aa, Artificial
  Modified pneumolysin
MKKTAIAIAVALAGFATVAQASANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGEYVAQYYITWNELSYDHQGKEVLTPKAWDRNGQDLTAHFTTSIPLKGNVRNLSVKIREATGLAEKDQNATKWWRTVYEKTDLPLVRKRTISIWGTTEYPQVEDKVENDHHHHHH

SEQ ID NO: 70 - protein, 26 aa, E. Coli
MKMNKKVLLTSTMAASLLSVASVQAS

SEQ ID NO: 71 - protein, 469 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKIMAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFESLIKGVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 72 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALMKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 73 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYIASVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 74 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 75 - protein, 471 aa, Artificial; identical to SEQ ID NO: 74
  Streptococcus pneumoniae

SEQ ID NO: 76 - protein, 471 aa, Artificial; identical to SEQ ID NO: 74
  Streptococcus pneumoniae

SEQ ID NO: 77 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 78 - protein, 471 aa, Artificial; identical to SEQ ID NO: 77
  Streptococcus pneumoniae

SEQ ID NO: 79 - protein, 471 aa, Artificial; identical to SEQ ID NO: 77
  Streptococcus pneumoniae

SEQ ID NO: 80 - protein, 471 aa, Artificial; identical to SEQ ID NO: 77
  Streptococcus pneumoniae

SEQ ID NO: 81 - protein, 471 aa, Artificial; identical to SEQ ID NO: 77
  Streptococcus pneumoniae

SEQ ID NO: 82 - protein, 471 aa, Artificial; identical to SEQ ID NO: 77
  Streptococcus pneumoniae

SEQ ID NO: 83 - protein, 471 aa, Artificial; identical to SEQ ID NO: 77
  Streptococcus pneumoniae

SEQ ID NO: 84 - protein, 471 aa, Artificial; identical to SEQ ID NO: 77
  Streptococcus pneumoniae

SEQ ID NO: 85 - protein, 471 aa, Artificial; identical to SEQ ID NO: 77
  Streptococcus pneumoniae

SEQ ID NO: 86 - protein, 471 aa, Artificial; identical to SEQ ID NO: 77
  Streptococcus pneumoniae

SEQ ID NO: 87 - protein, 471 aa, Artificial; identical to SEQ ID NO: 77
  Streptococcus pneumoniae

SEQ ID NO: 88 - protein, 469 aa, Artificial
  Streptococcus pneumoniae
NKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 89 - protein, 300 aa, E. coli
MTVIAIVVTFNRCALLKKVLHSLLSQSIALNKIIIIDNDSNDDTAKVVHDFSEVDDIFYYYNTGDNLGGAGGFYQGFKIAEQLYYDYLWLMDDDLLPEPDCLEKLIQDRPEGIVQPVRYDLDGACAEISPVEYNLQKIFCRNPKTKTVKEVISTVISDNCREIDIAGVPFEGPLISKSVVNKVGYPNPDFFIFNDDLDYSLRTRSKGFSIKCIVDARATRLLKNNQKNDLKSWKGYFMLRNHYYILRNYGENKLVKNRVYLIMFYYFLKSVFSFDYKFAKVVIFSFKDSFSLKNSKRFRP

SEQ ID NO: 90 - protein, 305 aa, E. coli
MSITNKTIALVIVTYNRCNLLMEMLSSIENMSVKPDIVYVIDNNSSDNTSSVVTECDSRKNINIKYHNTGYNAGGAGGFYIGSKMAYEDGWDRIWLADDDIVLDKECLSNAMEYDDGRTILQPMRYNMDGSCAEISAIQYDLSNPFYLRPKRKTVQNIFNKNILSYDIQSIPFEGPIIPREVFNVIGFPDERFFIFNDDLDFAIRAQRAGFSIKCITNAKIVRKIPFVQSVALKTWKGYFMFRNYFRVQKVYGLSPLIYLRILLVFCLALGHSLVRMDINSIKMLCGALKDGLSQEFKLTEKYKP

SEQ ID NO: 91 - protein, 481 aa, S. pneumoniae
MSRRFKKSRSQKVKRSVNIVLLTIYLLLVCFLLFLIFKYNILAFRYLNLVVTALVLLVALVGLLLIIYKKAEKFTIFLLVFSILVSSVSLFAVQQFVGLTNRLNATSNYSEYSISVAVLADSEIENVTQLTSVTAPTGTNNENIQKLLADIKSSQNTDLTVNQSSSYLAAYKSLIAGETKAIVLNSVFENIIESEYPDYASKIKKIYTKGFTKKVEAPKTSKSQSFNIYVSGIDTYGPISSVSRSDVNILMTVNRDTKKILLTTTPRDAYVPIADGGNNQKDKLTHAGIYGVDSSIHTLENLYGVDINYYVRLNFTSFLKLIDLLGGIDVYNDQEFTAHTNGKYYPAGNVHLDSEQALGFVRERYSLADGDRDRGRHQQKVIVAILQKLTSTEVLKNYSTIINSLQDSIQTNMPLETMINLVNAQLESGGNYKVNSQDLKGTGRMDLPSYAMPDSNLYVMEIDDSSLAVVKAAIQDVMEGR

SEQ ID NO: 92 - protein, 243 aa, S. pneumoniae
MIDIHSHIVFDVDDGPKSREESKALLAESYRQGVRTIVSTSHRRKGMFETPEEKIAENFLQVREIAKEVASDLVIAYGAEIYYTPDVLDKLEKKRIPTLNDSRYALIEFSMNTPYRDIHSALSKILMLGITPVIAHIERYDALENNEKRVRELIDMGCYTQVNSSHVLKPKLFGERYKFMKKRAQYFLEQDLVHVIASDMHNLDGRPPHMAEAYDLVTQKYGEAKAQELFIDNPRKIVMDQLI

SEQ ID NO: 93 - protein, 231 aa, S. pneumoniae
MMKEQNTIEIDVFQLVKSLWKRKLMILIVALVTGAGAFAYSTFIVKPEYTSTTRIYVVNRNQGDKPGLTNQDLQAGTYLVKDYREIILSQDVLEEVVSDLKLDLTPKGLANKIKVTVPVDTRIVSISVNDRVPEEASRIANSLREVAAQKIISITRVSDVTTLEEARPAISPSSPNIKRNTLIGFLAGVIGTSVIVLHLELLDTRVKRPEDIENTLQMTLLGVVPNLGKLK

SEQ ID NO: 94 - protein, 227 aa, S. pneumoniae
MPTLEIAQKKLEFIKKAEEYYNALCTNIQLSGDKLKVISVTSVNPGEGKTTTSINIAWSFARAGYKTLLIDGDTRNSVMLGVFKSREKITGLTEFLSGTADLSHGLCDTNIENLFVVQSGSVSPNPTALLQSKNFNDMIETLRKYFDYIIIDTPPIGIVIDAAIITQKCDASILVTATGEANKRDIQKAKQQLKQTGKLFLGVVLNKLDISVNKYGVYGSYGNYGKK

SEQ ID NO: 95 - protein, 211 aa, S. pneumoniae
MKNGNRIYSWRLFMYGIIKRLGDILLSLIGIIILCPVFMIIAIAIKLDSEGPVIFKQKRFGIHKEYFYILKFRSMKIDAPKNVAPRNLYNPEQWITKVGAFLRKTSLDELPQLFNILVGNMSIVGPRPAGINELDLIAERDKYGANDILPGLTGWAQINGRDTLSVEMKTELDGYYVKHLSLIMDIRCIVKTIPYVLKRKGIVEGSGKKES

SEQ ID NO: 96 - protein, 409 aa, S. pneumoniae
MKILFVCQHYKPEPFRLSDICEDLVRKGHEVSVLAGIPNYPEGKIYADYRHNKKRREIIEGVTIYRSYTIPRKKSVVFRLLNYFSFAISSTLGVLLGRYKTKDGSNFDCVFVNQLSPVMMAWAGMAYKKKYKKPMFLYCMDVWPDSLTVGGVKQDGLIFKLFKFISKKVYRASDYIFVTSPSFKNYFVKQFDISEQKITYLPQYAEDLFIPDESIVNKESVDLTFAGNIGKAQNLETILKAASLIEKNTNLPKKIHFHFVGDGTELLSMKALAHELELKNISFYGRRSLEEMPSFYKKSDAMLVSLIGDSIVSRTIPGKVQSYMAAGKPIIGAISGDAKIIVEEANCGYVSPERDVKQLAKNICKFSMLSIKRQRELGKKARCYYENHFSKEQFMLELETCLERESKKE

SEQ ID NO: 97 - protein, 358 aa, S. pneumoniae
MRVLFILSDNIYLTPYFNFYKELLKKLSISYDVIYWDKNINEIITKQNYYRISFSGKGKLSKILGYVKFRKEIKKKLKENDYDMILPLHSIVSFILVDFLLFSFKNRYIYDIRDYSYEKFLVYRLVQKQLVKNSLMNIVSSDGYKFFLPMGEYFTTHNLPNMIELNEVKQLKNNSTFPIQLSYIGLIRFQEQNKKIIDFFANDSRFQLNFIGTNAGELREFCQEKNISNVNLVDTFQPKDTMSFYKNTDAVLNLYGNHTPLLDYALSNKLYFAALLYKPILVCEDTYMEKVSIENGFGFVLPMKDESEKDCLALYIQNLDRKQLIKNCDNFMDRISLEKQKTEIELEKRILSLRKKND

SEQ ID NO: 98 - protein, 372 aa, S. pneumoniae
MIKVLHLFTTLDSGGVESFLFNYYSHIDRKKIQFDFIVPGKEQGFLEDKMKELGAKVYHVPLLRKKPLHQFLSLARIIKKGDYDIVHCHGYKSAIGLILSKIIGCKIRIIHSHMAYVTENSFQKVLRKLVTILVKILATHWFACGEDSAKWLYGEKAYKDGKIEIIFNAIDLKKYQFLSDVREKCRRELDVSNKFVLGNIARLSDQKNQSYLFNVLKELILIKPNVILLLVGNGEDEQKLKQKALELNLTPYVLFLGRRTDISDLLSAMDVFLLPSKYEGLPVSLVEAQASGLQILSSDTVTQEVDVTKNISYLPINEESVLLWKDKVLSLTSEECNRFEINNSMTDGLYDICYQASKLLNRYQEMCVIKEI

SEQ ID NO: 99 - protein, 466 aa, S. pneumoniae
MQTKYICRVTLVTLSFIFAFCYLFWTLDNWNNGFLISNYVPSIFIWVCFLIIFQITGFILQKVSIYDFSVWYLILSYFFMFGLIFNEYMGFQTTLLWSPSNFYNNEELFHSYIFIIWILFCYSVGYLFFYSDGKVHYHSEVQNYQENEEKILYNAGRILTGVGFISRVITDSKTVLAVRAANSYSAYSEAASSGIIDDLGVLMLPGVFSLFYSDKLSRVIKRTIFWVMLFYLILIMILTGSRKIQVFSILALVLVYTQSLGITFSKKRVLVFLIVTVFLLNVLVVIRGHRFDLNTIGIYLFDSFSSLDFVKNILGEVFSESGLTSLTVASAVTVVPSSIPYEYGMTFLRTILSIFPIGWLVGDFFDKASATVVINKFLGLPVGSSFVEELFWNFGYYGGVFWSFVLGIFSGWRLNFRAFQTSKISKVIYFSVISQLLLLVRSSSIDVYRPIMYSLIMIFIFRRLKK

SEQ ID NO: 100 - protein, 360 aa, S. pneumoniae
MVKKIMLHGATDYGSSNYGDYLYGEIVYDLLESKGYEVSFYNPSDFFQMYLKEYRQKQSFTKKQADAILYIPGGYFGEGHNARFRDNLIQFKRFLPLGIWASYFKKPIGVLGIGAGPNNDSLMNYGIKRIINHAQFITVRDRESFDSLKHLSPSAPVHETFDLIISSKLREEKTEQLCQLKREAKDKKIILVHYNHSKKALEKFAESISLFLENNPNYYVVVTSDSILPYEDAYYQEFRKLVRTEDCFQFKYHSPAEMTSLLKMVDVVLTCKLHVGVVATCFNKSVIAIACHPEKTARYYGAIGELQRCESLFDSSVNSIVKKLETFHLKPITIPSELVLKARSSLDYLDLFLEGLVRES

SEQ ID NO: 101 - protein, 413 aa, S. pneumoniae
MKVDRISFIKNTSSLYILNIVKLLFPLLTLPYLTRVLSLDAYGMVIYVKALIAYVQLVIDFGFMISATKNIVNACTTPSKIGRIVGDTLVEKIFLSIISILIYTILMWQIPIMRENILFSVFYLLATVTNIFIFDFLFRGIEKMHAVAIPYIISKTIITILTFIVVKDDSSILWIPILEGIGNLVAAVVSYRFLHYYGIKLSFSYLSVWVKDLKESSIYFLSNFATTIFGVFTTVISGFYLQSQEIAFWGIAMQLLSAAKSLYNPIANSLYPHMIRTKDIQSVKSINRIMFIPIIFGVLIVLFFSNQILSIIGGEKYTVSADFLKYLLPAFVASFYSMIYGWPVLGAIDKVKETTMTTILASIVQTLGLGIFILSDNFSLVTLAICSSMSEVVLWISRYLIYFKNRSLFVRSK

SEQ ID NO: 102 - protein, 365 aa, S. pneumoniae
MKKVVVVFGTRPEAIKMCPLVKELRTRKNIETLVCVTGQHRQMLDQVLDTFGIIPDFDLSIMKDKQTLFDVTIGILEGMKAILESEKPDLVLVHGDTSTTFASSLAAFYLQIPIGHVEAGLRTYDIYSPYPEEFNRQAVGVLAQYHFTPTQLSKDNLLREGKTPESIFVTGNTAIDALQTTIQEDYTHPELEWIGESRFILITAHRRENLGEPMRHMFRAIRRIIEEYSDVKAIYPIHMNPRVRQIAEEELSGCERIKMIEPLEVLDFHNFLSRSYLILTDSGGIQEEAPSLGKPVLVMRDTTERPEGIEAGTLKLVGADENNIYRHFKELLENDSVYQAMSQASNPYGDGTACKKIADILEGEV

SEQ ID NO: 103 - protein, 351 aa, S. pneumoniae
MSQFTGKTLLITGGTGSFGNAVLKRFLETDVSEIRIFSRDEKKQDDMRHEFQVKVPEVAGKIRFYLGDVRDLASVKNAMHGVDYVFHAAALKQVPSCEFFPVEAVKTNILGTENVLTAAIEAGVKQVICLSTDKAAYPVNAMGTSKAMMEKIAVAKSRTVNPEHTKICVTRYGNVLCSRGSVVPLWIEQIKQGNALTITEPSMTRFVMTLEEAVDLVLFAFEEGKSGDILVQKAPACTIEVLAKAVSEIFASEQDIKIIGIRHGEKRYETLLTNEECANAIDLGDFYRVPSDNRNLNYDKYFKDGSTNRNLLTEFNSNNTDLMDVEQVKRKLLELDEIQTAIRDMVADEEM

SEQ ID NO: 104 - protein, 409 aa, S. pneumoniae
MIKNILITGAKGFVGKNLICTLEALKDGRDRTRPNLEIGEIFQYDRDTDPILLDEYCKKADFVFHLAGVNRPQNPDEFMEGNYGFSSRLLEILEKYENTCPVLLSSSTQASLEGRFSNSIYGQSKLAGEELFFEYGKKTGAPVLVYRFPNLYGKWCRPNYNSAVATFCYNLAHDLPIQVNDPSVELELLYIDDLIQECLTALEGNPHRCNLDGLQILPSPSGNYCYVPTTHRATLGEIVSLLETFKKQPDSLVMPEIPQGSFKKKLYSTYLSYLPVDKFKFPLKMNIDERGSFTELLKTENTGQFSVNISKPGITKGQHWHHSKWEFFMVVSGRALIQERRIGLDENGQEYPILNFEVSGDKIEAIHMIPGYAHNIINLSDTENLITVMWANESFDPRHPDTFFEQVEK

SEQ ID NO: 105 - protein, 394 aa, S. pneumoniae
MKIKTDYSDIHFKDNGKLKLLIIVGTRPEIIRLSSVITKCRKYFDVILAHTGQNYDYNLNGIFFDNLGLDTPDVYMDAVGDDLGATVGNIINTSYKLMNQIKPDALLILGDTNSCLSAIAAKRLHIPIFHMEAGNRCKDECLPEETNRRIVDVISDVNLAYSEHARKYLHECGLPKERTYVTGSPMAEVLHKNLSAIESSDIHERLGLKKGGYILLSAHREENIDTDKNFISLFTAINQLAEKYNMPILYSCHPRSKKRLQESGFKLDKRVIQHEPLGFHDYNCLQMNAFVVVSDSGTLPEESSFFTSQGYPFPAVCIRTSTERPESLDKAGFILAGIDENSLLQAVETAVSLAQDEDFGLPVPDYVEENVSTKVVKIIQSYTGIVDKIVWRKS

SEQ ID NO: 106 - protein, 484 aa, S. pneumoniae
MLIMSRRFKKSRSQKVKRSVNIVLLTIYLLLVCFLLFLIFKYNILAFRYLNLVVTALVLLVALVGLLLIIYKKAEKFTIFLLVFSILVSSVSLFAVQQFVGLTNRLNATSNYSEYSISVAVLADSDIENVTQLTSVTAPTGTDNENIQKLLADIKSSQNTDLTVDQSSSYLAAYKSLIAGETKAIVLNSVFENIIESEYPDYASKIKKIYTKGFTKKVEAPKTSKNQSFNIYVSGIDTYGPISSVSRSDVNILMTVNRDTKKILLTTTPRDAYVPIADGGNNQKDKLTHAGIYGVDSSIHTLENLYGVDINYYVRLNFTSFLKMIDLLGGVDVHNDQEFSALHGKFHFPVGNVHLDSEQALGFVRERYSLADGDRDRGRNQQKVIVAILQKLTSTEALKNYSTIINSLQDSIQTNMPLETMINLVNAQLESGGNYKVNSQDLKGTGRTDLPSYAMPDSNLYVLEIDDSSLAVVKAAIQDVMEGR

SEQ ID NO: 107 - protein, 243 aa, S. pneumoniae
MIDIHSHIVFDVDDGPKSREESKALLAESYRQGVRIIVSTSHRRKGMFETPEEKIAENFLQVREIAKEVASDLVIAYGAEIYYTPDVLDKLEKKRIPTLNDSRYALIEFSMNTPYRDIHSALSKILMLGITPVIAHIERYDALENNEKRVRELIDMGCYTQVNSSHVLKSKLFGERYKFMKKRAQYFLEQDLVHVIASDMHNLDGRPPHMAEAYDLVTQKYGEAKAQELFIDNPRKIVMDQLI

SEQ ID NO: 108 - protein, 231 aa, S. pneumoniae
MMKEQNTIEIDVFQLFKTLWKRKLMILLVALVTGAGAFAYSAFIVKPEYTSTTRIYVVNRDQGDKSGLTNQDLQAGSYLVKDYREIILSQNVLEKVATNLKLDIPAKTLARKVQVTVPVDTRIVSISVKDKQPEEASRIANSLREVAAEKIIAVTRVSDVTTLEEARPATTPSSPNVGRNSLFGFFGGAVVTVIAVLLIELFDIRVKRPEDVEDVLQIPLLGVVPDLDKMK

SEQ ID NO: 109 - protein, 224 aa, S. pneumoniae
MPTLEISQAKLDFVKKAEENYNALCTNLQLSGDDLKVFSITSVKQGEGKSTTSTNIAWAFARAGYKTLLIDGDIRNSVMLGVFKARDKITGLTEFLSGTTDLSQGLCDTNIENLFVIQAGSVSPNPTALLQSKNFSTMLETLRKYFDYIIVDTAPVGVVIDAAIITQKCDASILVTKAGEINRRDIQKAKEQLEHTGKPFLGVVLNKFDTSVDKYGSYGNYGKK

SEQ ID NO: 110 - protein, 346 aa, S. pneumoniae
MKVTIIGQIKNKRTGLGKAINDFRDYCCNRATRVTEIDITNNFNFLSSLFQILISDTDVYYFTPAGSVAGNIRDSLFLFFMIMKRKKIVTHFHNSAFGNVMRQHPTLMIINRILYSKVDLIILLGEKSKIMFQQLRILDEKFKIIRNGVDGYLFIEKNELNKKMSDLPINIIFFSNMIREKGYEILLEVAKKMVGDEKYHFYFSGKFQDNNLKTRFINEIYSMNNVTYLDGVYGSDKKKLLQKMHYFVLPSYYKDETLPISMLEAMANGLYIIVSDVGVVSEVINKETASLIEMINEETADSIIEIINQTSNKLNELDFNVSKYKQELLNENIQASIYQQLERIAN

SEQ ID NO: 111 - protein, 332 aa, S. pneumoniae
MTKKKNTGKILTVVVPSYNAENYLQETMPTILSAKNIERVELLIVNDGSTDRTEEIARQFEREYEGIVRVISKENCGHGSAVNAGIENAVGNYFKVVDADDWVNTNNLEDLIVFLSEVDVDQVLSPYDKIFVNYRGDIEREEECNEFSQVENEVIYSAEEFYTRIKQTVGMHSITVKTSLLQENNIRLSEKMFYVDMEYIVYMLPYVKKVVLFDKSIYRYRLGTETQSISMASYIKNRDMHKQVIYHLVDFYNQMRSSAVLRRITWKLILNLIRQQWIIYFNLSKKEGKNSECFEFDNWLIKEGRIKKIPLYFFKAVKYIRFKVKYFLGIRK

SEQ ID NO: 112 - protein, 322 aa, S. pneumoniae
MRKIGKVINEYFVLRKSFTPAIARNKLFEKFWGRIGNYKIFNNIASNFYQYKHETIINFLEKDFSQFLKSYNFKEVSHKEIEQRKIFSMWIQGYESAPKLVQKTIDSQRKYAEKYGYKFVFLDENNIREYVTLPSEIVEKYENGTIDFIKYSDVVRGTLLSKYGGVWLDSTIYVDSSRELNYLKKDFYTIRAKTHERVPKYIANGRWSAFCLSGEKQNIVFDFLEKFHVAYFMKYDIVLDYFLIDYIIELGYRTNDLIRNYIDKVEENNQELFFLADNFSNQYDEKEWAGVLSTTALFKCSYKCPINEATGTYFDRLMKGEL

SEQ ID NO: 113 - protein, 323 aa, S. pneumoniae
MISVIVPVYNVADYLRFALDSLLEQTYKDFEVILVNDGSTDNSGEICDEYGKLYDNIHVFHKKNGGLSDARNFGLEKSRGEFITFLDSDDYFEPYALELLITIQKKYDVDIVSTKGGITYSHDIYSKKLMAEDYLTVKILTNKEFLAAVYYNDEMTVSAWGKLYKRDLFKTIFPKGKIYEDLYVVAERLLNIKTVAHTDLPIYHYYQRQGSIVNSTFSDRQYDFFDAIDHNEAIIKKFYCGDKELLAALNAKRVIGSFILSNSAFYNSKNDITKIIRIIKPYYWEVIKNKKIPMKRKVQCVLFLLSPNYYYKIKDKMLQRGRI

SEQ ID NO: 114 - protein, 455 aa, S. pneumoniae
MNGKIVKPSLAIIQSFLVILLTYLLSAVREAEIVSTTAIALYILHYFVFYISVYGQDFFKRGYLIELVQTLKYILFFALAISISNFFLEDRFSISRRGMIYFLTLHALLVYVLNLFIKWYWKRTYPNFKGSKKILLLTATFRVEKVLDRLIESNEVVGELVAVSVLDKPDFQHDCLKVVAEGEIVNFATHEVVDEVFINLPSEKYNIGELVSQFETMGIDVTVNLNAFDRSLARNKQIREMAGLNVVTFSTTFYKTSHVIAKRIIDIMGALVGLILCGLVSIVLVPLIRKDGGSAIFAQTRIGKNGRQFTFYKFRSMCVDAEAKKRELMEQNTMQGGMFKVDDDPRITKIGRFIRKTSLDELPQFYNVLKGDMSLVGTRPPTVDEYEHYTPEQKRRLSFKPGVTGLWQVSGRSEIKNFDEVVKLDVAYIDDWTIWKDIEILLKTVKVVLMKDGAK

SEQ ID NO: 115 - protein, 268 aa, S. pneumoniae
MERSRLIDVKIIVATHKEVKMPQDNSLYLPIHVGRDGKSDIGFIGDNTGDNISSLNPYYCELTGLYWAWKNLDYNYLGLVHYRRYFTNKSQGYNENVNMDDLILSRANVEILLEKSDIIVPKKRKYYIETLYSHYAHTLNGEHLDLARKIIEQNSSEYLSSFDKVMKQRSGYMFNMFIMKKELLDDYLPWLFSILDTMYEQMDLTDYTLFESRLFGRVSELLFNVWLCQKGITPKEVPFMYMERVDLFEKGKSFLMAKFFGKKYGQSF

SEQ ID NO: 116 - protein, 226 aa, E. coli
MKRKLVDFCIISLPQHNERRDKLKNEMAKYDIECRVSHAIDGRKLLAEKYFSLFKIRSSKMFGRGFLTPSELGCFLSHKKALTEFLASGRKWLVVLEDDVLPKENVKYLDEMINSFCSSSVYILGGQDGLKSFSRVIMGRKSICGVRKVILGTHRWLYRTCCYCVDIKGAERILRLMEENSFFCDDWSYIVRNAKLDNVFYGQYFSHPVNLNSSSIEAERLFIAEK

SEQ ID NO: 117 - protein, 251 aa, E. coli
MMGLFMGNETVSIIMPAYNAEETIKDSILSILKQTYEDFKLYIINDNSSDSTEHIIKSIIDERIVYLLNRNGKGVSSARNVGIAACNGRYIAFCDSDDVWFETKLEEQLKILSAGNYKVVCSNYEVFYADTNVIKERRFKEVITYNNMLQSNHIGNLTGIYDSTQIGKVYQQEIGHEDYLMWLTIVKKAKLVYCIQKNLARYYIHNTGLSSNKFTAAMWQWNIYRRVLSFSLFKSLVLFFIYSVRALAKRL

SEQ ID NO: 118 - protein, 331 aa, E. coli
MNDNVLLIGASGFVGTRLLETAIADFNIKNLDKQQSHFYPEITQIGDVRDQQALDQALAGFDTVVLLAAEHRDDVSPTSLYYDVNVQGTRNVLAAMEKNGVKNIIFTSSVAVYGLNKHNPDENHPHDPFNHYGKSKWQAEEVLREWYNKAPTERSLTIIRPTVIFGERNRGNVYNLLKQIAGGKFMMVGAGTNYKSMAYVGNIVEFIKYKLKNVAAGYEVYNYVDKPDLNMNQLVAEVEQSLNKKIPSMHLPYPLGMLGGYCFDILSKITGKKYAVSSVRVKKFCATTQFDATKVHSSGFVAPYTLSQGLDRTLQYEFVHAKKDDITFVSE

SEQ ID NO: 119 - protein, 338 aa, E. coli
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 120 - protein, 664 aa, Campylobacter jejuni
IISNDGYAFAEGARDMIAGFHQPNDLSYYGSSLSTLTYWLYKITPFSFESIILYMSTFLSSLVVIPIILLANEYKRPLMGFVAALLASIANSYYNRTMSGYYDTDMLVIVLPMFILFFMVRMILKKDFFSLIALPLFIGIYLWWYPSSYTLNVALIGLFLIYTLIFHRKEKIFYIAVILSSLTLSNIAWFYQSTIIVILFALFALEQKRLNFVIIGILASVTLIFLILSGGVDPILYQLKFYIFRSDESANLTQGFMYFNVNQTIQEVENVDLSEFMRRISGSEIVFLFSLFGFVWLLRKHKSMIMALPILVLGFLALKGGLRFTIYSVPVMALGFGFLLSEFKAILVKKYSQLTSNVCIVFATILTLAPVFIHIYNYKAPTVFSQNEASLLNQLKNIANREDYVVTWWDYGYPVRYYSDVKTLVDGGKHLGKDNFFPSFALSKDEQAAANMARLSVEYTEKSFYAPQNDILKTDILQAMMKDYNQSNVDLFLASLSKPDFKIDTPKTRDIYLYMPARMSLIFSTVASFSFINLDTGVLDKPFTFSTAYPLDVKNGEIYLSNGVVLSDDFRSFKIGDNVVSVNSIVEINSIKQGEYKITPIDDKAQFYIFYLKDSAIPYAQFILMDKTMFNSAYVQMFFLGNYDKNLFDLVINSRDAKVFKLKI

(19) United States
(12) Patent Application Publication
(10) Pub. No.: US 2019/0248841 A1
(43) Pub. Date: Aug. 15, 2019

(54) Immunogenic Composition

(71) Applicant: GlaxoSmithKlíne Biologicals SA, B-1330 Rixensart (BE)

(72) Inventors: Amirreza FARIDMOAYER, Schlieren (CH); Sabina Marietta GERBER, Schlieren (CH)

(21) Appl. No.: 16/308,972

(22) PCT Filed: Jun. 15, 2017
(86) PCT No.: PCT/EP2017/064659

§ 371 Date: Dec. 11, 2018

(30) Foreign Application Priority Data
Jun. 17, 2016 (GB) 1610599.1

(51) Int. Cl. C07K 14/315; A61K 39/09; A61P 31/04

(52) U.S. Cl. CPC C07K 14/3156; A61K 2039/6068; A61P 31/04; A61K 39/092

(57) ABSTRACT

The present invention discloses modified Streptococcus pneumoniae pneumolysin proteins that contain glycosylation site consensus sequences. The invention also discloses a conjugate comprising a modified pneumolysin protein and an antigen (for example a Streptococcus pneumoniae saccharide antigen), wherein the antigen is linked (either directly or through a linker) to an amino acid residue of the modified pneumolysin protein.

### TECHNICAL FIELD

The present invention relates to the field of immunogenic compositions and vaccines, their manufacture and the use of such compositions in medicine. More particularly, it relates to a modified pneumolysin from Streptococcus pneumoniae and its use as a carrier protein. The modified pneumolysin can be used as a carrier protein for other antigens, particularly saccharide antigens or other antigens lacking T cell epitopes, or as an antigen in its own right.

### BACKGROUND

T-independent antigens, for example saccharides, are antigens that elicit antibody production via B lymphocytes without involvement of T-cells. Conjugation of T-independent antigens to carrier proteins has long been established as a way of enabling T-cell help to become part of the immune response for a normally T-independent antigen. In this way, an immune response can be enhanced by allowing the development of immune memory and boostability of the response. Successful conjugate vaccines which have been developed by conjugating bacterial capsular saccharides to carrier proteins are known in the art; the carrier protein having the known effect of turning the T-independent saccharide antigen into a T-dependent antigen capable of triggering an immune memory response. Several carrier proteins are known in the art with tetanus toxoid, diphtheria toxoid, CRM197 and protein D from Haemophilus influenzae being used as carrier protein in commercialised vaccines. CRM197 is currently used in the Streptococcus pneumoniae capsular polysaccharide conjugate vaccine PREVENAR™ (Pfizer) and protein D, tetanus toxoid and diphtheria toxoid are currently used as carriers for capsular polysaccharides in the Streptococcus pneumoniae capsular polysaccharide conjugate vaccine SYNFLORIX™ (GlaxoSmithKline). Other carrier proteins known in the art include EPA (exotoxin A of P. Aeruginosa) for Staphlyococcus aureus serotype 5 and 8 capsular polysaccharides (Wacker et al. J Infect. Dis, 2014 May 15: 209(10):1551-1561) and Outer Membrane Protein (OMP) for Nontypeable Haemophilus influenzae (NTHi) (Wu et al. Infect. Imun. 1999 October 67(1): 5508-5513).

Streptococcus pneumoniae (S. pneumoniae, pneumococcus) is a Gram-positive bacterium responsible for considerable morbidity and mortality (particularly in infants and the elderly), causing invasive diseases such as bacteraemia and meningitis, pneumonia and other non-invasive diseases, such as acute otitis media. About 800,000 children die annually due to pneumococcal disease, especially in emerging countries (O-Brien et al. 2009 Lancet 374:893-902). The increasing number of antibiotic-resistant strains (Linares et al. 2010 Cin. Microbiol. Infect. 16:402-410) and the severity of pneumococcal diseases make vaccination the most effective intervention. The major clinical syndromes caused by S. pneumoniae are widely recognized and discussed in standard medical textbooks (Fedson D S, Muscher D M. In: Plotkin S A, Orenstein W A, editors. Vaccines. 4th edition. Philadelphia WB Saunders Co, 2004a: 529-588). For instance, Invasive Pneumococcal Disease (IPD) is defined as any infection in which S. pneumoniae is isolated from the blood or another normally sterile site (Musher D M. Streptococcus pneumoniae. In Mandell G L, Bennett J E, Dolin R (eds). Principles and Practice of Infectious diseases (5th ed.). New York, Churchill Livingstone, 2001, p 2128-2147).

S. pneumoniae is encapsulated with a covalently linked polysaccharide which confers serotype specificity. There are more than 90 known serotypes of pneumococci, and the capsule is the principle virulence determinant for pneumococci, as the capsule not only protects the inner surface of the bacteria from complement, but is itself poorly immunogenic. Certain serotypes are more abundant than others, to be associated with clinically apparent infections, to cause severe invasive infections and to acquire resistance to one or more classes of antibacterial agents (Rueda, A. M. M. MSc; Serpa, José A. M D; Matloobi, Mahsa M D; Mushtaq, Mahwish M D; Musher, Daniel M. M D. 2010. The spectrum of invasive pneumococcal disease at an adult tertiary care hospital in the early 21st century. Medicine (Baltimore) 89:331-336). According to previous analyses approximately 10 or 11 serotypes account for over 70% of invasive pediatric infections in all regions of the world (Hausdorff W P, Bryant J, Paradiso P R, Siber G R: Which pneumococcal serogroups cause the most invasive disease: implications for conjugate vaccine formulation and use, part I. Clinical infectious diseases: an official publication of the Infectious Diseases Society of America 2000, 30(1):100-121). The distribution of serotypes causing disease varies by age, disease syndrome, disease severity, geographic region, and over time. Pneumococci that are resistant to penicillin, erythromycin, co-trimoxazole or multiple drugs are common in many regions (Evolving trends in Streptococcus pneumoniae resistance: implications for therapy of community-acquired bacterial pneumonia. Jones R N, Jacobs M R, Sader H S. Int J Antimicrob Agents. 2010 September; 36(3):197-204).

Bacterial polysaccharides may elicit a long-lasting immune response in humans if they are coupled to a protein carrier that contains T-cell epitopes. This concept was elaborated almost 100 years ago (Avery, O. T. and W. F. Goebel, 1929, J. Exp. Med. 50:521-533), and proven later for the polysaccharide of Haemophilus influenza type B (HIB) coupled to the protein carrier diphtheria toxin (Anderson, P. 1983, Infect Immun 39:233-8; Schneerson, R. O. Barrera, A. Sutton, and J. B. Robbins. 1980, J Exp Med 152:361-76). This glycoconjugate was also the first conjugated vaccine to be licensed in the USA in 1987 and introduced into the US infant immunization schedule shortly thereafter. Besides HIB, conjugated vaccines have been successfully developed against the encapsulated human pathogens Neisseria meningitidis and S. pneumoniae. After initial licensure of a 7-valent conjugate vaccine containing serotypes 4, 6B, 9V, 14, 18C, 19F, 23F (PCV7), two pneumococcal conjugate vaccines (PCVs) designed to broaden coverage have been licensed. The 10-valent pneumococcal Haemophilus influenzae protein D conjugate vaccine (PCV10) contains serotypes 1, 4, 5, 6B, 7F, 9V, 14 and 23F conjugated to nontypeable H. influenzae protein D, plus serotype 18C conjugated to tetanus toxoid and serotype 19F conjugated to diphtheria toxoid. The 13-valent pneumococcal conjugate vaccine (PCV13) contains the PCV7 (4, 6B, 9V, 14, 18C, 19F, 23F) serotypes plus serotypes 1, 3, 5, 6A, 7F and 19A, conjugated to cross-reactive material CRM197.

Pneumolysin (ply) is a 53 kDa thiol-activated cytolysin found in all strains of S. pneumoniae, which is released on autolysis and contributes to the pathogenesis of S. pneumoniae. It is highly conserved with only a few amino acid substitutions occurring between the ply proteins of different serotypes. Pneumolysin is a multifunctional toxin with a distinct cytolytic (hemolytic) and complement activation activities (Rubins et al., Am. Respi. Cit Care Med, 153:1339-1346 (1996)). The toxin is not secreted by pneumococci, but it is released upon lysis of pneumococci under the influence of autolysin. Its effects include, for example, the stimulation of the production of inflammatory cytokines by human monocytes, the inhibition of the beating of cilia on human respiratory epithelial, the decrease of bactericidal activity and migration of neutrophils, and in the lysis of red blood cells, which involves binding to cholesterol. Expression and cloning of wild-type or native pneumolysin is described in Walker et al. (Infect Immun, 55:1184-1189 (1987)), Mitchell et al. (Biochim Biophys Acta, 1007:67-72 (1989) and Mitchell et al (NAR, 18:4010 (1990)). The structure of pneumolysin, a pore-forming complex, is described in van Pee et al. (Elife. 2017 Mar. 21; 6. pii: e23644. doi: 10.7554/eLife.23644), Lawrence et al. (Sci Rep. 2015 September. 25; 5:14352. doi: 10.1038/srep14352.) and Marshall et al. (Sci Rep. 2015 September. 3; 5:13293. doi: 10.1038/srep13293) and the cytolytic mechanism is described in Park et al. (J Struct Biol. 2016 February; 193(2):132-40. doi: 10.1016/j.jsb.2015.12.002. Epub 2015 Dec. 10) and van Pee et al. (Nano Lett. 2016 Dec. 14; 16(12):7915-7924. Epub 2016 Nov. 3).

Vaccines against pneumococcal disease may be synthesized in vitro by a well-established chemical conjugation technology. Antigenic capsular polysaccharides are extracted from pathogenic organisms, purified, chemically activated and conjugated to a suitable protein carrier. Currently, there are different protein carriers used to produce the glycoconjugates e.g. CRM197 (diphtheria toxoid), tetanus toxoid, and Hemophilus influenzae protein D.

While development of vaccines against such infection is ongoing, there remains a major need for effective vaccines against Streptococcus pneumoniae infection that can safely be produced in high quantities. There is also a need to produce a vaccine with broad serotype coverage against Streptococcus pneumoniae infection.

### SUMMARY OF THE INVENTION

The present invention provides a modified pneumolysin protein and conjugates (including bioconjugates) in which the pneumolysin protein both acts as a carrier protein for a saccharide (e.g. oligosaccharide or polysaccharide) antigen and/or as an antigen in its own right.

Accordingly, there is provided in one aspect of the present invention, a modified pneumolysin protein having an amino acid sequence of SEQ ID NO. 1 or an amino acid sequence at least 80%, 85%, 90%, 92%, 95%, 96%, 97%, 98% or 99% identical to SEQ ID NO. 1, modified in that the amino acid sequence comprises one or more consensus sequence(s) selected from: D/E-X-N-Z-S/T (SEQ ID NO. 28) and K-D/E-X-N-Z-S/T-K (SEQ ID NO. 30), wherein X and Z are independently any amino acid apart from proline. In other words, the amino acid sequence of SEQ ID NO. 1 or an amino acid sequence at least 80%, 85%, 90%, 92%, 95%, 96%, 97%, 98% or 99% identical to SEQ ID NO. 1 is modified so that it comprises one or more consensus sequence(s) selected from: D/E-X-N-Z-S/T (SEQ ID NO. 28) and K-D/E-X-N-Z-S/T-K (SEQ ID NO. 30), wherein X and Z are independently any amino acid apart from proline.

According to a further aspect of the invention, there is provided a conjugate (e.g. bioconjugate) comprising an antigen covalently linked to a modified pneumolysin protein of the invention.

According to a further aspect of the invention, there is provided a polynucleotide encoding a modified pneumolysin protein of the invention.

According to a further aspect of the invention, there is provided a vector comprising a polynucleotide encoding a modified pneumolysin protein of the invention.

According to a further aspect of the invention, there is provided a host cell comprising:

i) one or more nucleic acids that encode glycosyltransferase(s);
ii) a nucleic acid that encodes an oligosaccharyl transferase;
iii) a nucleic acid that encodes a modified pneumolysin protein of the invention; and optionally
iv) a nucleic acid that encodes a polymerase (e.g. wzy).

According to a further aspect of the invention, there is provided a process for producing a bioconjugate that comprises (or consists of) a modified pneumolysin protein linked to a saccharide, said method comprising: (i) culturing the host cell of the invention under conditions suitable for the production of proteins and (ii) isolating the bioconjugate produced by said host cell.

According to a further aspect of the invention, there is provided a bioconjugate produced by a process of the invention, wherein said bioconjugate comprises a saccharide linked to a modified pneumolysin protein.

According to a further aspect of the invention, there is provided an immunogenic composition comprising the modified pneumolysin protein of the invention, or a conjugate of the invention, or a bioconjugate of the invention and a pharmaceutically acceptable excipient or carrier.

According to a further aspect of the invention, there is provided a method of making a immunogenic composition of the invention comprising the step of mixing the modified pneumolysin protein or the conjugate or the bioconjugate with a pharmaceutically acceptable excipient or carrier.

According to a further aspect of the invention, there is provided a method for the treatment or prevention of Streptococcus pneumoniae infection in a subject in need thereof comprising administering to said subject a therapeutically effective amount of a modified pneumolysin protein of the invention, or a conjugate of the invention, or a bioconjugate of the invention.

According to a further aspect of the invention, there is provided a method of immunising a human host against Streptococcus pneumoniae infection comprising administering to the host an immunoprotective dose of a modified pneumolysin protein of the invention, or a conjugate of the invention, or a bioconjugate of the invention.

According to a further aspect of the invention, there is provided a method of inducing an immune response to Streptococcus pneumoniae in a subject, the method comprising administering a therapeutically or prophylactically effective amount of a modified pneumolysin protein of the invention, or a conjugate of the invention, or a bioconjugate of the invention.

According to a further aspect of the invention, there is provided a modified pneumolysin protein of the invention, or a conjugate of the invention, or a bioconjugate of the invention for use in the treatment or prevention of a disease caused by S. pneumoniae infection.

According to a further aspect of the invention, there is provided a modified pneumolysin protein of the invention, or a conjugate of the invention, or a bioconjugate of the invention in the manufacture of a medicament for the treatment or prevention of a disease caused by Streptococcus pneumoniae infection.

### DESCRIPTION OF FIGURES

FIG. 1: Ribbon diagram of the pneumolysin structure from Streptococcus pneumoniae. Domain 1 and 4 are colored in dark gray, domain 2 is in black, and domain 3 is in light gray. The N-terminal loop and the short C-terminal loop are indicated. For the six most successful positions for the introduction of glycosylation sites, amino acids replaced by the -KDQNATK- sequence (SEQ ID NO. 31) are shown as spheres and are labelled.

FIG. 2: In vivo glycosylation of glycoengineered pneumolysin mutants (detoxified pneumolysin, dPly). Shown are immunoblots detecting His-tagged and engineered acceptor protein Ply glycosylated with the capsular polysaccharide from Streptococcus pneumoniae serotype 4 (CP4). Amino acids that were replaced by the -KDQNATK- (SEQ ID NO. 31) glycosylation sequon are indicated above the lanes. Glycosylation results in a mobility shift from the non-glycosylated (Ply_{U}; also referred to as “U-dPLY”) to the glycosylated form of the acceptor protein (Ply_{glyco}; also referred to as “CP4dPLY”). (−) sample represents Ply without any glycosylation site; (+) sample represents glycosylated EPA protein containing two glycosylation sites.

FIGS. 3a-d: Multiple sequence alignment of pneumolysin from 16 different serotypes showing the strong sequence conservation. The alignment was made using Clustal Omega (available at www(.)ebi(.)ac(.)uk).

FIG. 4: Anti-His Westernblot of periplasmic extract from bacterial cultures expressing his-tagged detoxified pneumolysin (dPLY_{His6}).

FIG. 5: Coomassie blue stained SDS-PAGE gel of purified engineered detoxified pneumolysin (dPLY) conjugated to CP4 (see Example 6). Production of CP4 conjugated to engineered dPLY was demonstrated.

FIGS. 6A-E: Studies in guinea pigs demonstrated that CP4-dPLY elicited functional antibodies against both pneumolysin and CP4 (FIGS. 6D, 6E). No significant difference in the level of IgG response and OPA against CP4 was observed between groups injected with the same dose (based on CP) of CP4-dPLY and CP4-EPA (FIGS. 6A, 6D), suggesting that EPA and dPLY are equally efficient carriers for CP4 in guinea pigs. Two different doses 0.1 μg and 1 μg based on glycan were injected into 5-8 week old female Hartley guinea pigs (n=12, Al(OH)₃ only group n=4). Administration of vaccine was intramuscular at days 0, 14 and 28. Shown are the results obtained with sera raised before immunization (d0, pre) and after three immunizations (d42, post).

FIG. 7: Coomassie blue stained SDS-PAGE gel (first panel) and Westernblot (anti-His antibody, second panel; anti-CP33F antibody, third panel) of purified engineered detoxified pneumolysin (dPLY) conjugated to CP33F (see Example 7).

FIG. 8: Westernblot of purified engineered detoxified pneumolysin (dPLY) conjugated to CP12F for plasmid p2401 (Ply_mut48, PellB, pEC415, Kan) in lane 2 and plasmid p2901 (Ply_mut48, TolB, pEC415, Kan) in lane 3 (see Example 12).

### DETAILED DESCRIPTION

### Terminology

Carrier protein: a protein covalently attached to an antigen (e.g. saccharide antigen) to create a conjugate (e.g. bioconjugate). A carrier protein activates T-cell mediated immunity in relation to the antigen to which it is conjugated.

Any amino acid apart from proline (pro, P): refers to an amino acid selected from the group consisting of alanine (ala, A), arginine (arg, R), asparagine (asn, N), aspartic acid (asp, D), cysteine (cys, C), glutamine (gin, Q), glutamic acid (glu, E), glycine (gly, G), histidine (his, H), isoleucine (ile, I), leucine (leu, L), lysine (lys, K), methionine (met, M), phenylalanine (phe, F), serine (ser, S), threonine (thr, T), tryptophan (trp, W), tyrosine (tyr, Y), valine (val, v).

PLY or ply: Pneumolysin from S. pneumoniae

CP: Capsular polysaccharide

LPS: lipopolysaccharide.

wzy: the polysaccharide polymerase gene encoding an enzyme which catalyzes polysaccharide polymerization. The encoded enzyme transfers oligosaccharide units to the non-reducing end forming a glycosidic bond.

waaL: the O antigen ligase gene encoding a membrane bound enzyme. The encoded enzyme transfers undecaprenyl-diphosphate (UPP)-bound O antigen to the lipid A core oligosaccharide, forming lipopolysaccharide.

Und-PP: undecaprenyl pyrophosphate.

Und-P: undecaprenyl phosphate

Reducing end: the reducing end of an oligosaccharide or polysaccharide is the monosaccharide with a free anomeric carbon that is not involved in a glycosidic bond and is thus capable of converting to the open-chain form.

As used herein, the term “bioconjugate” refers to conjugate between a protein (e.g. a carrier protein) and an antigen (e.g. a saccharide) prepared in a host cell background, wherein host cell machinery links the antigen to the protein (e.g. N-links).

As used herein, the term “effective amount,” in the context of administering a therapy (e.g. an immunogenic composition or vaccine of the invention) to a subject refers to the amount of a therapy which has a prophylactic and/or therapeutic effect(s). In certain embodiments, an “effective amount” refers to the amount of a therapy which is sufficient to achieve one, two, three, four, or more of the following effects: (i) reduce or ameliorate the severity of a bacterial infection or symptom associated therewith; (ii) reduce the duration of a bacterial infection or symptom associated therewith; (iii) prevent the progression of a bacterial infection or symptom associated therewith; (iv) cause regression of a bacterial infection or symptom associated therewith; (v) prevent the development or onset of a bacterial infection, or symptom associated therewith; (vi) prevent the recurrence of a bacterial infection or symptom associated therewith; (vii) reduce organ failure associated with a bacterial infection; (viii) reduce hospitalization of a subject having a bacterial infection; (ix) reduce hospitalization length of a subject having a bacterial infection; (x) increase the survival of a subject with a bacterial infection; (xi) eliminate a bacterial infection in a subject; (xii) inhibit or reduce a bacterial replication in a subject; and/or (xiii) enhance or improve the prophylactic or therapeutic effect(s) of another therapy.

As used herein, the term “subject” refers to an animal, in particular a mammal such as a primate (e.g. human).

As used herein, the term “donor oligosaccharide or polysaccharide” refers to an oligosaccharide or polysaccharide from which a oligosaccharide or polysaccharide is derived. Donor oligosaccharides and polysaccharides, as used herein, comprise a hexose monosaccharide (e.g. glucose) at the reducing end of the first repeat unit. Use of the term donor oligosaccharide or polysaccharide is not meant to suggest that an oligosaccharide or polysaccharide is modified in situ. Rather, use of the term donor oligosaccharide or polysaccharide is meant to refer to an oligosaccharide or polysaccharide that, in its wild-type state, is a weak substrate for oligosaccharyl transferase (e.g. PglB) activity or is not a substrate for oligosaccharyl transferase (e.g. PglB) activity. Exemplary donor oligosaccharides or polysaccharides include those from bacteria, including S. pneumoniae CP1, CP2, CP3, CP4, CP5, CP6(A,B,C,D), CP7(A,B,C), CP8, CP9(A,L,N,V), CP10(A,B,C,F), CP11(A,B,C,D,F), CP12(A,B,F), CP13, CP14, CP15(A,B,C,F), CP16 (A,F), CP17 (A,F), CP18(A,B,C,F), CP19(A,B,C,F), CP20, CP21, CP22(A,F), CP23(A,B,F), CP24(A,B,F), CP25(A,F), CP 26, CP27, CP28(A,F), CP29, CP31, CP32(A,F), CP33(A,B,C,D,F), CP34, CP35(A,B,C,D,F), CP36, CP37, CP38, CP39, CP40, CP41(A,F), CP42, CP43, CP44, CP45, CP46, CP47(A,F), and CP48. Those of skill in the art will readily be able determine whether an oligosaccharide or polysaccharide comprises a hexose monosaccharide (e.g. glucose) at the reducing end of the first repeat unit, and thus whether such an oligosaccharide or polysaccharide is a donor oligosaccharide or polysaccharide as encompassed herein.

As used herein, the term “hexose monosaccharide derivative” refers to a derivative of a hexose monosaccharide that can be a substrate for oligosaccharyl transferase activity. In general, hexose monosaccharide derivatives comprise a monosaccharide comprising an acetamido group at position 2. Exemplary hexose monosaccharide derivatives include GlcNAc, HexNAc, deoxy HexNAc, or 2,4-diacetamido-2,4,6-trideoxyhexose.

As used herein, the term “hybrid oligosaccharide or polysaccharide” refers to an engineered oligosaccharide or polysaccharide that does not comprise a hexose at the reducing end of the first repeat unit, but instead comprises a hexose monosaccharide derivative at the reducing end of the first repeat unit.

As used herein, the term “immunogenic fragment” is a portion of an antigen smaller than the whole, that is capable of eliciting a humoral and/or cellular immune response in a host animal, e.g. human, specific for that fragment. Fragments of a protein can be produced using techniques known in the art, e.g. recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Typically, fragments comprise at least 10, 20, 30, 40 or 50 contiguous amino acids of the full length sequence. Fragments may be readily modified by adding or removing 1, 2, 3, 4, 5, 6, 7, 8, 9, 10, 20, 30, 40 or 50 amino acids from either or both of the N and C termini.

As used herein, the term “conservative amino acid substitution” involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a parental polypeptide.

As used herein, the term “deletion” is the removal of one or more amino acid residues from the protein sequence. Typically, no more than about from 1 to 6 residues (e.g. 1 to 4 residues) are deleted at any one site within the protein molecule.

As used herein, the term “insertion” is the addition of one or more non-native amino acid residues in the protein sequence. Typically, no more than about from 1 to 10 residues, (e.g. 1 to 7 residues, 1 to 6 residues, or 1 to 4 residues) are inserted at any one site within the protein molecule.

### Proteins

Pneumolysin (ply or Ply) is a 53 kDa thiol-activated cytolysin found in all strains of S. pneumoniae, which is released on autolysis and contributes to the pathogenesis of S. pneumoniae. Expression and cloning of wild-type or native pneumolysin is described in Walker et al. (Infect Immun, 55:1184-1189 (1987)), Mitchell et al. (Biochim Biophys Acta, 1007:67-72 (1989) and Mitchell et al. (NAR, 18:4010 (1990)). It is highly conserved with only a few amino acid substitutions occurring between the ply proteins of different serotypes. According to Lawrence et al. Sci. Rep. 5, 14352 (2015), ply contains 11% helix and 32% beta-sheet. It is composed of four domains: domain 1 (D1; residues 1 to 21, 58 to 147, 198 to 243 and 319 to 342) consists of three α-helices and one β-sheet, domain 2 (D2; residues 22 to 57 and 343 to 359) contains one β-sheet, domain 3 (D3; residues 148 to 197 and 244 to 318) is composed of a 5-stranded anti-parallel β-sheet that is surrounded by the two α-helical bundles that become transmembrane hairpins TMH1 (residues 160 to 186) and TMH2 (residues 257 to 280), and domain 4 (D4; residues 360 to 470) is folded into a compact β-sandwich. Domain 2 is connected to domain 4 by a single glycine linker. The Trp-rich loop in domain 4 between residues 427 and 437 is conserved across the CDCs and represents the longest stretch of sequence identity (Rossjohn et al. 1998, J. Mol. Biol, 284:1223-1237).

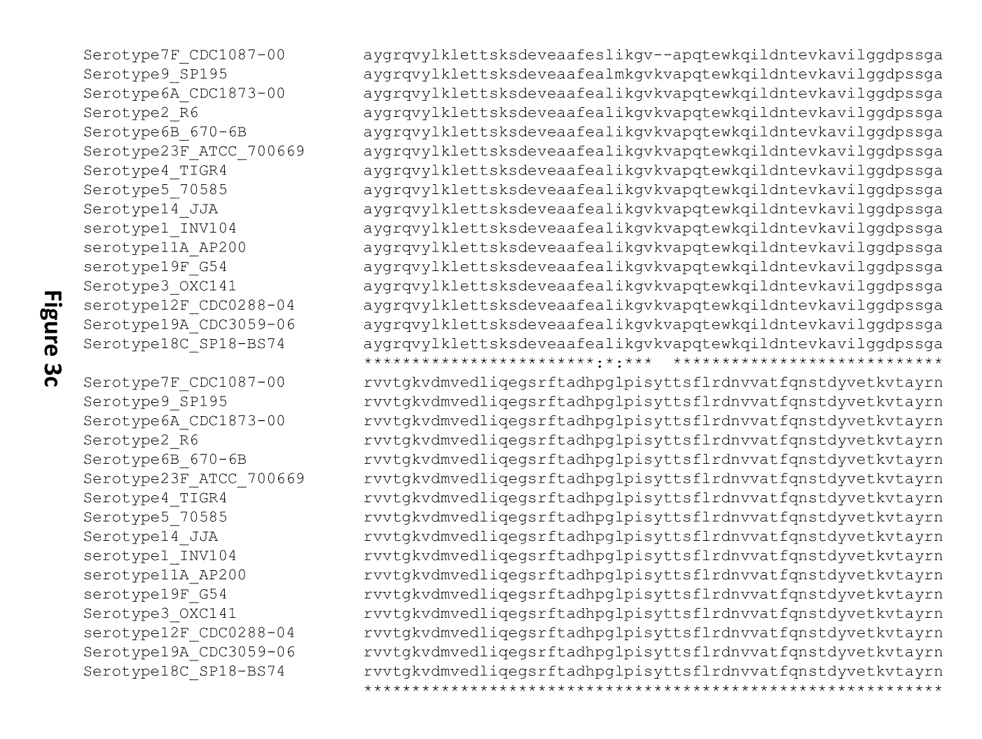

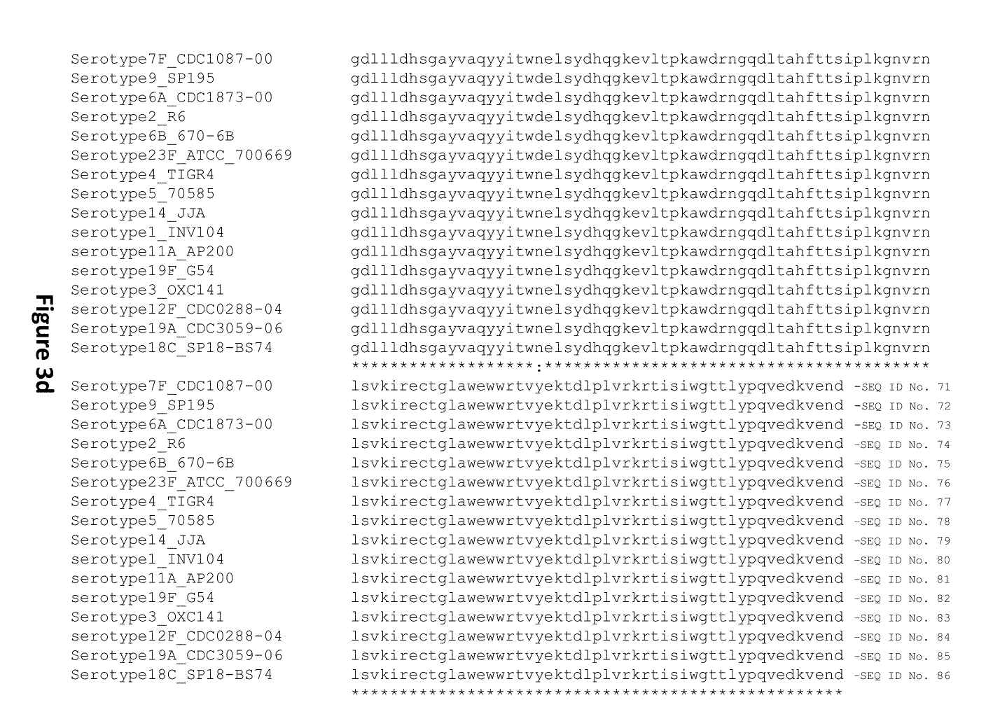

The present invention provides a modified pneumolysin protein comprising (or consisting of) an amino acid sequence of SEQ ID NO. 1 or an amino acid sequence at least 80%, 85%, 90%, 91%, 92%, 93%, 94%, 95%, 96%, 97%, 98% or 99% identical to SEQ ID NO. 1 (e.g. SEQ ID NO. 88), modified in that the amino acid sequence comprises one or more consensus sequence(s) selected from: D/E-X-N-Z-S/T (SEQ ID NO. 28) and K-D/E-X-N-Z-S/T-K (SEQ ID NO. 30), wherein X and Z are independently any amino acid apart from proline. FIGS. 3A-3D provide the sequences (SEQ ID NOs. 71-86) of sixteen wild-type pneumolysin proteins from various S. pneumoniae serotypes, and any one of these proteins (in addition to the wild-type pneumolysin protein SEQ ID NO. 87) may be modified according to the invention so that the amino acid sequence comprises one or more consensus sequence(s) selected from D/E-X-N-Z-S/T (SEQ ID NO. 28) and K-D/E-X-N-Z-S/T-K (SEQ ID NO. 30), wherein X and Z are independently any amino acid apart from proline. These sequences may be modified by the removal of the N-terminal methionine and optionally substitution of the N-terminal methionine for an N-terminal serine for cloning purposes. The sequences may further be modified to contain detoxifying mutations, such as any one or all of the detoxifying mutations described herein.

In an embodiment, the modified pneumolysin protein of the invention may be derived from an amino acid sequence at least 80%, 85%, 90%, 92%, 95%, 96%, 97%, 98% or 99% identical to SEQ ID NO. 1 which is an immunogenic fragment and/or a variant of SEQ ID NO. 1 (e.g. SEQ ID NO. 88).

In an embodiment, the modified pneumolysin protein of the invention may be derived from an immunogenic fragment of SEQ ID NO. 1 comprising at least about 15, at least about 20, at least about 40, or at least about 60 contiguous amino acid residues of the full length sequence, wherein said polypeptide is capable of eliciting an immune response specific for said amino acid sequence. Native pneumolysin is known to consist of four major structural domains (Rossjohn et al. Cell. 1997 May 30; 89(5):685-92). These domains may be modified by removing and/or modifying one or more of these domains. In an embodiment, the fragment of SEQ ID NO. 1 contains exactly or at least 1, 2 or 3 domains. In another embodiment, the fragment of SEQ ID NO. 1 contains exactly or at least 2 or 3 domains. In another embodiment, the fragment of SEQ ID NO. 1 contains at least 3 domains. In an embodiment, the fragment of SEQ ID NO. 1 may comprise (or consist of) the amino acid residues of D1 (residues 1 to 21, 58 to 147, 198 to 243 and 319 to 342) of SEQ ID NO. 1. In another aspect, the fragment of SEQ ID NO. 1 may comprise (or consist of) the amino acid residues of D2 (residues 22 to 57 and 343 to 359) of SEQ ID NO. 1. In another aspect, the fragment of SEQ ID NO. 1 may comprise (or consist of) the amino acid residues of D3 (residues 148 to 197 and 244 to 318) of SEQ ID NO. 1. In another aspect, the fragment of SEQ ID NO. 1 may comprise (or consist of) the amino acid residues of D4 (residues 360 to 470) of SEQ ID NO. 1. For example, the fragment of SEQ ID NO.1 may comprise (or consist of) (i) the amino acid residues 1-342 of SEQ ID NO. 1, (ii) the amino acid residues 22 to 359 of SEQ ID NO. 1, or (iii) the amino acid residues 360-470 of SEQ ID NO. 1.

In an embodiment, the modified pneumolysin protein of the invention may be derived from an amino acid sequence at least 80%, 85%, 90%, 92%, 95%, 96%, 97%, 98% or 99% identical to SEQ ID NO. 1 which is a variant of SEQ ID NO. 1 which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, the modified pneumolysin protein of the present invention may be derived from a variant in which 1 to 10, 5 to 10, 1 to 5, 1 to 3, 1 to 2 or 1 amino acids are substituted, deleted, or added in any combination. For example, the modified pneumolysin protein of the invention may be derived from an amino acid sequence which is a variant of SEQ ID NO. 1 in that it lacks the N-terminal serine (SEQ ID NO. 88).

In an embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated on the basis of the method described by Jameson and Wolf (CABIOS 4:181-186 [1988]).

The term “modified pneumolysin protein” refers to a pneumolysin amino acid sequence (for example, having a pneumolysin amino acid sequence of SEQ ID NO. 1 or an amino acid sequence at least 80%, 85%, 90%, 91%, 92%, 93%, 94%, 95%, 96%, 97%, 98% or 99% identical to SEQ ID NO. 1), which pneumolysin amino acid sequence may be a wild-type pneumolysin amino acid sequence (for example, a wild-type amino acid sequence selected from SEQ ID NOs. 71-87, e.g. SEQ ID NO. 87), which has been modified by the addition, substitution or deletion of one or more amino acids (for example, by addition of a consensus sequence(s) selected from D/E-X-N-Z-S/T (SEQ ID NO. 28) and K-D/E-X-N-Z-S/T-K (SEQ ID NO. 30; or by substitution of one or more amino acids by a consensus sequence(s) selected from D/E-X-N-Z-S/T (SEQ ID NO. 28) and K-D/E-X-N-Z-S/T-K (SEQ ID NO. 30)). The modified pneumolysin protein may also comprise further modifications (additions, substitutions, deletions) as well as the addition or substitution of one or more consensus sequence(s). For example, the N-terminal methionine from a wild-type pneumolysin protein may be removed (or optionally substituted for an N-terminal serine). A signal sequence and/or peptide tag may be added. In an embodiment, the modified pneumolysin protein of the invention may be a non-naturally occurring pneumolysin protein.

In an embodiment of the invention, one or more amino acids (e.g. 1-7 amino acids, e.g. one amino acid) of the pneumolysin amino acid sequence (for example, having an amino acid sequence of SEQ ID NO. 1 or a pneumolysin amino acid sequence at least 80%, 85%, 90%, 91%, 92%, 93%, 94%, 95%, 96%, 97%, 98% or 99% identical to SEQ ID NO. 1, e.g. SEQ ID NO. 88) have been substituted by a five amino acid D/E-X-N-Z-S/T (SEQ ID NO. 28) or by a seven amino acid K-D/E-X-N-Z-S/T-K (SEQ ID NO. 30) (e.g. K-D-Q-N-A-T-K (SEQ ID NO. 31) also referred to as “KDQNATK”) consensus sequence. For example, a single amino acid in the pneumolysin amino acid sequence (e.g. SEQ ID NO. 1) may be replaced with a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence. Alternatively, 2, 3, 4, 5, 6 or 7 amino acids in the pneumolysin amino acid sequence (e.g. SEQ ID NO. 1 or a pneumolysin amino acid sequence at least 80%, 85%, 90%, 92%, 95%, 96%, 97%, 98% or 99% identical to SEQ ID NO. 1) may be replaced with a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence.

Introduction of a consensus sequence(s) selected from: a five amino acid consensus sequence D/E-X-N-Z-S/T (SEQ ID NO. 28) and a seven amino acid consensus sequence K-D/E-X-N-Z-S/T-K (SEQ ID NO. 30) enables the modified pneumolysin protein to be glycosylated. Thus, the present invention also provides a modified pneumolysin protein of the invention wherein the modified pneumolysin protein is glycosylated. In specific embodiments, the consensus sequences are introduced into specific regions of the pneumolysin amino acid sequence, e.g. surface structures of the protein, at the N or C termini of the protein, and/or in loops that are stabilized by disulfide bridges at the base of the protein. In an aspect of the invention, the position of the consensus sequence(s) provides improved glycosylation, for example increased yield. In an embodiment, the consensus sequence(s) selected from D/E-X-N-Z-S/T (SEQ ID NO. 28) and K-D/E-X-N-Z-S/T-K (SEQ ID NO. 30) (e.g. K-D-Q-N-A-T-K (SEQ ID NO. 31)) is located in the modified pneumolysin amino acid sequence at a position within the long N terminal surface loop (A₁₂ to K₃₄) or the short C terminal loop (E₄₂₇ to V₄₃₉) with reference to SEQ ID NO. 1.

In an embodiment, a consensus sequence selected from D/E-X-N-Z-S/T (SEQ ID NO. 28) and K-D/E-X-N-Z-S/T-K (SEQ ID NO. 30) (e.g. K-D-Q-N-A-T-K (SEQ ID NO. 31)) has been added or substituted for one or more amino acids residues 22-57 (e.g. in place of one or more amino acid residue(s) 24-29, or in place of amino acid residues 24, 27 or 29) of SEQ ID NO. 1 or in an equivalent position in an amino acid sequence at least 80%, 85%, 90%, 92%, 95%, 96%, 97%, 98% or 99% identical to SEQ ID NO. 1 (e.g. in an equivalent position in the amino acid sequence of SEQ ID NO. 88). In one aspect, a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for one or more amino acid residue(s) between amino acids 22-57 of SEQ ID NO. 1 or in an equivalent position in an amino acid sequence at least 80%, 85%, 90%, 92%, 95%, 96%, 97%, 98% or 99% identical to SEQ ID NO. 1. In another aspect, a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for one or more amino acids residues between amino acids 24-29 of SEQ ID NO. 1 or in an equivalent position in an amino acid sequence at least 80%, 85%, 90%, 92%, 95%, 96%, 97%, 98% or 99% identical to SEQ ID NO. 1. In another aspect, a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been substituted for an amino acid residue 24, 27 or 29 (e.g. Q₂₄, S₂₇ or E₂₉) of SEQ ID NO. 1 or in an equivalent position in an amino acid sequence at least 80%, 85%, 90%, 92%, 95%, 96%, 97%, 98% or 99% identical to SEQ ID NO. 1.

In an embodiment, a consensus sequence selected from D/E-X-N-Z-S/T (SEQ ID NO. 28) and K-D/E-X-N-Z-S/T-K (SEQ ID NO. 30) (e.g. K-D-Q-N-A-T-K (SEQ ID NO. 31)) has been added or substituted for one or more amino acids at a position between amino acid residues 360-470 (e.g. in place of one or more amino acid residue(s) 427-437, in place of one or more amino acid residue(s) 431-434, or in place of amino acid residues 431 or 434) of SEQ ID NO. 1 or in an equivalent position in an amino acid sequence at least 80%, 85%, 90%, 92%, 95%, 96%, 97%, 98% or 99% identical to SEQ ID NO. 1 (e.g. in an equivalent position in the amino acid sequence of SEQ ID NO. 88). In one aspect, a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for one or more amino acid residue(s) amino acid residue between amino acids 360-470 of SEQ ID NO. 1 or in an equivalent position in an amino acid sequence at least 80%, 85%, 90%, 92%, 95%, 96%, 97%, 98% or 99% identical to SEQ ID NO. 1. In another aspect, a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for one or more amino acids residues between amino acids 427-437 of SEQ ID NO. 1 or in an equivalent position in an amino acid sequence at least 80%, 85%, 90%, 92%, 95%, 96%, 97%, 98% or 99% identical to SEQ ID NO. 1. In another aspect, a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for one or more amino acid residue(s) between amino acids 431-434 of SEQ ID NO. 1 or in an equivalent position in an amino acid sequence at least 80%, 85%, 90%, 92%, 95%, 96%, 97%, 98% or 99% identical to SEQ ID NO. 1. In another aspect, the D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been substituted for amino acid residue 431 or 434 of SEQ ID NO. 1 (i.e. L₄₃₁ or E₄₃₄) or in an equivalent position in an amino acid sequence at least 80%, 85%, 90%, 92%, 95%, 96%, 97%, 98% or 99% identical to SEQ ID NO. 1. In another aspect, the K-D/E-X-N-Z-S/T-K (SEQ ID NO. 30) (e.g. K-D-Q-N-A-T-K (SEQ ID NO. 31)) consensus sequence has been substituted for amino acid residue 434 of SEQ ID NO. 1 (i.e. E₄₃₄) or in an equivalent position in an amino acid sequence at least 80%, 85%, 90%, 92%, 95%, 96%, 97%, 98% or 99% identical to SEQ ID NO. 1.

In an embodiment, a peptide comprising a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for one or more amino acids at a position between amino acid residues 24-29 or amino acid residues 427-437 of SEQ ID NO. 1 or in an equivalent position in an amino acid sequence at least 80%, 85%, 90%, 92%, 95%, 96%, 97%, 98% or 99% identical to SEQ ID NO. 1 (e.g. in an equivalent position in the amino acid sequence of SEQ ID NO. 88). For example, a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for an amino acid residue between amino acids 24-29 or amino acids 427-437 of SEQ ID NO. 1 or in an equivalent position in an amino acid sequence at least 80%, 85%, 90%, 92%, 95%, 96%, 97%, 98% or 99% identical to SEQ ID NO. 1. Thus, the present invention provides a modified pneumolysin protein having an amino acid sequence at least 80%, 85%, 90%, 92%, 95%, 96%, 97%, 98% or 99% identical to SEQ ID NO. 1, said amino acid sequence comprising a consensus sequence selected from: D/E-X-N-Z-S/T (SEQ ID NO. 28) and K-D/E-X-N-Z-S/T-K (SEQ ID NO. 30), wherein X and Z are independently any amino acid apart from proline, wherein the start of said consensus sequence is located between amino acids 24-29 or amino acids 427-437 of SEQ ID NO. 1 or in an equivalent position in an amino acid sequence at least 80%, 85%, 90%, 92%, 95%, 96%, 97%, 98% or 99% identical to SEQ ID NO. 1.

In an embodiment, a peptide comprising a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for one or more amino acids at a position between amino acid residues 24-29 or amino acids 431-434 of SEQ ID NO. 1 or in an equivalent position in an amino acid sequence at least 80%, 85%, 90%, 92%, 95%, 96%, 97%, 98% or 99% identical to SEQ ID NO. 1 (e.g. in an equivalent position in the amino acid sequence of SEQ ID NO. 88). For example, a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for an amino acid residue between amino acids 24-29 or amino acids 431-434 of SEQ ID NO. 1 or in an equivalent position in an amino acid sequence at least 80%, 85%, 90%, 92%, 95%, 96%, 97%, 98% or 99% identical to SEQ ID NO. 1. Thus, the present invention provides a modified pneumolysin protein having an amino acid sequence at least 80%, 85%, 90%, 92%, 95%, 96%, 97%, 98% or 99% identical to SEQ ID NO. 1, said amino acid sequence comprising a consensus sequence selected from: D/E-X-N-Z-S/T (SEQ ID NO. 28) and K-D/E-X-N-Z-S/T-K (SEQ ID NO. 30), wherein X and Z are independently any amino acid apart from proline, wherein the start of said consensus sequence is located between amino acids 24-29 or amino acids 431-434 of SEQ ID NO. 1 or in an equivalent position in an amino acid sequence at least 80%, 85%, 90%, 92%, 95%, 96%, 97%, 98% or 99% identical to SEQ ID NO. 1.

In an embodiment, a peptide comprising a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for one or more amino acids at a position between amino acid residue 24, 27, 29, 431, or 434 (e.g. Q₂₄, S₂₇, E₂₉, L₄₃₁ or E₄₃₄) of SEQ ID NO. 1 or in an equivalent position in an amino acid sequence at least 80%, 85%, 90%, 92%, 95%, 96%, 97%, 98% or 99% identical to SEQ ID NO. 1 (e.g. in an equivalent position in the amino acid sequence of SEQ ID NO. 88). In an embodiment, the consensus sequence K-D/E-X-N-Z-S/T-K (SEQ ID NO. 30) (e.g. K-D-Q-N-A-T-K (SEQ ID NO. 31)) has been added or substituted for amino acid residue E₄₃₄ in SEQ ID NO. 1 or in an equivalent position in an amino acid sequence at least 80%, 85%, 90%, 92%, 95%, 96%, 97%, 98% or 99% identical to SEQ ID NO. 1. For example, a D/E-X-N-Z-S/T (SEQ ID NO. 28) or K-D/E-X-N-Z-S/T-K (SEQ ID NO. 30) (e.g. K-D-Q-N-A-T-K (SEQ ID NO. 31)) consensus sequence has been added or substituted for an amino acid residue 24, 27, 29, 431, or 434 of SEQ ID NO. 1 (i.e. Q₂₄, S₂₇, E₂₉, L₄₃₁ or E₄₃₄) or in an equivalent position in an amino acid sequence at least 80%, 85%, 90%, 92%, 95%, 96%, 97%, 98% or 99% identical to SEQ ID NO. 1. Thus, the present invention provides a modified pneumolysin protein having an amino acid sequence at least 80%, 85%, 90%, 92%, 95%, 96%, 97%, 98% or 99% identical to SEQ ID NO. 1, said amino acid sequence comprising a consensus sequence selected from: D/E-X-N-Z-S/T (SEQ ID NO. 28) and K-D/E-X-N-Z-S/T-K (SEQ ID NO. 30), wherein X and Z are independently any amino acid apart from proline, wherein the start of said consensus sequence is located at amino acids 24, 27, 29, 431, or 434 of SEQ ID NO. 1 or in an equivalent position in an amino acid sequence at least 80%, 85%, 90%, 92%, 95%, 96%, 97%, 98% or 99% identical to SEQ ID NO. 1. It will be understood by a person skilled in the art, that reference to “between amino acids . . . ” (for example “between amino acids 427-437”) is referring to the amino acid number counting consecutively from the N-terminus of the amino acid sequence, for example “between amino acids 427-437 . . . of SEQ ID NO. 1” refers to position in the amino acid sequence between the 427^{th} and 437^{th} amino acid of SEQ ID NO. 1 including both the 427^{th} and 437^{th} amino acid. Thus, in an embodiment where “a consensus sequence selected from D/E-X-N-Z-S/T (SEQ ID NO. 28) and K-D/E-X-N-Z-S/T-K (SEQ ID NO. 30) (e.g. K-D-Q-N-A-T-K (SEQ ID NO. 31)) has been added at or substituted for one or more amino acids between amino acid residues 427-437”, the consensus sequence may have been added at or substituted for any one (or more) of amino acid numbers 427, 428, 429, 430, 431, 432, 433, 434, 435, 436 or 437 in SEQ ID NO. 1. A person skilled in the art will understand that when the pneumolysin amino acid sequence is a variant and/or fragment of an amino acid sequence of SEQ ID NO. 1, such as an amino acid sequence at least 80%, 85%, 90%, 92%, 95%, 96%, 97%, 98% or 99% identical to SEQ ID NO. 1, the reference to “between amino acids . . . ” refers to a the position that would be equivalent to the defined position, if this sequence was lined up with an amino acid sequence of SEQ ID NO. 1 in order to maximise the sequence identity between the two sequences (Sequence alignment tools are not limited to Clustal Omega (www(.)ebi(.)ac(.)ac(.)uk) MUSCLE (www(.)ebi(.)ac(.)uk), or T-coffee (www(.)tcoffee(.)org). In one aspect, the sequence alignment tool used is Clustal Omega (www(.)ebi(.)ac(.)ac(.)uk).

The addition or deletion of amino acids from the variant and/or fragment of SEQ ID NO.1 could lead to a difference in the actual amino acid position of the consensus sequence in the mutated sequence, however, by lining the mutated sequence up with the reference sequence, the amino acid in an equivalent position to the corresponding amino acid in the reference sequence can be identified and hence the appropriate position for addition or substitution of the consensus sequence can be established. For example, FIGS. 3A-D show a sequence alignment of pneumolysins from S. pneumoniae sequences from different serotypes: Serotype6A_CDC1873-00, Serotype9_SP195, Serotype2_R6, Serotype6B_670-6B, Serotype23F_ATCC_700669, Serotype4_TIGR4, Serotype5_70585, Serotype14_JJA, Serotype11A_AP200, Serotype19F_G54, Serotype3_OXC141, Serotype12F_CDC0288-04, Serotype18C_SP18-BS74, Serotype19A_CDC3059-06, Serotype1_INV104, Serotype7F_CDC1087-00.

In an embodiment, the modified protein of the invention comprises at least 1, 2, 3 or 4 D/E-X-N-X-S/T consensus sequences or exactly 1, 2, 3, 4, 5, or 6 D/E-X-N-X-S/T consensus sequences. In an embodiment, the modified protein of the invention comprises at least 1, 2, 3 or 4 D/E-X-N-Z-S/T (SEQ ID NO. 28) consensus sequences or exactly 1, 2, 3, 4, 5, or 6 D/E-X-N-Z-S/T (SEQ ID NO. 28) consensus sequences. In an embodiment, the modified protein of the invention comprises at least 1, 2, 3 or 4 K-D/E-X-N-Z-S/T-K (SEQ ID NO. 30) consensus sequences or exactly 1, 2, 3, 4, 5, or 6 K-D/E-X-N-Z-S/T-K (SEQ ID NO. 30) consensus sequences. In an embodiment, the modified protein of the invention comprises a single consensus sequence selected from D/E-X-N-Z-S/T (SEQ ID NO. 28) and K-D/E-X-N-Z-S/T-K (SEQ ID NO. 30). In an embodiment, the consensus sequence is D/E-X-N-Z-S/T (SEQ ID NO. 28), wherein X is Q (glutamine) and Z is A (alanine), e.g. D-Q-N-A-T (SEQ ID NO. 29) also referred to as “DQNAT”. In an embodiment, the consensus sequence is K-D/E-X-N-Z-S/T-K (SEQ ID NO. 30), wherein X is Q (glutamine) and Z is A (alanine), e.g. K-D-Q-N-A-T-K (SEQ ID NO. 31) also referred to as “KDQNATK”.

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein. Thus, in an embodiment, the present invention provides a modified pneumolysin protein having an amino acid sequence comprising one or more consensus sequence(s) selected from: D/E-X-N-Z-S/T (SEQ ID NO. 28) and K-D/E-X-N-Z-S/T-K (SEQ ID NO. 30), wherein X and Z are independently any amino acid apart from proline, which have been recombinantly introduced into the pneumolysin amino acid sequence of SEQ ID NO. 1 or a pneumolysin amino acid sequence at least 80%, 85%, 90%, 92%, 95%, 96%, 97%, 98% or 99% identical to SEQ ID NO. 1. The present invention also provides a method for preparing a modified pneumolysin protein wherein one or more consensus sequence(s) selected from: D/E-X-N-Z-S/T (SEQ ID NO. 28) and K-D/E-X-N-Z-S/T-K (SEQ ID NO. 30), wherein X and Z are independently any amino acid apart from proline, are recombinantly introduced into the pneumolysin amino acid sequence of SEQ ID NO. 1 or a pneumolysin amino acid sequence at least 80%, 85%, 90%, 92%, 95%, 96%, 97%, 98% or 99% identical to SEQ ID NO. 1 (i.e. a recombinant modified pneumolysin protein). In certain embodiments, the classical 5 amino acid glycosylation consensus sequence (D/E-X-N-Z-S/T (SEQ ID NO. 28)) may be extended by lysine residues for more efficient glycosylation (e.g. K-D/E-X-N-Z-S/T-K (SEQ ID NO. 30)), and thus the inserted consensus sequence may encode 5, 6, or 7 amino acids that should be inserted or that replace acceptor protein amino acids.

In one embodiment, the modified pneumolysin protein of the invention comprises (or consists of) an amino acid sequence which is at least 80%, 85%, 90%, 92%, 95%, 96%, 97%, 98%, 99% or 100% identical to the sequence of SEQ ID NO. 2, said amino acid sequence comprising a D/E-X-N-Z-S/T (SEQ ID NO. 28) or K-D/E-X-N-Z-S/T-K (SEQ ID NO. 30) consensus sequence wherein X and Z are independently any amino acid apart from proline (e.g. K-D/E-X-N-Z-S/T-K (SEQ ID NO. 30) or K-D-Q-N-A-T-K (SEQ ID NO. 31)). In an embodiment, the modified pneumolysin protein of the invention comprises (or consists of) the amino acid sequence of SEQ ID NO. 2. In an embodiment, the modified pneumolysin protein of the invention comprises (or consists of) the amino acid sequence of SEQ ID NO. 2 with an N-terminal serine (i.e. a serine residue is added at the N-terminus).

Because pneumolysin is a toxin, it needs to be detoxified (i.e. rendered non-toxic to a mammal, e.g. human, when provided at a dosage suitable for protection) before it can be administered in vivo. A modified pneumolysin protein of the invention may be genetically detoxified (i.e. by mutation). The genetically detoxified sequences may remove undesirable activities such as membrane permeation, cell lysis, and cytolytic activity against human erythrocytes and other cells, in order to reduce the toxicity, whilst retaining the ability to induce anti-pneumolysin protective and/or neutralizing antibodies following administration to a human. For example, as described herein, a modified pneumolysin protein may be altered so that it is biologically inactive whilst still maintaining its immunogenic epitopes, see, for example, WO90/06951, Berry et al. (Infect Immun, 67:981-985 (1999)) and WO99/03884.

The modified pneumolysin proteins of the invention may be genetically detoxified by one or more point mutations. For example, a conserved cysteine-containing motif found near the C-terminus has been implicated in the lytic activity. Mutations of Ply have been suggested to lower this toxicity (WO90/06951, WO99/03884). Further detoxifying mutations of Ply are described in WO1999/003884, WO2005/076696, WO2005/108580. In one aspect, the modified pneumolysin proteins of the invention may be detoxified by amino acid substitutions as described in Oloo et al. (2011) (Oloo E. O., et al, J Biol Chem. 2011 Apr. 8; 286(14):12133), for example G₂₉₃ to C, T₆₅ to C and/or C₄₂₈ to A. For example, the modified pneumolysin proteins of the invention may comprise (i) at least one amino acid substitution selected from G₂₉₃ to C, T₆₅ to C and C₄₂₈ to A, (ii) at two amino acid substitutions selected from G₂₉₃ to C, T₆₅ to C and C₄₂₈ to A (e.g. G₂₉₃ to C and T₆₅ to C), or (iii) three amino acid substitutions G₂₉₃ to C, T₆₅ to C and C₄₂₈ to A (see also WO2010/071986). In an embodiment, the modified pneumolysin protein of the invention may be detoxified by introduction of amino acid substitutions T₆₅ to C and G₂₉₃ to C to form a disulfide cross-link. In another aspect, the modified pneumolysin protein of the invention may be detoxified by amino acid substitutions as described in Taylor et al. PLOS ONE 8(4): e61300 (2013), for example A₃₇₀ to E, W₄₃₃ to E and/or L₄₆₀ to E. For example, the modified pneumolysin proteins of the invention may comprise (iv) at least one amino acid substitution selected from A₃₇₀ to E, W₄₃₃ to E and L₄₆₀ to E, (v) at least two amino acid substitutions selected from A₃₇₀ to E, W₄₃₃ to E and L₄₆₀ to E or (vi) three amino acid substitutions A₃₇₀ to E, W₄₃₃ to E and L₄₆₀ to E. The modified pneumolysin protein of the invention may also be detoxified combination of the amino acid substitutions (i) to (vi) described above. The modified pneumolysin protein of the invention may comprise for example: (a) at least one amino acid substitution selected from G₂₉₃ to C, T₆₅ to C, C₄₂₈ to A, A₃₇₀ to E, W₄₃₃ to E and L₄₆₀ to E, (b) at least two amino acid substitutions selected from G₂₉₃ to C, T₆₅ to C, C₄₂₈ to A, A₃₇₀ to E, W₄₃₃ to E and L₄₆₀ to E, (c) at least three amino acid substitutions selected from G₂₉₃ to C, T₆₅ to C, C₄₂₈ to A, A₃₇₀ to E, W₄₃₃ to E and L₄₆₀ to E, (d) at least four amino acid substitutions selected from G₂₉₃ to C, T₆₅ to C, C₄₂₈ to A, A₃₇₀ to E, W₄₃₃ to E and L₄₆₀ to E, (e) at least five amino acid substitutions selected from G₂₉₃ to C, T₆₅ to C, C₄₂₈ to A, A₃₇₀ to E, W₄₃₃ to E and L₄₆₀ to E, or (f) six amino acid substitutions G₂₉₃ to C, T₆₅ to C, C₄₂₈ to A, A₃₇₀ to E, W₄₃₃ to E and L₄₆₀ to E. In an embodiment, the modified pneumolysin protein of the invention comprises five amino acid substitutions: G₂₉₃ to C, C₄₂₈ to A, A₃₇₀ to E, W₄₃₃ to E and L₄₆₀ to E. In an embodiment, the modified pneumolysin protein of the invention comprises six amino acid substitutions: G₂₉₃ to C, T₆₅ to C, C₄₂₈ to A, A₃₇₀ to E, W₄₃₃ to E and L₄₆₀ to E. Thus, in a modified pneumolysin protein of the invention, the amino acid sequence may be further modified by at least one amino acid substitution selected from G₂₉₃ to C, T₆₅ to C, C₄₂₈ to A, A₃₇₀ to E, W₄₃₃ to E and L₄₆₀ to E, with reference to the amino acid sequence of SEQ ID NO. 1 (or an equivalent position in an amino acid sequence at least 80%, 85%, 90%, 92%, 95%, 96%, 97%, 98% or 99% identical to SEQ ID NO. 1).

The amino acid numbers referred to herein correspond to the amino acids in SEQ ID NO. 1 and as described above, a person skilled in the art can determine equivalent amino acid positions in an amino acid sequence at least 80%, 85%, 90%, 92%, 95%, 96%, 97%, 98% or 99% identical to SEQ ID NO. 1 by alignment. For example, the skilled person would understand that L₄₆₀ in SEQ ID NO. 1 corresponds to L₄₆₅ in SEQ ID NOs. 2-7 and reference to L₄₆₀ to E includes L₄₆₅E in SEQ ID NOs. 8, 10 and 12 and L₄₆₆E in SEQ ID NO. 9 and SEQ ID NO. 11.

In one aspect, the modified pneumolysin protein of the invention comprises (or consists of) an amino acid sequence which is at least 80%, 85%, 90%, 92%, 95%, 97%, 98%, 99% or 100% identical to the sequence of SEQ ID NO: 3-10, said amino acid sequence comprising a D/E-X-N-Z-S/T (SEQ ID NO. 28) or K-D/E-X-N-Z-S/T-K (SEQ ID NO. 30) consensus sequence wherein X and Z are independently any amino acid apart from proline (e.g. K-D/E-X-N-Z-S/T-K (SEQ ID NO. 30) or K-D-Q-N-A-T-K (SEQ ID NO. 31)) and at least one amino acid substitution selected from G₂₉₃ to C, T₆₅ to C, C₄₂₈ to A, A₃₇₀ to E, W₄₃₃ to E and L₄₆₀ to E. In an embodiment, the modified pneumolysin protein of the invention comprises (or consists of) the amino acid sequence of SEQ ID NO: 3-10. In another embodiment, the modified pneumolysin protein of the invention comprises (or consists of) the amino acid sequence of SEQ ID NO: 3-8 with an N-terminal serine (i.e. a serine residue is added at the N-terminus). In an embodiment, the modified pneumolysin of the invention comprises (or consists of) an amino acid sequence which is at least 80%, 85%, 90%, 92%, 95%, 97%, 98%, 99% or 100% identical to the sequence of SEQ ID NO. 3. In an embodiment, the modified pneumolysin of the invention comprises (or consists of) an amino acid sequence which is at least 80%, 85%, 90%, 92%, 95%, 97%, 98%, 99% or 100% identical to the sequence of SEQ ID NO. 4. In an embodiment, the modified pneumolysin of the invention comprises (or consists of) an amino acid sequence which is at least 80%, 85%, 90%, 92%, 95%, 97%, 98%, 99% or 100% identical to the sequence of SEQ ID NO. 4. In an embodiment, the modified pneumolysin of the invention comprises (or consists of) an amino acid sequence which is at least 80%, 85%, 90%, 92%, 95%, 97%, 98%, 99% or 100% identical to the sequence of SEQ ID NO. 5. In an embodiment, the modified pneumolysin of the invention comprises (or consists of) an amino acid sequence which is at least 80%, 85%, 90%, 92%, 95%, 97%, 98%, 99% or 100% identical to the sequence of SEQ ID NO. 6. In an embodiment, the modified pneumolysin of the invention comprises (or consists of) an amino acid sequence which is at least 80%, 85%, 90%, 92%, 95%, 97%, 98%, 99% or 100% identical to the sequence of SEQ ID NO. 7. In an embodiment, the modified pneumolysin of the invention comprises (or consists of) an amino acid sequence which is at least 80%, 85%, 90%, 92%, 95%, 97%, 98%, 99% or 100% identical to the sequence of SEQ ID NO. 8. In an embodiment, the modified pneumolysin of the invention comprises (or consists of) an amino acid sequence which is at least 80%, 85%, 90%, 92%, 95%, 97%, 98%, 99% or 100% identical to the sequence of SEQ ID NO. 9. In an embodiment, the modified pneumolysin of the invention comprises (or consists of) an amino acid sequence which is at least 80%, 85%, 90%, 92%, 95%, 97%, 98%, 99% or 100% identical to the sequence of SEQ ID NO. 10. In an embodiment, the present invention provides a modified pneumolysin protein having an amino acid sequence at least 80%, 85%, 90%, 95%, 96%, 97%, 98%, 99% or 100% identical to SEQ ID NO. 9 or SEQ ID NO. 10, said amino acid sequence comprising a D/E-X-N-Z-S/T (SEQ ID NO. 28) consensus sequence wherein X and Z are independently any amino acid apart from proline (e.g. K-D/E-X-N-Z-S/T-K (SEQ ID NO. 30) or K-D-Q-N-A-T-K (SEQ ID NO. 31)) and at least one amino acid substitution selected from G₂₉₃ to C, T₆₅ to C, C₄₂₈ to A, A₃₇₀ to E, W₄₃₃ to E and L₄₆₀ to E. In an embodiment, the present invention provides a modified pneumolysin protein having an amino acid sequence at least 80%, 85%, 90%, 95%, 96%, 97%, 98%, 99% or 100% identical to SEQ ID NO. 11 or SEQ ID NO. 12, said amino acid sequence comprising a D/E-X-N-Z-S/T (SEQ ID NO. 28) consensus sequence wherein X and Z are independently any amino acid apart from proline (e.g. K-D/E-X-N-Z-S/T-K (SEQ ID NO. 30) or K-D-Q-N-A-T-K (SEQ ID NO. 31)) and at least one amino acid substitution selected from G₂₉₃ to C, T₆₅ to C, C₄₂₈ to A, A₃₇₀ to E, W₄₃₃ to E and L₄₆₀ to E. In an embodiment, the present invention provides a modified pneumolysin protein having an amino acid sequence of SEQ ID NO. 11 or SEQ ID NO. 12.

In an embodiment, the present invention provides a modified pneumolysin protein having an amino acid sequence at least 80%, 85%, 90%, 95%, 96%, 97%, 98%, 99% or 100% identical to SEQ ID NO. 9 or SEQ ID NO. 10 and comprising an amino acid sequence selected from SEQ ID NOs. 23 to 27 wherein X1, X2 and X3 are any amino acid, suitably wherein X1 is C (cysteine), X2 is E (glutamic acid) and X3 is A (alanine).

The activity of the modified pneumolysin protein of the invention may be assayed and characterized by methods described for example in Nato, et al. Infect Immun. 59(12):4641-4646 (1991), Taylor et al. PLOS ONE 8(4): e61300 (2013)). An in vitro hemolysis assay may be used to measure the hemolytic (e.g. cytolytic) activity of modified pneumolysin protein relative to wild-type pneumolysin. A hemolysis inhibition assay may be used to measure the ability of antisera raised against a modified pneumolysin protein of the invention to inhibit hemolysis by pneumolysin, and (typically) comparing anti-(modified pneumolysin) antisera to anti-(wild-type pneumolysin) antisera. For example, a suitable modified pneumolysin protein of the invention may be one that exhibits lower hemolytic activity than wild-type pneumolysin (e.g. via an in vitro hemolysis assay). For instance, a suitable modified pneumolysin protein may have a specific activity (as determined using the in vitro hemolysis assay) of about (referring to each of the following values independently) 0%, 0.0005%, 0.001%, 0.005%, 0.01%, 0.05%, 0.1%, 0.5%, 1%, 5% or <10% the specific activity of the wild-type pneumolysin. A suitable modified pneumolysin protein of the invention may also be one that, following administration to a host, causes the host to produce antibodies that inhibit hemolysis by wild-type pneumolysin (e.g. via a hemolysis inhibition assay), is immunogenic (e.g. induces the production of antibodies against wtPLY), and/or protective (e.g. induces an immune response that protects the host against infection by or limits an already-existing infection). Assays may be used as described in the Examples.

In an embodiment, the modified pneumolysin protein of the invention further comprises a “peptide tag” or “tag”, i.e. a sequence of amino acids that allows for the isolation and/or identification of the modified pneumolysin protein. For example, adding a tag to a modified pneumolysin protein of the invention can be useful in the purification of that protein and, hence, the purification of conjugate vaccines comprising the tagged modified pneumolysin protein. Exemplary tags that can be used herein include, without limitation, histidine (HIS) tags (e.g. hexa histidine-tag, or 6×His-Tag), FLAG-TAG, and HA tags. In one embodiment, the tag is a hexa-histidine tag. In certain embodiments, the tags used herein are removable, e.g. removal by chemical agents or by enzymatic means, once they are no longer needed, e.g. after the protein has been purified. Optionally the peptide tag is located at the C-terminus of the amino acid sequence. Optionally the peptide tag comprises six histidine residues at the C-terminus of the amino acid sequence. In one aspect, the modified pneumolysin protein of the invention comprises (or consists of) an amino acid sequence which is at least 80%, 85%, 90%, 92%, 95%, 97%, 98%, 99% or 100% identical to the sequence of SEQ ID NO. 11 or SEQ ID NO. 12, said amino acid sequence comprising a D/E-X-N-Z-S/T (SEQ ID NO. 28) consensus sequence wherein X and Z are independently any amino acid apart from proline (e.g. K-D/E-X-N-Z-S/T-K (SEQ ID NO. 30) or K-D-Q-N-A-T-K (SEQ ID NO. 31)) and at least one amino acid substitution selected from G₂₉₃ to C, T₆₅ to C, C₄₂₈ to A, A₃₇₀ to E, W₄₃₃ to E and L₄₆₀ to E and a peptide tag (e.g. six histidine residues at the C-terminus of the amino acid sequence). Optionally, the modified pneumolysin protein of the invention, has an amino acid sequence at least 97%, 98%, 99% or 100% identical to SEQ ID NO. 11 or SEQ ID NO. 12.

In an embodiment, the modified pneumolysin protein of the invention comprises a signal sequence which is capable of directing the pneumolysin protein to the periplasm of a host cell (e.g. bacterium). In a specific embodiment, the signal sequence is from E. coli DsbA [MKKIWLALAGLVLAFSASA (Seq ID NO. 13)], E. coli outer membrane porin A (OmpA) [MKKTAIAIAVALAGFATVAQA (Seq ID NO. 14)], E. coli maltose binding protein (MalE) [MKIKTGARILALSALTTMMFSASALA (Seq ID NO. 15)], Erwinia carotovorans pectate lyase (PelB) [MKYLLPTAAAGLLLLAAQPAMA (Seq ID NO. 16)], heat labile E. coli enterotoxin LTIIb [MSFKKIIKAFVIMAALVSVQAHA (Seq ID NO. 17)], Bacillus endoxylanase XynA [MFKFKKKFLVGLTAAFMSISMFSATASA (Seq ID NO. 18)], E. coli flagellin (FlgI) [MIKFLSALILLLVTTAAQA (Seq ID NO. 19)], TolB [MKQALRVAFGFLILWASVLHA (Seq ID NO. 20)] or SipA [MKMNKKVLLTSTMAASLLSVASVQAS (SEQ ID NO.70)]. In an embodiment, the signal sequence has an amino acid sequence at least 80%, 85%, 90%, 92%, 95%, 96%, 97%, 98%, 99% or 100% identical to a SEQ ID NO. 13-20 or 70 (signal seq). In one aspect, the signal sequence has an amino acid sequence at least 80%, 85%, 90%, 95%, 96%, 97%, 98%, 99% or 100% identical to TolB [MKQALRVAFGFLILWASVLHA (Seq ID NO. 20)]. For example, a signal sequence having an amino acid sequence at least 80%, 85%, 90%, 95%, 96%, 97%, 98%, 99% or 100% identical to TolB [MKQALRVAFGFLILWASVLHA (Seq ID NO. 20)] may be used for CP12F and CP4. In another aspect, the signal sequence has an amino acid sequence at least 80%, 85%, 90%, 95%, 96%, 97%, 98%, 99% or 100% identical to (PelB) [MKYLLPTAAAGLLLLAAQPAMA (Seq ID NO. 16)]. For example, a signal sequence having an amino acid sequence at least 80%, 85%, 90%, 95%, 96%, 97%, 98%, 99% or 100% identical to (PelB) [MKYLLPTAAAGLLLLAAQPAMA (Seq ID NO. 16)] may be used for CP33F. another aspect, the signal sequence has an amino acid sequence at least 80%, 85%, 90%, 95%, 96%, 97%, 98%, 99% or 100% identical to SipA [MKMNKKVLLTSTMAASLLSVASVQAS (SEQ ID NO.70)]. In an embodiment, a modified pneumolysin protein of the invention further comprises a signal sequence SEQ ID NO. 70 (SipA), SEQ ID NO. 16 (PelB) or SEQ ID NO. 20 (TolB), suitably at the N-terminus. In an embodiment, a modified pneumolysin protein of the invention further comprises a signal sequence SEQ ID NO. 16 (PelB) or SEQ ID NO. 20 (TolB), suitably at the N-terminus. In an embodiment, a modified pneumolysin protein of the invention has an amino acid sequence at least 80%, 85%, 90%, 95%, 96%, 97%, 98%, 99% or 100% identical to an amino acid sequence selected from SEQ ID NO. 32 or SEQ ID NO. 51 modified in that the amino acid sequence comprises one or more consensus sequence(s) selected from: D/E-X-N-Z-S/T (SEQ ID NO. 28) and K-D/E-X-N-Z-S/T-K (SEQ ID NO. 30), wherein X and Z are independently any amino acid apart from proline.

In an embodiment, an alanine residue is added between the signal sequence and the start of the sequence of the mature protein. Such an alanine residue has the advantage of leading to more efficient cleavage of the leader sequence.

In one aspect, the modified pneumolysin protein of the invention comprises (or consists of) an amino acid sequence which is at least 97%, 98%, 99% or 100% identical to the sequence of SEQ ID NO. 21 or SEQ ID NO. 22, said amino acid sequence comprising a D/E-X-N-Z-S/T (SEQ ID NO. 28) consensus sequence wherein X and Z are independently any amino acid apart from proline (e.g. K-D/E-X-N-Z-S/T-K (SEQ ID NO. 30) or K-D-Q-N-A-T-K (SEQ ID NO. 31)) and at least one amino acid substitution selected from G₂₉₃ to C, T₆₅ to C, C₄₂₈ to A, A₃₇₀ to E, W₄₃₃ to E and L₄₆₀ to E (and optionally comprising six histidine residues at the C-terminus of the amino acid sequence and a signal sequence). In an embodiment, a modified pneumolysin protein of the invention has an amino acid sequence at least 97%, 98%, 99% or 100% identical to an amino acid sequence selected from SEQ ID NO. 21 or SEQ ID NO. 22. In another embodiment, the present invention provides a modified pneumolysin protein having an amino acid sequence of SEQ ID NO. 21 or SEQ ID NO. 22. In another aspect, the modified pneumolysin protein of the invention comprises (or consists of) an amino acid sequence which is at least 97%, 98%, 99% or 100% identical to the sequence selected from SEQ ID NOs. 33-50 or 52-69, said amino acid sequence comprising a D/E-X-N-Z-S/T (SEQ ID NO. 28) consensus sequence wherein X and Z are independently any amino acid apart from proline (e.g. K-D/E-X-N-Z-S/T-K (SEQ ID NO. 30) or K-D-Q-N-A-T-K (SEQ ID NO. 31)) and at least one amino acid substitution selected from G₂₉₃ to C, T₆₅ to C, C₄₂₈ to A, A₃₇₀ to E, W₄₃₃ to E and L₄₆₀ to E (and optionally comprising six histidine residues at the C-terminus of the amino acid sequence and a signal sequence). In an embodiment, a modified pneumolysin protein of the invention has an amino acid sequence at least 97%, 98%, 99% or 100% identical to an amino acid sequence selected from SEQ ID NOs. 33-50 or SEQ ID NOs. 52-69. In another embodiment, the present invention provides a modified pneumolysin protein having an amino acid sequence selected from SEQ ID NOs. 33-50 or SEQ ID NOs. 52-69.

A further aspect of the invention is a polynucleotide encoding a modified pneumolysin protein of the invention. For example, a polynucleotide encoding a modified pneumolysin protein, having a nucleotide sequence that encodes a polypeptide with an amino acid sequence that is at least 97%, 98%, 99% or 100% identical to any one of SEQ ID NO. 2-12, 21 or 22. For example, a polynucleotide encoding a modified pneumolysin protein, having a nucleotide sequence that encodes a polypeptide with an amino acid sequence that is at least 97%, 98%, 99% or 100% identical to any one of SEQ ID NOs. 33-50 or 52-69. A vector comprising such a polynucleotide is a further aspect of the invention.

### Conjugates

The present invention also provides a conjugate (e.g. bioconjugate) comprising (or consisting of) a modified pneumolysin protein of the invention, wherein the modified pneumolysin protein is linked to an antigen, e.g. covalently linked to an antigen.

In an embodiment, the conjugate comprises a conjugate (e.g. bioconjugate) comprising (or consisting of) a modified pneumolysin protein of the invention having an amino acid sequence which is at least 97%, 98%, 99% or 100% identical to the sequence of SEQ ID NO. 2-12 covalently linked to an antigen, wherein the antigen is linked (either directly or through a linker) to an amino acid residue of the modified pneumolysin protein.

In an embodiment, the modified pneumolysin protein is covalently linked to the antigen through a chemical linkage obtainable using a chemical conjugation method (i.e. the conjugate is produced by chemical conjugation).

In an embodiment, the chemical conjugation method is selected from the group consisting of carbodiimide chemistry, reductive animation, cyanylation chemistry (for example CDAP chemistry), maleimide chemistry, hydrazide chemistry, ester chemistry, and N-hydroysuccinimide chemistry. Conjugates can be prepared by direct reductive amination methods as described in, US200710184072 (Hausdorff) U.S. Pat. No. 4,365,170 (Jennings) and U.S. Pat. No. 4,673,574 (Anderson). Other methods are described in EP-0-161-188, EP-208375 and EP-0-477508. The conjugation method may alternatively rely on activation of the saccharide with 1-cyano-4-dimethylamino pyridinium tetrafluoroborate (CDAP) to form a cyanate ester. Such conjugates are described in PCT published application WO 93/15760 Uniformed Services University and WO 95/08348 and WO 96/29094. See also Chu C. et al Infect. Immunity, 1983 245 256.

In general the following types of chemical groups on a modified pneumolysin protein can be used for coupling/conjugation:

A) Carboxyl (for instance via aspartic acid or glutamic acid). In one embodiment this group is linked to amino groups on saccharides directly or to an amino group on a linker with carbodiimide chemistry e.g. with EDAC.

B) Amino group (for instance via lysine). In one embodiment this group is linked to carboxyl groups on saccharides directly or to a carboxyl group on a linker with carbodiimide chemistry e.g. with EDAC. In another embodiment this group is linked to hydroxyl groups activated with CDAP or CNBr on saccharides directly or to such groups on a linker; to saccharides or linkers having an aldehyde group; to saccharides or linkers having a succinimide ester group.

C) Sulphydryl (for instance via cysteine). In one embodiment this group is linked to a bromo or chloro acetylated saccharide or linker with maleimide chemistry. In one embodiment this group is activated/modified with bis diazobenzidine.

D) Hydroxyl group (for instance via tyrosine). In one embodiment this group is activated/modified with bis diazobenzidine.

E) Imidazolyl group (for instance via histidine). In one embodiment this group is activated/modified with bis diazobenzidine.

F) Guanidyl group (for instance via arginine).

G) Indolyl group (for instance via tryptophan).

On a saccharide, in general the following groups can be used for a coupling: OH, COOH or NH₂. Aldehyde groups can be generated after different treatments such as: periodate, acid hydrolysis, hydrogen peroxide, etc.

### Direct Coupling Approaches:

Saccharide-OH+CNBr or CDAP→cyanate ester+NH₂-Protein→conjugate
Saccharide-aldehyde+NH₂-Protein→Schiff base+NaCNBH3→conjugate

### Saccharide-COOH+NH₂-Protein+EDAC→conjugate

### Saccharide-NH₂+COOH-Protein+EDAC→conjugate

### Indirect Coupling Via Spacer (Linker) Approaches:

Saccharide-OH+CNBr or CDAP→cyanate ester+NH₂—NH₂→saccharide-NH₂+COOH-Protein+EDAC→conjugate
Saccharide-OH+CNBr or CDAP→cyanate ester+NH₂—SH→saccharide-SH+SH-Protein (native Protein with an exposed cysteine or obtained after modification of amino groups of the protein by SPDP for instance)→saccharide-S—S-Protein
Saccharide-OH+CNBr or CDAP→cyanate ester+NH₂—SH→saccharide-SH+maleimide-Protein (modification of amino groups)→conjugate
Saccharide-OH+CNBr or CDAP→cyanate ester+NH₂—SH→Saccharide-SH+haloacetylated-Protein→Conjugate
Saccharide-COOH+EDAC+NH₂—NH₂→saccharide-NH₂+EDAC+COOH-Protein→conjugate
Saccharide-COOH+EDAC+NH₂—SH→saccharide-SH+SH-Protein (native Protein with an exposed cysteine or obtained after modification of amino groups of the protein by SPDP for instance)→saccharide-S—S-Protein
Saccharide-COOH+EDAC+NH₂—SH→saccharide-SH+maleimide-Protein (modification of amino groups)→conjugate

### Saccharide-COOH+EDAC+NH₂—SH→Saccharide-SH+haloacetylated-Protein→Conjugate

Saccharide-Aldehyde+NH₂—NH₂→saccharide-NH2+EDAC+COOH-Protein→conjugate

Note: instead of EDAC above, any suitable carbodiimide may be used.

In an embodiment, the antigen is directly linked to the modified pneumolysin protein.

In an embodiment, the antigen is attached to the modified pneumolysin protein via a linker. Optionally, the linker is selected from the group consisting of linkers with 4-12 carbon atoms, bifunctional linkers, linkers containing 1 or 2 reactive amino groups at the end, B-proprionamido, nitrophenyl-ethylamine, haloacyl halides, 6-aminocaproic acid and ADH. The activated saccharide may thus be coupled directly or via a spacer (linker) group to an amino group on the modified pneumolysin protein. For example, the spacer could be cystamine or cysteamine to give a thiolated polysaccharide which could be coupled to the modified pneumolysin via a thioether linkage obtained after reaction with a maleimide-activated modified pneumolysin protein (for example using GMBS (4-Maleimidobutyric acid N-hydroxysuccinimide ester)) or a haloacetylated modified pneumolysin protein (for example using SIAB (succinimidyl (4-iodoacetyl)aminobenzoate), or SIA (succinimidyl iodoacetate), or SBAP (succinimidyl-3-(bromoacetamide)propionate)). In an embodiment, the cyanate ester (optionally made by CDAP chemistry) is coupled with hexane diamine or ADH (adipic acid dihydrazide) and the amino-derivatised saccharide is conjugated to the modified pneumolysin protein using carbodiimide (e.g. 1-Ethyl-3-(3-dimethylaminopropyl)carbodiimide (EDAC or EDC)) chemistry via a carboxyl group on the protein modified pneumolysin. Such conjugates are described in PCT published application WO 93/15760 Uniformed Services University and WO 95/08348 and WO 96/29094.

In an embodiment, the amino acid residue on the modified pneumolysin protein to which the antigen is linked is not an asparagine residue and in this case, the conjugate is typically produced by chemical conjugation. In an embodiment, the amino acid residue on the modified pneumolysin protein to which the antigen is linked is selected from the group consisting of: Ala, Arg, Asp, Cys, Gly, Glu, Gin, His, lie, Leu, Lys, Met, Phe, Pro, Ser, Thr, Trp, Tyr, and Val. Optionally, the amino acid is: an amino acid containing a terminal amine group, a lysine, an arginine, a glutaminic acid, an aspartic acid, a cysteine, a tyrosine, a histidine or a tryptophan. Optionally, the antigen is covalently linked to amino acid on the modified pneumolysin protein selected from: aspartic acid, glutamic acid, lysine, cysteine, tyrosine, histidine, arginine or tryptophan.

In an embodiment, the amino acid residue on the modified pneumolysin protein to which the antigen is linked is not part of the D/E-X-N-Z-S/T (SEQ ID NO. 28) and K-D/E-X-N-Z-S/T-K (SEQ ID NO. 30) consensus sequence. In an embodiment, the amino acid residue on the modified pneumolysin protein to which the antigen is linked is not the asparagine residue in the D/E-X-N-Z-S/T (SEQ ID NO. 28) and K-D/E-X-N-Z-S/T-K (SEQ ID NO. 30) consensus sequence.

Alternatively, in another embodiment, the antigen is linked to an amino acid on the modified pneumolysin protein selected from asparagine, aspartic acid, glutamic acid, lysine, cysteine, tyrosine, histidine, arginine or tryptophan (e.g. asparagine). In another embodiment, the amino acid residue on the modified pneumolysin protein to which the antigen is linked is an asparagine residue. In another embodiment, the amino acid residue on the modified pneumolysin protein to which the antigen is linked is part of the D/E-X-N-Z-S/T (SEQ ID NO. 28) and K-D/E-X-N-Z-S/T-K (SEQ ID NO. 30) consensus sequence (e.g. the asparagine in the D/E-X-N-Z-S/T (SEQ ID NO. 28) and K-D/E-X-N-Z-S/T-K (SEQ ID NO. 30) consensus sequence).

### Antigens

In an embodiment, the antigen in a conjugate (e.g. bioconjugate) of the invention is a saccharide such as a bacterial capsular saccharide, a bacterial lipopolysaccharide or a bacterial oligosaccharide. In an embodiment the antigen is a bacterial capsular saccharide.

The saccharides may be selected from a group consisting of: N. meningitidis serogroup A capsular saccharide (MenA), N. meningitidis serogroup C capsular saccharide (MenC), N. meningitidis serogroup Y capsular saccharide (MenY), N. meningitidis serogroup W capsular saccharide (MenW), H. influenzae type b capsular saccharide (Hib), Group B Streptococcus group I capsular saccharide, Group B Streptococcus group II capsular saccharide, Group B Streptococcus group III capsular saccharide, Group B Streptococcus group IV capsular saccharide, Group B Streptococcus group V capsular saccharide, Staphylococcus aureus type 5 capsular saccharide, Staphylococcus aureus type 8 capsular saccharide, Vi saccharide from Salmonella typhi, N. meningitidis LPS (such as L3 and/or L2), M. catarrhalis LPS, H. influenzae LPS, Shigella O-antigens, P. aeruginosa O-antigens, E. coli O-antigens or S. pneumoniae capsular polysaccharide.

In an embodiment, the antigen is a bacterial capsular saccharide from S. pneumoniae. The bacterial capsular saccharide from Streptococcus pneumoniae may be selected from a Streptococcus pneumoniae serotype 1, 2, 3, 4, 5, 6A, 6B, 7A, 7B, 7C, 8, 9A, 9L, 9N, 9V, 10A, 10B, 10C, 10F, 11A, 11B, 11C, 11D, 11F, 12A, 12B, 12F, 13, 14, 15A, 15B, 15C, 15F, 16A, 16F, 17A, 17F, 18A, 18B, 1, 18F, 19A, 19B, 19C, 19F, 20, 21, 22A, 22F, 23A, 23B, 23F, 24A, 24B, 24F, 25A, 25F, 26, 27, 28A, 28F, 29, 31, 32A, 32F, 33A, 33B, 33C, 33D, 33F, 34, 35A, 35B, 35C, 35D, 35F, 36, 37, 38, 39, 40, 41A, 41F, 42, 43, 44, 45, 46, 47A, 47F or 48 capsular saccharide. For example, the antigen may be an S. pneumoniae capsular saccharide from serotype: 1, 2, 3, 4, 5, 6A, 6B, 7F, 8, 9N, 9V, 10A, 11A, 12F, 14, 15B, 17F, 18C, 19A, 19F, 20, 22F, 23F or 33F. In one particular aspect, the antigen is a bacterial capsular saccharide from Streptococcus pneumoniae serotype 4. In another particular aspect, the antigen is a bacterial capsular saccharide from Streptococcus pneumoniae serotype 12F. In another particular aspect, the antigen is a bacterial capsular saccharide from Streptococcus pneumoniae serotype 33F.

In an embodiment of the invention, the antigen is a repeat unit of a bacterial capsular saccharide from S. pneumoniae. In an embodiment of the invention, the antigen comprises a repeat unit of a bacterial capsular saccharide from S. pneumoniae serotype 4. For example, CP4 has a repeat unit structure containing a GalNAc at the reducing end. The complete structure is: -1,4-(2,3 S pyr)-a-D-Gal-1,3-a-D-ManNAc-1,3-a-L-FucNAc-1,3-a-D-GalNAc. In an embodiment of the invention, the antigen comprises a repeat unit of a bacterial capsular saccharide from S. pneumoniae serotype 12F. In an embodiment of the invention, the antigen comprises a repeat unit of a bacterial capsular saccharide from S. pneumoniae serotype 33F.

In a further embodiment of the invention, the antigen is a hybrid oligosaccharide or polysaccharide having a structure:

(B)ₙ-A→

wherein A is an oligosaccharide repeat unit containing at least 2, 3, 4, 5, 6, 7 or 8 monosaccharides, with a hexose monosaccharide derivative at the reducing end (indicated by arrow);
wherein B is an oligosaccharide repeat unit containing at least 2, 3, 4, 5, 6, 7 or 8 monosaccharides;
wherein A and B are different oligosaccharide repeat units; and
wherein n is either at least 2, 3, 4, 5, 6, 7, 8, 9, 10, 11, 12, 13, 14, 15, 16, 17, 18, 19, 20, 30, 40, 50, 60, 70, 80, 100 or at least 200: or
wherein n is at least 1, 2, 3, 4, 5, 6, 7, 8, 9, 10, 11, 12, 13, 14, 15, 16, 17, 18, 19, 20, 30, 40, 50, 60, 70, 80, 100 or at least 200.

In an embodiment, A is an oligosaccharide containing no more than 20, 15, 12, 10, 9, or 8 monosaccharides. In an embodiment, B is an oligosaccharide containing no more than 20, 15, 12, 10, 9, or 8 monosaccharides. In an embodiment, A and B are oligosaccharides containing no more than 20, 15, 12, 10, 9, or 8 monosaccharides. In an embodiment n is no more than 500, 400, 300, 200, 100, 90, 80, 70, 60, 50, 40, 30, 20, 10 or 5.

In an embodiment, A is an oligosaccharide repeat unit containing at least 3 monosaccharides and B is an oligosaccharide repeat unit containing at least 3 monosaccharides. In an embodiment, A is an oligosaccharide repeat unit containing at least 3 monosaccharides and B is an oligosaccharide repeat unit containing at least 3 monosaccharides and n is at least 5. In an embodiment, A is an oligosaccharide repeat unit containing at least 3 monosaccharides and B is an oligosaccharide repeat unit containing at least 3 monosaccharides and n is at least 20.

In an embodiment, A is an oligosaccharide repeat unit containing at least 4 monosaccharides and B is an oligosaccharide repeat unit containing at least 4 monosaccharides. In an embodiment, A is an oligosaccharide repeat unit containing at least 4 monosaccharides and B is an oligosaccharide repeat unit containing at least 4 monosaccharides and n is at least 5. In an embodiment, A is an oligosaccharide repeat unit containing at least 4 monosaccharides and B is an oligosaccharide repeat unit containing at least 4 monosaccharides and n is at least 20.

In an embodiment, A is an oligosaccharide repeat unit containing at least 5 monosaccharides and B is an oligosaccharide repeat unit containing at least 5 monosaccharides. In an embodiment, A is an oligosaccharide repeat unit containing at least 5 monosaccharides and B is an oligosaccharide repeat unit containing at least 5 monosaccharides and n is at least 5. In an embodiment, A is an oligosaccharide repeat unit containing at least 5 monosaccharides and B is an oligosaccharide repeat unit containing at least 5 monosaccharides and n is at least 20.

In an embodiment, A is an oligosaccharide repeat unit containing at least 6 monosaccharides and B is an oligosaccharide repeat unit containing at least 6 monosaccharides. In an embodiment, A is an oligosaccharide repeat unit containing at least 6 monosaccharides and B is an oligosaccharide repeat unit containing at least 6 monosaccharides and n is at least 5. In an embodiment, A is an oligosaccharide repeat unit containing at least 6 monosaccharides and B is an oligosaccharide repeat unit containing at least 6 monosaccharides and n is at least 20.

In an embodiment, A is an oligosaccharide repeat unit containing 2-8 monosaccharides and B is an oligosaccharide repeat unit containing 2-8 monosaccharides. In an embodiment, A is an oligosaccharide repeat unit containing 2-8 monosaccharides and B is an oligosaccharide repeat unit containing 2-8 monosaccharides and n is at least 5 and no more than 500. In an embodiment, A is an oligosaccharide repeat unit containing 2-8 monosaccharides and B is an oligosaccharide repeat unit containing 2-8 monosaccharides and n is at least 20 and no more than 100.

In an embodiment, A is an oligosaccharide repeat unit containing 2-10 monosaccharides and B is an oligosaccharide repeat unit containing 2-10 monosaccharides. In an embodiment, A is an oligosaccharide repeat unit containing 2-10 monosaccharides and B is an oligosaccharide repeat unit containing 2-10 monosaccharides and n is at least 5 and no more than 500. In an embodiment, A is an oligosaccharide repeat unit containing 2-10 monosaccharides and B is an oligosaccharide repeat unit containing 2-10 monosaccharides and n is at least 20 and no more than 100.

In an embodiment of the hybrid oligosaccharide or polysaccharide, the B oligosaccharide repeat contains a hexose monosaccharide at the reducing end of the repeat. In an embodiment of the hybrid oligosaccharide or polysaccharide, the hexose monosaccharide at the reducing end of the repeat is selected from the group consisting of glucose, galactose, rhamnose, arabinotol, fucose and mannose; suitably the group consists of glucose and galactose.

In an embodiment of the hybrid oligosaccharide or polysaccharide, the oligosaccharide repeat unit of A and the oligosaccharide repeat unit of B differ only by containing a different monosaccharide at the reducing end of the repeat.

In an embodiment of the hybrid oligosaccharide or polysaccharide, the oligosaccharide repeat unit of A is the repeat unit of the capsular saccharide of a bacterial capsular saccharide from S. pneumoniae as described above; for example the repeat unit of the capsular saccharide of a Streptococcus pneumoniae serotype 1, 2, 3, 4, 5, 6A, 6B, 7A, 7B, 7C, 8, 9A, 9L, 9N, 9V, 10A, 10B, 10C, 10F, 11A, 11B, 11C, 11D, 11F, 12A, 12B, 12F, 13, 14, 15A, 15B, 15C, 15F, 16A, 16F, 17A, 17F, 18A, 18B, 18C, 18F, 19A, 19B, 19C, 19F, 20, 21, 22A, 22F, 23A, 23B, 23F, 24A, 24B, 24F, 25A, 25F, 26, 27, 28A, 28F, 29, 31, 32A, 32F, 33A, 33B, 33C, 33D, 33F, 34, 35A, 35B, 35C, 35D, 35F, 36, 37, 38, 39, 40, 41A, 41F, 42, 43, 44, 45, 46, 47A, 47F or 48; suitably of serotype 33F. An example is CP33F, for which the repeat unit is composed of β-D-Galf-1,3-β-D-Gal-1,3-α-D-Gal(α1,2-D-Gal)-1,3-β-D-Galf-1,3-D-Glc-.

An aspect of the invention is a conjugate (e.g. bioconjugate) comprising a modified pneumolysin protein N-linked to an antigen, e.g. saccharide. In a conjugate (e.g. bioconjugate) comprising a modified pneumolysin protein containing a Asn-X-Ser/Thr consensus sequence (e.g. within D/E-X-N-Z-S/T (SEQ ID NO. 28) or K-D/E-X-N-Z-S/T-K (SEQ ID NO. 30)), the asparagine residue may be linked to the antigen, e.g. saccharide. A further aspect of the invention is a conjugate (e.g. bioconjugate) comprising a modified pneumolysin protein N-linked to a hybrid oligosaccharide or polysaccharide, wherein said hybrid oligosaccharide or polysaccharide is identical to a donor oligosaccharide or polysaccharide, with the exception of the fact that the hybrid oligosaccharide or polysaccharide comprises a hexose monosaccharide derivative at the reducing end of the first repeat unit in addition to comprising all of the monosaccharides of the donor oligosaccharide or polysaccharide. In other words, a conjugate (e.g. bioconjugate) comprising a modified pneumolysin protein containing a Asn-X-Ser/Thr consensus sequence (e.g. within D/E-X-N-Z-S/T (SEQ ID NO. 28) or K-D/E-X-N-Z-S/T-K (SEQ ID NO. 30)), the asparagine residue of which is linked to a hybrid oligosaccharide or polysaccharide, wherein said hybrid oligosaccharide or polysaccharide contains at least 2, 3, 4, 5, 6, 7, 8, 9, 10, 20, 30, 40 or 50 saccharide repeat units of a donor oligosaccharide or polysaccharide and a further repeat unit N-linked to the modified pneumolysin protein in which a hexose monosaccharide derivative is at the reducing end of said further repeat unit.

In an embodiment, the hexose monosaccharide derivative is any monosaccharide in which C-2 position is modified with an acetamido group. In one aspect, the hexose monosaccharide is selected from the group consisting of glucose, galactose, rhamnose, arabinotol, fucose and mannose (e.g. galactose). Suitable hexose monosaccharide derivatives include N-acetylglucosamine (GlcNAc), N-acetylgalactoseamine (GalNAc), HexNAc, deoxy HexNAc, 2,4-Diacetamido-2,4,6-trideoxyhexose (DATDH), N-acetylfucoseamine (FucNAc), or N-acetylquinovosamine (QuiNAc). A suitable hexose monosaccharide derivative is N-acetylglucosamine (GlcNAc).

In an embodiment, the hybrid oligosaccharide or polysaccharide is identical to a Gram positive bacterial capsular saccharide, with the exception of the fact that the hybrid oligosaccharide or polysaccharide comprises a hexose monosaccharide derivative at the reducing end of the first repeat unit in place of the hexose monosaccharide normally present at the reducing end of the first repeat of said Gram positive bacterial capsular saccharide.

### Host Cell

The present invention also provides a host cell comprising:

i) one or more nucleic acids that encode glycosyltransferase(s);
ii) a nucleic acid that encodes an oligosaccharyl transferase;
iii) a nucleic acid that encodes a modified pneumolysin protein of the invention; and optionally
iv) a nucleic acid that encodes a polymerase (e.g. wzy).

Host cells that can be used to produce the bioconjugates of the invention, include archea, prokaryotic host cells, and eukaryotic host cells. Exemplary prokaryotic host cells for use in production of the bioconjugates of the invention, without limitation, Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species, Bacillus species, and Clostridium species. In a specific embodiment, the host cell is E. coli.

In an embodiment, the host cells used to produce the bioconjugates of the invention are engineered to comprise heterologous nucleic acids, e.g. heterologous nucleic acids that encode one or more carrier proteins and/or heterologous nucleic acids that encode one or more proteins, e.g. genes encoding one or more proteins. In a specific embodiment, heterologous nucleic acids that encode proteins involved in glycosylation pathways (e.g. prokaryotic and/or eukaryotic glycosylation pathways) may be introduced into the host cells of the invention. Such nucleic acids may encode proteins including, without limitation, oligosaccharyl transferases, epimerases, flippases, polymerases, and/or glycosyltransferases. Heterologous nucleic acids (e.g. nucleic acids that encode carrier proteins and/or nucleic acids that encode other proteins, e.g. proteins involved in glycosylation) can be introduced into the host cells of the invention using methods such as electroporation, chemical transformation by heat shock, natural transformation, phage transduction, and conjugation. In specific embodiments, heterologous nucleic acids are introduced into the host cells of the invention using a plasmid, e.g. the heterologous nucleic acids are expressed in the host cells by a plasmid (e.g. an expression vector). In another specific embodiment, heterologous nucleic acids are introduced into the host cells of the invention using the method of insertion described in International Patent application No. PCT/EP2013/068737 (published as WO 14/037585).

Thus, the present invention also provides a host cell comprising:

i) one or more nucleic acids that encode glycosyltransferase(s);
ii) a nucleic acid that encodes an oligosaccharyl transferase;
iii) a nucleic acid that encodes a modified pneumolysin protein of the invention;
iv) a nucleic acid that encodes a polymerase (e.g. wzy); and
vi) a nucleic acid that encodes a flippase (e.g. wxy).

In an embodiment, additional modifications may be introduced (e.g. using recombinant techniques) into the host cells of the invention. For example, host cell nucleic acids (e.g. genes) that encode proteins that form part of a possibly competing or interfering glycosylation pathway (e.g. compete or interfere with one or more heterologous genes involved in glycosylation that are recombinantly introduced into the host cell) can be deleted or modified in the host cell background (genome) in a manner that makes them inactive/dysfunctional (i.e. the host cell nucleic acids that are deleted/modified do not encode a functional protein or do not encode a protein whatsoever). In an embodiment, when nucleic acids are deleted from the genome of the host cells of the invention, they are replaced by a desirable sequence, e.g. a sequence that is useful for glycoprotein production.

Exemplary genes that can be deleted in host cells (and, in some cases, replaced with other desired nucleic acid sequences) include genes of host cells involved in glycolipid biosynthesis, such as waaL (see, e.g. Feldman et al. 2005, PNAS USA 102:3016-3021), the lipid A core biosynthesis cluster (waa), galactose cluster (gal), arabinose cluster (ara), colonic acid cluster (wc), capsular polysaccharide cluster, undecaprenol-pyrophosphate biosynthesis genes (e.g. uppS (Undecaprenyl pyrophosphate synthase), uppP (Undecaprenyl diphosphatase)), Und-P recycling genes, metabolic enzymes involved in nucleotide activated sugar biosynthesis, enterobacterial common antigen cluster, and prophage O antigen modification clusters like the gtrABS cluster.

Such a modified prokaryotic host cell comprises nucleic acids encoding enzymes capable of producing a bioconjugate comprising an antigen, for example a saccharide antigen attached to a modified pneumolysin protein of the invention. Such host cells may naturally express nucleic acids specific for production of a saccharide antigen, or the host cells may be made to express such nucleic acids, i.e. in certain embodiments said nucleic acids are heterologous to the host cells. In certain embodiments, one or more of said nucleic acids specific for production of a saccharide antigen are heterologous to the host cell and intergrated into the genome of the host cell. In certain embodiments, the host cells of the invention comprise nucleic acids encoding additional enzymes active in the N-glycosylation of proteins, e.g. the host cells of the invention further comprise a nucleic acid encoding an oligosaccharyl transferase and/or one or more nucleic acids encoding other glycosyltransferases.

Nucleic acid sequences comprising capsular polysaccharide gene clusters can be inserted into the host cells of the invention. In a specific embodiment, the capsular polysaccharide gene cluster inserted into a host cell of the invention is a capsular polysaccharide gene cluster from an E. coli strain, a Streptococcus strain (e.g. S. pneumoniae, S. pyrogenes, S. agalacticae), a Staphylococcus strain (e.g. S. aureus), or a Burkholderia strain (e.g. B. mallei, B. pseudomallei, B. thailandensis). Disclosures of methods for making such host cells which are capable of producing bioconjugates are found in WO 06/119987, WO 09/104074, WO 11/62615, WO 11/138361, WO 14/57109, WO14/72405 and WO16/20499.

In an embodiment, the host cell comprises a nucleic acid that encodes a modified pneumolysin protein in a plasmid in the host cell.

### Glycosylation Machinery

The host cells of the invention comprise, and/or can be modified to comprise, nucleic acids that encode genetic machinery (e.g. glycosyltransferases, flippases, polymerases, and/or oligosaccharyltransferases) capable of producing hybrid oligosaccharides and/or polysaccharides, as well as genetic machinery capable of linking antigens to the modified pneumolysin protein of the invention.

### Glycosyltransferases

The host cells of the invention comprise nucleic acids that encode glycosyltransferases that produce an oligosaccharide or polysaccharide repeat unit. In an embodiment, said repeat unit does not comprise a hexose at the reducing end, and said oligosaccharide or polysaccharide repeat unit is derived from a donor oligosaccharide or polysaccharide repeat unit that comprises a hexose at the reducing end.

In an embodiment, the host cells of the invention may comprise a nucleic acid that encodes a glycosyltransferase that assembles a hexose monosaccharide derivative onto undecaprenyl pyrophosphate (Und-PP). In one aspect, the glycosyltransferase that assembles a hexose monosaccharide derivative onto Und-PP is heterologous to the host cell and/or heterologous to one or more of the genes that encode glycosyltransferase(s). Said glycosyltransferase can be derived from, e.g. Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species. In a specific embodiment, the glycosyltransferase that assembles a hexose monosaccharide derivative onto Und-PP is wecA, optionally from E. coli (wecA can assemble GlcNAc onto UndP from UDP-GlcNAc). In an embodiment, the hexose monosaccharide is selected from the group consisting of glucose, galactose, rhamnose, arabinotol, fucose and mannose (e.g. galactose).

In an embodiment, the host cells of the invention may comprise nucleic acids that encode one or more glycosyltransferases capable of adding a monosaccharide to the hexose monosaccharide derivative assembled on Und-PP (for example in the synthesis of CP33F).

In a specific embodiment, said one or more glycosyltransferases capable of adding a monosaccharide to the hexose monosaccharide derivative is the galactosyltransferase (wfeD) from Shigella boyedii. In another specific embodiment, said one or more glycosyltransferases capable of adding a monosaccharide to the hexose monosaccharide derivative is the galactofuranosyltransferase (wbeY) from E. coli O28. In an embodiment, said one or more glycosyltransferases capable of adding a monosaccharide to the hexose monosaccharide derivative comprise the galactofuranosyltransferase (wbeY) from E. coli O28 having an amino acid sequence of SEQ ID NO. 90 (GenBank: DQ462205.1) or an amino acid sequence at least 80%, 85%, 90%, 92%, 95%, 96%, 97%, 98% or 99% identical to SEQ ID NO. 90, for example comprising at least 100, 150 or 200 contiguous amino acids of the full length sequence. In another specific embodiment, said one or more glycosyltransferases capable of adding a monosaccharide to the hexose monosaccharide derivative is the galactofuranosyltransferase (wfdK) from E. coli O167. In an embodiment, said one or more glycosyltransferases capable of adding a monosaccharide to the hexose monosaccharide derivative comprise the galactofuranosyltransferase (wfdK) from E. coli O167 having an amino acid sequence of SEQ ID NO. 89 (GenBank: EU296408.1) or an amino acid sequence at least 80%, 85%, 90%, 92%, 95%, 96%, 97%, 98% or 99% identical to SEQ ID NO. 89, for example comprising at least 100, 150 or 200 contiguous amino acids of the full length sequence. Galf-transferases, such as wfdK and wbeY, can transfer Galf (Galactofuranose) from UDP-Galf to -GlcNAc-P-P-Undecaprenyl. In another specific embodiment, said one or more glycosyltransferases capable of adding a monosaccharide to the hexose monosaccharide derivative are the galactofuranosyltransferase (wbeY) from E. coli O28 and the galactofuranosyltransferase (wfdK) from E. coli O167.

In an embodiment, the host cells of the invention comprise nucleic acids that encode glycosyltransferases that assemble the donor oligosaccharide or polysaccharide repeat unit onto the hexose monosaccharide derivative.

In an embodiment, the glycosyltransferases that assemble the donor oligosaccharide or polysaccharide repeat unit onto the hexose monosaccharide derivative comprise a glycosyltransferase that is capable of adding the hexose monosaccharide present at the reducing end of the first repeat unit of the donor oligosaccharide or polysaccharide to the hexose monosaccharide derivative. Exemplary glycosyltransferases include galactosyltransferases (wclP), e.g. wclP from E. coli O21. In an embodiment, said one or more glycosyltransferases that assemble the donor oligosaccharide or polysaccharide repeat unit onto the hexose monosaccharide derivative comprise the galactosyltransferase (wclP) from E. coli O21 having an amino acid sequence of SEQ ID NO. 115 (GenBank: EU694098.1) or an amino acid sequence at least 80%, 85%, 90%, 92%, 95%, 96%, 97%, 98% or 99% identical to SEQ ID NO. 115, for example comprising at least 100, 150 or 200 contiguous amino acids of the full length sequence.

In one embodiment, the glycosyltransferases that assemble the donor oligosaccharide or polysaccharide repeat unit onto the hexose monosaccharide derivative comprise a glycosyltransferase that is capable of adding the monosaccharide that is adjacent to the hexose monosaccharide present at the reducing end of the first repeat unit of the donor oligosaccharide or polysaccharide to the hexose monosaccharide present at the reducing end of the first repeat unit of the donor oligosaccharide or polysaccharide. Exemplary glycosyltransferases include glucosyltransferase (wclQ), e.g. wclQ from E. coli O21. In an embodiment, said one or more glycosyltransferases that assemble the donor oligosaccharide or polysaccharide repeat unit onto the hexose monosaccharide derivative comprise the glucosyltransferase (wclQ) from E. coli O167 having an amino acid sequence of SEQ ID NO. 116 (GenBank: EU694098.1) or an amino acid sequence at least 80%, 85%, 90%, 92%, 95%, 96%, 97%, 98% or 99% identical to SEQ ID NO. 116, for example comprising at least 100, 150 or 200 contiguous amino acids of the full length sequence.

In an embodiment, a host cell of the invention comprises glycosyltransferases for synthesis of the repeat units of an oligosaccharide or polysaccharide selected from the following capsular polysaccharide gene clusters: S. pneumoniae CP1, CP2, CP3, CP4, CP5, CP6(A,B,C,D), CP7(A,B,C), CP8, CP9(A,L,N,V), CP10(A,B,C,F), CP11(A,B,C,D,F), CP12(A,B,F), CP13, CP14 CP15(A,B,C,F), CP16(A,F), CP17(A,F), CP18(A,B,C,F), CP19(A,B,C,F), CP20, CP21, CP22(A,F), CP23(A,B,F), CP24(A,B,F), CP25(A,F), CP26, CP27,CP28(A,F), CP29, CP31, CP32(A,F), CP33(A,B,C,D,F), CP34, CP35(A,B,C,D,F), CP36, CP37, CP38, CP39, CP40, CP41(A,F), CP42, CP43, CP44, CP45, CP46, CP47(A,F), or CP48. The capsular biosynthetic genes of S. pneumoniae are described in Bentley et al. (PLoS Genet. 2006 March; 2(3): e31 and the sequences are provided in GenBank. In a specific embodiment, the glycosyltransferases for synthesis of the repeat units of an oligosaccharide or polysaccharide are selected from the following capsular polysaccharide gene clusters: CP4, CP8, CP12F, CP15A, CP16F, CP22F, CP23A, CP24F, CP31, CP33F, CP35B, or CP38. In a specific embodiment, the glycosyltransferases for synthesis of the repeat units of an oligosaccharide or polysaccharide are selected from the following capsular polysaccharide gene clusters: CP4, CP12F or CP33F.

The capsular polysaccharide gene cluster maps between dexB and aliA in the pneumococcal chromosome (Llull et al., 1999, J. Exp. Med. 190, 241-251). There are typically four relatively conserved genes: (wzg), (wzh), (wzd), (wze) at the 5′ end of the capsular polysaccharide gene cluster (Jiang et al., 2001, Infect. Immun. 69, 1244-1255). Also included in the capsular polysaccharide gene cluster of S. pneumoniae are wzx (polysaccharide flippase gene) and wzy (polysaccharide polymerase gene). The CP gene clusters of all 90 S. pneumoniae serotypes have been sequenced by Sanger Institute (http://www.sanger.ac.uk/Projects/S_pneumoniae/CPS/), and wzx and wzy of 89 serotypes have been annotated and analyzed (Kong et al., 2005, J. Med. Microbiol. 54, 351-356). The capsular biosynthetic genes of S. pneumoniae are further described in Bentley et al. (PLoS Genet. 2006 March; 2(3): e31 and the sequences are provided in GenBank.

In an embodiment, a host cell of the invention comprises glycosyltransferases sufficient for synthesis of the repeat units of the CP4 saccharide comprising wzg, wzh, wzd and/or wze from S. pneumoniae CP4. In an embodiment, said one or more glycosyltransferases sufficient for synthesis of the repeat units of the CP4 saccharide comprising wzg, wzh, wzd and/or wze from S. pneumoniae CP4 having amino acid sequences of SEQ ID NOs. 91, 92, 93 and 94 respectively (GenBank: CR931635.1) or amino acid sequences at least 80%, 85%, 90%, 92%, 95%, 96%, 97%, 98% or 99% identical to SEQ ID NOs. 91, 92, 93 and 94, for example comprising at least 100, 150 or 200 contiguous amino acids of the full length sequence. Optionally the host cell of the invention also comprises wciI, wciJ, wciK, wciL, wzy, wciM, wzx, mnaA, fnlA, fnlB and fnlC from S. pneumoniae CP4. In an embodiment, the host cell of the invention comprises nucleic acid sequence encoding wciI, wciJ, wciK, wciL, wzy, wciM, wzx, mnaA, fnlA (also called “fnl1”), fnlB (also called “fnl2”) and fnlC (also called “fnl3”) from S. pneumoniae CP4 having amino acid sequences of SEQ ID NOs. 95, 96, 97, 98, 99, 100, 101, 102, 103, 104 and 105 respectively (GenBank: CR931635.1) or amino acid sequences at least 80%, 85%, 90%, 92%, 95%, 96%, 97%, 98% or 99% identical to SEQ ID NOs. 95, 96, 97, 98, 99, 100, 101, 102, 103, 104 and 105, for example comprising at least 100, 150 or 200 contiguous amino acids of the full length sequence. WciI is a predicted glycosyl-phosphate transferase and thus responsible for Und-PP-D-GalNAc synthesis, wciJ, wciK, and wciL are D-ManNAc, L-FucNAc, and D-Gal transferases, and wciM is a homolog of pyruvate transferases (Jiang S M, Wang L, Reeves P R: Molecular characterization of Streptococcus pneumoniae type 4, 6B, 8, and 18C capsular polysaccharide gene clusters. Infect Immun 2001, 69(3):1244-1255). Further details on the synthesis of CP4 can be found in WO2014/072405A1.

In an embodiment, a host cell of the invention comprises glycosyltransferases sufficient for synthesis of the repeat units of CP12F saccharide comprising wzg, wzh, wzd and/or wze from S. pneumoniae CP12F. In an embodiment, said one or more glycosyltransferases sufficient for synthesis of the repeat units of the CP12F saccharide comprising wzg, wzh, wzd and/or wze from S. pneumoniae CP12F having amino acid sequences of SEQ ID NOs. 106, 107, 108 and 109 respectively (GenBank: CR931660.1) or amino acid sequences at least 80%, 85%, 90%, 92%, 95%, 96%, 97%, 98% or 99% identical to SEQ ID NOs. 106, 107, 108 and 109, for example comprising at least 100, 150 or 200 contiguous amino acids of the full length sequence.

In an embodiment, a host cell of the invention comprises glycosyltransferases sufficient for synthesis of the repeat units of the donor oligosaccharide or polysaccharide comprising wciC, wciD, wciE, and/or wciF from S. pneumoniae CP33F. In an embodiment, said one or more glycosyltransferases sufficient for synthesis of the repeat units of the CP12F saccharide comprising wciC, wciD, wciE, and/or wciF from S. pneumoniae CP33F having amino acid sequences of SEQ ID NOs. 110, 111, 112 and 113 respectively (GenBank: CR931702.1) or amino acid sequences at least 80%, 85%, 90%, 92%, 95%, 96%, 97%, 98% or 99% identical to SEQ ID NOs. 110, 111, 112 and 113, for example comprising at least 100, 150 or 200 contiguous amino acids of the full length sequence. Optionally a host cell of the invention also comprises wchA (a Glc-1-P transferase) and/or wciB (a Galf transferase) from S. pneumoniae CP33F. In an embodiment, a host cell of the invention comprises nucleic acid sequence(s) encoding wchA (a Glc-1-P transferase) and/or wciB (a Galf transferase) from S. pneumoniae CP33F having amino acid sequences of SEQ ID NOs. 114 and 115 respectively (GenBank: CR931702.1) or amino acid sequences at least 80%, 85%, 90%, 92%, 95%, 96%, 97%, 98% or 99% identical to SEQ ID NOs. 114 and 115, for example comprising at least 100, 150 or 200 contiguous amino acids of the full length sequence. Suitably, said host cell is capable of producing a hybrid oligosaccharide or polysaccharide, wherein said hybrid oligosaccharide or polysaccharide is identical to S. pneumoniae CP33F, with the exception of the fact that said hybrid oligosaccharide or polysaccharide comprises a hexose monosaccharide derivative at the reducing end of the first repeat unit in place of the hexose monosaccharide normally present at the reducing end of the first repeat unit of S. pneumoniae CP33F.

In an embodiment, a host cell of the invention comprises glycosyltransferases that assemble the donor oligosaccharide or polysaccharide repeat unit onto the hexose monosaccharide derivative comprise a glycosyltransferase that is capable of adding the hexose monosaccharide present at the reducing end of the first repeat unit of the donor oligosaccharide or polysaccharide to the hexose monosaccharide derivative.

The host cell may further comprise additional genes for the synthesis of saccharides. For example, the host cell may comprise the cluster encoding CP type 4 from S. pneumoniae comprises genes wzg to fnlC (i.e. one or more or all of the genes wzg, wzh, wzd, wze, wciI, wciJ, wciK, wciL, wzy, wciM, wzx, mnaA, fnlA, fnlB, fnlC). UDP-D-FucNAc and UDP-D-ManNAc are made by the proteins mnaA, fnlA, fnlB, and fnlC. As described above, WciI is a predicted glycosyl-phosphate transferase and thus responsible for Und-PP-D-GalNAc synthesis, wciJ, wciK, and wciL are D-ManNAc, L-FucNAc, and D-Gal transferases, and wciM is a homolog of pyruvate transferases. These genes may be further components of the host cell.

For example, to synthesize a glycoengineered CP33F subunit (a repeat unit comprising a hexose monosaccharide derivative at the reducing end), two galactofuranosyltransferases, WbeY from E. coli O28 and WfdK from E. coli O167 may be used. GlcNAc may be assembled on UndP from UDP-GlcNAc by WecA (which exists in all Gram-negative bacteria that synthesize ECA and Gram-positive bacteria that makes Teichoic acid) (Annu Rev Microbiol. 2013; 67:313-36; Glycobiology. 2011 February; 21(2):138-51) to make a β (1,3) linkage. Thereafter, using the glycosyltransferases WciC, WciD, WciE and WciF from the S. pneumoniae CP33F gene cluster, the CP33F engineered subunit (β-D-Galf-1,3-β-D-Gal-1,3-α-D-Gal(α1,2-D-Gal)-1,3-β-D-Galf-(2Ac)-1,3-D-GlcNAc-PP-Undd) is synthesized. Thus, these genes may be further components of the host cell. In an embodiment, a plasmid may be used to produce the CP33F engineered subunit in the cytoplasm, from which it may be translocated into the periplasm by the flippase of CP33F where the wild type polysaccharide can be assembled on it by action of CP33F polymerase (wzy). The plasmid may also contain CP33F polymerase (wzy) and E. coli 016 galE and glf to enhance productivity of wild type polymerase. Further details on the synthesis of CP33F can be found in WO2016/020499A2.

### Oligosaccharyl Transferases

N-linked protein glycosylation—the addition of carbohydrate molecules to an asparagine residue in the polypeptide chain of the target protein—is the most common type of post-translational modification occurring in the endoplasmic reticulum of eukaryotic organisms. The process is accomplished by the enzymatic oligosaccharyltransferase complex (OST) responsible for the transfer of a preassembled oligosaccharide from a lipid carrier (dolichol phosphate) to an asparagine residue of a nascent protein within the conserved sequence Asn-X-Ser/Thr (where X is any amino acid except proline) in the Endoplasmic reticulum.

It has been shown that a bacterium, the food-borne pathogen Campylobacter jejuni, can also N-glycosylate its proteins (Wacker et al. Science. 2002; 298(5599):1790-3) due to the fact that it possesses its own glycosylation machinery. The machinery responsible of this reaction is encoded by a cluster called “pgl” (for protein glycosylation).

The C. jejuni glycosylation machinery can be transferred to E. coli to allow for the glycosylation of recombinant proteins expressed by the E. coli cells. Previous studies have demonstrated how to generate E. coli strains that can perform N-glycosylation (see, e.g. Wacker et al. Science. 2002; 298 (5599):1790-3; Nita-Lazar et al. Glycobiology. 2005; 15(4):361-7; Feldman et al. Proc Natl Acad Sci USA. 2005; 102(8):3016-21; Kowarik et al. EMBO J. 2006; 25(9):1957-66; Wacker et al. Proc Natl Acad Sci USA. 2006; 103(18):7088-93; International Patent Application Publication Nos. WO2003/074687, WO2006/119987, WO 2009/104074, and WO/2011/06261, and WO2011/138361).

Oligosaccharyl transferases transfer lipid-linked oligosaccharides to asparagine residues of nascent polypeptide chains that comprise a N-glycosylation consensus motif, e.g. Asn-X-Ser(Thr), wherein X can be any amino acid except Pro; or Asp(Glu)-X-Asn-Z-Ser(Thr), wherein X and Z are independently selected from any natural amino acid except Pro (see WO 2006/119987). See, e.g. WO 2003/074687 and WO 2006/119987, the disclosures of which are herein incorporated by reference in their entirety.

In an embodiment, the host cells of the invention comprise a nucleic acid that encodes an oligosaccharyl transferase. The nucleic acid that encodes an oligosaccharyl transferase can be native to the host cell, or can be introduced into the host cell using genetic approaches, as described above. In a specific embodiment, the oligosaccharyl transferase is an oligosaccharyl transferase from Campylobacter. In another specific embodiment, the oligosaccharyl transferase is an oligosaccharyl transferase from Campylobacter jejuni (i.e. pglB; see, e.g. Wacker et al. 2002, Science 298:1790-1793; see also, e.g. NCBI Gene ID: 3231775, UniProt Accession No. 086154). In another specific embodiment, the oligosaccharyl transferase is an oligosaccharyl transferase from Campylobacter lari (see, e.g. NCBI Gene ID: 7410986).

In a specific embodiment, the host cells of the invention comprise a nucleic acid sequence encoding an oligosaccharyl transferase, wherein said nucleic acid sequence encoding an oligosaccharyl transferase (e.g. pglB from Campylobacter jejuni) is integrated into the genome of the host cell. In an embodiment, a host cell of the invention comprises a nucleic acid sequence encoding an oligosaccharyl transferase having an amino acid sequence of SEQ ID NO. 120 (GenBank: AF108897.1) or an amino acid sequence at least 80%, 85%, 90%, 92%, 95%, 96%, 97%, 98% or 99% identical to SEQ ID NO. 120, for example comprising at least 100, 150 or 200 contiguous amino acids of the full length sequence.

In another specific embodiment, provided herein is a modified prokaryotic host cell comprising (i) a glycosyltransferase derived from an capsular polysaccharide cluster from S. pneumoniae, wherein said glycosyltransferase is integrated into the genome of said host cell; (ii) a nucleic acid encoding an oligosaccharyl transferase (e.g. pglB from Campylobacter jejuni), wherein said nucleic acid encoding an oligosaccharyl transferase is integrated into the genome of the host cell; and (iii) a modified pneumolysin protein of the invention, wherein said modified pneumolysin protein is either plasmid-borne or integrated into the genome of the host cell. There is also provided a method of making a modified prokaryotic host cell comprising (i) integrating a glycosyltransferase derived from an capsular polysaccharide cluster from S. pneumoniae into the genome of said host cell; (ii) integrating a nucleic acid encoding an oligosaccharyl transferase (e.g. pglB from Campylobacter jejuni) into the genome of the host cell; and (iii) integrating into a host cell a modified pneumolysin protein of the invention either plasmid-borne or integrated into the genome of the host cell.

In specific embodiment is a host cell of the invention, wherein at least one gene of the host cell has been functionally inactivated or deleted, optionally wherein the waaL gene of the host cell has been functionally inactivated or deleted, optionally wherein the waaL gene of the host cell has been replaced by a nucleic acid encoding an oligosaccharyltransferase, optionally wherein the waaL gene of the host cell has been replaced by C. jejuni pglB.

### Polymerases

In an embodiment, a polymerase (e.g. wzy) is introduced into a host cell of the invention (i.e. the polymerase is heterologous to the host cell). In an embodiment, the polymerase is a bacterial polymerase. In an embodiment, the polymerase is a capsular polysaccharide polymerase (e.g. wzy) or an O antigen polymerase (e.g. wzy). In an embodiment, the polymerase is a capsular polysaccharide polymerase (e.g. wzy).

In an embodiment, a polymerase of a capsular polysaccharide biosynthetic pathway is introduced into a host cell of the invention.

In another specific embodiment, a polymerase of a capsular polysaccharide biosynthetic pathway of S. pneumoniae is introduced into a host cell of the invention.

In an embodiment, the polymerase introduced into the host cells of the invention is the wzy gene from a capsular polysaccharide gene cluster of S. pneumoniae CP1, CP2, CP4, CP5, CP6(A,B,C,D), CP7 (A, B, C), CP8, CP9(A,L,N,V), CP10(A,B,C,F), CP11(A, B, C, D, F), CP12(A,B,F), CP13, CP14 CP15(A,B,C,F), CP16(A,F), CP17(A,F), CP18(A,B,C,F), CP19(A,B,C,F), CP20, CP21, CP22(A,F), CP23(A,B,F), CP24(A,B,F), CP25(A,F), CP26, CP27,CP28(A,F), CP29, CP31, CP32(A,F), CP33(A,B,C,D,F), CP34, CP35(A,B,C,D,F), CP36, CP38, CP39, CP40, CP41(A,F), CP42, CP43, CP44, CP45, CP46, CP47(A,F) or CP48. In a specific embodiment, the polymerase introduced into the host cells of the invention is the wzy gene from a capsular polysaccharide gene cluster of CP4, CP8, CP12F, CP15A, CP16F, CP22F, CP23A, CP24F, CP31, CP33F, CP35B, or CP38. In a specific embodiment, the polymerase introduced into the host cells of the invention is the wzy gene from a capsular polysaccharide gene cluster of CP4, CP12F or CP33F. For example, a host cell of the invention may comprise a nucleic acid sequence encoding a wzy polymerase from a capsular polysaccharide gene cluster of CP4, CP12F or CP33F having an amino acid sequence as provided in GenBank: CR931635.1, GenBank: CR931660.1 and GenBank: CR931702.1 or an amino acid sequence at least 80%, 85%, 90%, 92%, 95%, 96%, 97%, 98% or 99% identical thereto, for example comprising at least 100, 150 or 200 contiguous amino acids of the full length sequence.

Other polymerases that can introduced into the host cells of the invention are from S. pneumoniae described in Bentley S D, Aanensen D M, Mavroidi A, Saunders D, Rabbinowitsch E, Collins M, Donohoe K, Harris D, Murphy L, Quail M A et al: Genetic analysis of the capsular biosynthetic locus from all 90 pneumococcal serotypes. PLoS genetics 2006, 2(3):e31).

In another specific embodiment, said wzy polymerase is incorporated (e.g. inserted into the genome of or plasmid expressed by) in said host cell as part of a S. pneumoniae capsular polysaccharide cluster, wherein said S. pneumoniae capsular polysaccharide cluster has been modified to comprise the wzy polymerase.

In a specific embodiment, a nucleic acid sequence encoding the S. pneumoniae wzy polymerase is inserted into or expressed by the host cells of the invention. Thus, a host cell of the invention may further comprise an S. pneumoniae wzy polymerase.

### Flippases

In an embodiment, a flippase (wzx) is introduced into a host cell of the invention (i.e. the flippase is heterologous to the host cell). Thus, a host cell of the invention may further comprise a flippase. In an embodiment, the flippase is a bacterial flippase. Flippases translocate wild type repeating units and/or their corresponding engineered (hybrid) repeat units from the cytoplasm into the periplam of host cells (e.g. E. coli). Thus, a host cell of the invention may comprise a nucleic acid that encodes a flippase (wzx).

In a specific embodiment, a flippase of a capsular polysaccharide biosynthetic pathway is introduced into a host cell of the invention.

In another specific embodiment, a flippase of a capsular polysaccharide biosynthetic pathway of S. pneumoniae is introduced into a host cell of the invention. In certain embodiments, the flippase introduced into the host cells of the invention is the wzx gene from a capsular polysaccharide gene cluster of S. pneumoniae CP1, CP2, CP4, CP5, CP6(A,B,C,D), CP7(A,B,C), CP8, CP9(A,L,N,V), CP10(A,B,C,F), CP11(A,B,C,D,F), CP12(A,B,F), CP13, CP14 CP15(A,B,C,F), CP16(A,F), CP17(A,F), CP18(A,B,C,F), CP19(A,B,C,F), CP20, CP21, CP22(A,F), CP23(A,B,F), CP24(A,B,F), CP25(A,F), CP26, CP27, CP28(A,F), CP29, CP31, CP32(A,F), CP33(A,B,C,D,F), CP34, CP35(A,B,C,D,F), CP36, CP38, CP39, CP40, CP41(A,F), CP42, CP43, CP44, CP45, CP46, CP47(A,F), or CP48. In a specific embodiment, the flippase introduced into the host cells of the invention is the wzx gene from a capsular polysaccharide gene cluster of CP4, CP8, CP12F, CP15A, CP16F, CP22F, CP23A, CP24F, CP31, CP33F, CP35B, or CP38. For example, a host cell of the invention may comprise a nucleic acid sequence encoding a wzx flippase from a capsular polysaccharide gene cluster of CP4, CP12F or CP33F having an amino acid sequence as provided in GenBank: CR931635.1, GenBank: CR931660.1 and GenBank: CR931702.1 or an amino acid sequence at least 80%, 85%, 90%, 92%, 95%, 96%, 97%, 98% or 99% identical thereto, for example comprising at least 100, 150 or 200 contiguous amino acids of the full length sequence.

In a specific embodiment, the flippase introduced into the host cells of the invention is the wzx gene from a capsular polysaccharide gene cluster of CP4, CP12F or CP33F.

Other flippases that can introduced into the host cells of the invention are from S. pneumoniae described in Bentley S D, Aanensen D M, Mavroidi A, Saunders D, Rabbinowitsch E, Collins M, Donohoe K, Harris D, Murphy L, Quail M A et al. “Genetic analysis of the capsular biosynthetic locus from all 90 pneumococcal serotypes” PLoS genetics 2006, 2(3):e31).

Other flippases that can be introduced into the host cells of the invention are for example from Campylobacter jejuni (e.g. pglK).

Enzymes that Modify Monosaccharides

### Accessory Enzymes

In an embodiment, nucleic acids encoding one or more accessory enzymes are introduced into the host cells of the invention. Thus, a host cell of the invention may further comprise one or more of these accessory enzymes. Such nucleic acids encoding one or more accessory enzymes can be either plasmid-borne or integrated into the genome of the host cells of the invention. Exemplary accessory enzymes include, without limitation, epimerases, branching, modifying (e.g. to add cholins, glycerolphosphates, pyruvates), amidating, chain length regulating, acetylating, formylating, polymerizing enzymes.

In certain embodiments, enzymes that are capable of modifying monosaccharides are introduced into a host cell of the invention (i.e. the enzymes that are capable of modifying monosaccharides are heterologous to the host cell). Such enzymes include, e.g. epimerases and racemases. Thus, a host cell of the invention may further comprise an epimerase and/or racemase.

In an embodiment, the epimerases and racemases are from bacteria. In certain embodiments, the epimerases and/or racemases introduced into the host cells of the invention are from Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species.

In certain embodiments, the epimerase inserted into a host cell of the invention is an epimerase described in International Patent Application Publication No. WO2011/062615, the disclosure of which is incorporated by reference herein in its entirety. In one embodiment, the epimerase is the epimerase encoded by the Z3206 gene of E. coli strain O157. The Z3206 epimerase converts GlcNAc-UndPP (product of E. coli wecA) to GalNAc-UndPP (Rush J S, Alaimo C, Robbiani R, Wacker M, Waechter C J. J Biol Chem. 2010 Jan. 15; 285(3):1671-80). In an embodiment, a host cell of the invention comprises an epimerase having an amino acid sequence of SEQ ID NO. 118 ([Escherichia coli O157:H7 str. EDL933] GenBank: AAG57102.1) or an amino acid sequence at least 80%, 85%, 90%, 92%, 95%, 96%, 97%, 98% or 99% identical to SEQ ID NO. 118, for example comprising at least 100, 150 or 200 contiguous amino acids of the full length sequence. See, e.g. WO 2011/062615 and Rush et al. 2009, The Journal of Biological Chemistry 285:1671-1680, which is incorporated by reference herein in its entirety. In another embodiment, the epimerase is galE (UPD-Galactose epimerase). Z3206 and galE convert GlcNAc-P-P-undecaprenyl to GalNAc-P-P-undecaprenyl. In an embodiment, a host cell of the invention comprises an epimerase having an amino acid sequence of SEQ ID NO. 119 (UniProtKB—P09147) or an amino acid sequence at least 80%, 85%, 90%, 92%, 95%, 96%, 97%, 98% or 99% identical to SEQ ID NO. 119, for example comprising at least 100, 150 or 200 contiguous amino acids of the full length sequence. In another embodiment, the epimerase is UDP-GlcNAc/Glc 4-Epimerase (gne) from Campylobacter jejuni. In a specific embodiment, the host cells of the invention comprise a nucleic acid sequence encoding an epimerase, wherein said nucleic acid sequence encoding an epimerase is integrated into the genome of the host cell.

In an embodiment, a host cell of the invention further comprises a mutase, for example glf (UDP-galactopyranose mutase).

In an embodiment, a host cell of the invention further comprises RcsA (an activator of CP synthesis). RcsA is an unstable positive regulator required for the synthesis of colanic acid capsular polysaccharide in Escherichia coli.

### Genetic Background

Exemplary host cells that can be used to generate the host cells of the invention include, without limitation, Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species, Bacillus species, and Clostridium species. In a specific embodiment, the host cell used herein is E. coli.

In an embodiment, the host cell genetic background is modified by, e.g. deletion of one or more genes. Exemplary genes that can be deleted in host cells (and, in some cases, replaced with other desired nucleic acid sequences) include genes of host cells involved in glycolipid biosynthesis, such as waaL (see, e.g. Feldman et al. 2005, PNAS USA 102:3016-3021), the O antigen cluster (rfb or wb), enterobacterial common antigen cluster (wec), the lipid A core biosynthesis cluster (waa), and prophage O antigen modification clusters like the gtrABS cluster. In a specific embodiment, one or more of the waaL gene, gtrA gene, gtrB gene, gtrS gene, or a gene or genes from the wec cluster or a gene or genes from the rfb gene cluster are deleted or functionally inactivated from the genome of a prokaryotic host cell of the invention. In one embodiment, a host cell used herein is E. coli, wherein the waaL gene, gtrA gene, gtrB gene, gtrS gene are deleted or functionally inactivated from the genome of the host cell. In another embodiment, a host cell used herein is E. coli, wherein the waaL gene and gtrS gene are deleted or functionally inactivated from the genome of the host cell. In another embodiment, a host cell used herein is E. coli, wherein the waaL gene and genes from the wec cluster are deleted or functionally inactivated from the genome of the host cell.

### Benefits

The host cells of the invention are of particular commercial importance and relevance, as they allow for large scale fermentation of bioconjugates comprising saccharide, for example, Streptococcus antigens that can be used as therapeutics (e.g. in immunogenic compositions, vaccines), at a lower risk due to the increased stability of the chromosomally inserted DNA and thus expression of the DNA of interest during fermentation. The host cells of the invention are advantageous over host cells that rely on plasmid borne expression of nucleic acids required for generation of the bioconjugates of the invention because, inter alia, antibiotic selection during fermentation is not required once the heterologous DNA is inserted into the host cell genome. That is, when the insert DNA is inserted in the chromosome, it doesn't need to be selected for, because it is propagated along with replication of the host genome. Further, it is a disadvantage in plasmid borne systems that with every generation (i.e. cycle of host cell replication) the risk for losing the plasmid increases. This loss of plasmid is due to the sometimes inappropriate distribution of plasmids to daughter cells at the stage of cell separation during cell division. At large scale, bacterial cell cultures duplicate more often than in smaller fermentation scales to reach high cell densities. Thus, higher cell stability and insert DNA expression leads to higher product yields, providing a distinct advantage. Cell stability is furthermore a process acceptance criteria for approval by regulatory authorities, while antibiotic selection is generally not desired during fermentation for various reasons, e.g. antibiotics present as impurities in the final medical products and bear the risk of causing allergic reactions, and antibiotics may promote antibiotic resistance (e.g. by gene transfer or selection of resistant pathogens).

The present application provides host cells for use in making bioconjugates comprising saccharide antigens that can be used as therapeutics (e.g. in immunogenic compositions, vaccines), wherein certain genetic elements required to drive the production of bioconjugates are integrated stably into the host cell genome. Consequently the host cell can contain a reduced number of plasmids, just a single plasmid or no plasmids at all. In some embodiments, the presence of a single plasmid can result in greater flexibility of the production strain and the ability to change the nature of the conjugation (in terms of its saccharide or carrier protein content) easily leading to greater flexibility of the production strain.

In general, a reduction in the use of plasmids leads to a production strain which is more suited for use in the production of medicinal products. A drawback of essential genetic material being present on plasmids is the requirement for selection pressure to maintain the episomal elements in the host cell. The selection pressure requires the use of antibiotics, which is undesirable for the production of medicinal products due to, e.g. the danger of allergic reactions against the antibiotics and the additional costs of manufacturing.

Furthermore, selection pressure is often not complete, resulting in inhomogeneous bacterial cultures in which some clones have lost the plasmid and thus are not producing the bioconjugate. The host cells of the invention therefore are able to produce a safer product that can be obtained in high yields.

### Bioconjugates

The host cells of the invention can be used to produce bioconjugates comprising a saccharide antigen, for example a Streptococcus pneumoniae antigen linked to a modified pneumolysin protein of the invention. Methods of producing bioconjugates using host cells are described for example in WO 2003/074687 and WO 2006/119987. Bioconjugates, as described herein, have advantageous properties over chemical conjugates of antigen-carrier protein, in that they require less chemicals in manufacture and are more consistent in terms of the final product generated.

In an embodiment, provided herein is a bioconjugate comprising a modified pneumolysin protein linked to a Streptococcus pneumoniae antigen. In a specific embodiment, said Streptococcus pneumoniae antigen is a capsular saccharide (e.g. capsular polysaccharide). In a specific embodiment, provided herein is a bioconjugate comprising a modified pneumolysin protein of the invention and an antigen selected from a capsular saccharide (e.g. capsular polysaccharide) of Streptococcus pneumoniae serotype CP1, CP2, CP3, CP4, CP5, CP6(A,B,C,D), CP7(A,B,C), CP8, CP9(A,L,N,V), CP10(A,B,C,F), CP11(A,B,C,D,F), CP12(A,B,F), CP13, CP14 CP15(A,B,C,F), CP16(A,F), CP17(A,F), CP18(A,B,C,F), CP19(A,B,C,F), CP20, CP21, CP22(A,F), CP23(A,B,F), CP24(A,B,F), CP25(A,F), CP26, CP27, CP28(A,F), CP29, CP31, CP32(A,F), CP33(A,B,C,D,F), CP34, CP35(A,B,C,D,F), CP36, CP37, CP38, CP39, CP40, CP41(A,F), CP42, CP43, CP44, CP45, CP46, CP47(A,F), or CP48. In a specific embodiment, provided herein is a bioconjugate comprising a modified pneumolysin protein of the invention and an antigen selected from a capsular saccharide (e.g. capsular polysaccharide) of Streptococcus pneumoniae serotype CP4, CP8, CP12F, CP15A, CP16F, CP22F, CP23A, CP24F, CP31, CP33F, CP35B, or CP38. In a specific embodiment, provided herein is a bioconjugate comprising a modified pneumolysin protein of the invention and an antigen selected from a capsular saccharide (e.g. capsular polysaccharide) of Streptococcus pneumoniae serotype 4, 12F or 33F.

The bioconjugates of the invention can be purified for example, by chromatography (e.g. ion exchange, anionic exchange, affinity, and sizing column chromatography), centrifugation, differential solubility, or by any other standard technique for the purification of proteins. See, e.g. Saraswat et al. 2013, Biomed. Res. Int. ID #312709 (p. 1-18); see also the methods described in WO 2009/104074. Further, the bioconjugates may be fused to heterologous polypeptide sequences described herein or otherwise known in the art to facilitate purification. The actual conditions used to purify a particular bioconjugate will depend, in part, on the synthesis strategy and on factors such as net charge, hydrophobicity, and/or hydrophilicity of the bioconjugate, and will be apparent to those having skill in the art.

A further aspect of the invention is a process for producing a bioconjugate that comprises (or consists of) a modified pneumolysin protein linked to a saccharide, said method comprising (i) culturing the host cell of the invention under conditions suitable for the production of proteins (and optionally under conditions suitable for the production of saccharides) and (ii) isolating the bioconjugate produced by said host cell.

A further aspect of the invention is a bioconjugate produced by the process of the invention, wherein said bioconjugate comprises a saccharide linked to a modified pneumolysin protein.

### Analytical Methods

Various methods can be used to analyze the structural compositions and sugar chain lengths of the bioconjugates of the invention.

In one embodiment, hydrazinolysis can be used to analyze glycans. First, polysaccharides are released from their protein carriers by incubation with hydrazine according to the manufacturer's instructions (Ludger Liberate Hydrazinolysis Glycan Release Kit, Oxfordshire, UK). The nucleophile hydrazine attacks the glycosidic bond between the polysaccharide and the carrier protein and allows release of the attached glycans. N-acetyl groups are lost during this treatment and have to be reconstituted by re-N-acetylation. The free glycans are purified on carbon columns and subsequently labeled at the reducing end with the fluorophor 2-amino benzamide. See Bigge J C, Patel T P, Bruce J A, Goulding P N, Charles S M, Parekh R B: Nonselective and efficient fluorescent labeling of glycans using 2-amino benzamide and anthranilic acid. Anal Biochem 1995, 230(2):229-238. The labeled polysaccharides are separated on a GlycoSeptember-N column (GL Sciences) according to the HPLC protocol of Royle et al. See Royle L, Mattu T S, Hart E, Langridge J I, Merry A H, Murphy N, Harvey D J, Dwek R A, Rudd P M: An analytical and structural database provides a strategy for sequencing O-glycans from microgram quantities of glycoproteins. Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit could be confirmed and additionally in homogeneity of the polysaccharide composition could be identified.

In another embodiment, SDS-PAGE or capillary gel electrophoresis can be used to assess glycans and bioconjugates. Polymer length for the O antigen glycans is defined by the number of repeat units that are linearly assembled. This means that the typical ladder like pattern is a consequence of different repeat unit numbers that compose the glycan. Thus, two bands next to each other in SDS PAGE or other techniques that separate by size differ by only a single repeat unit. These discrete differences are exploited when analyzing glycoproteins for glycan size: The unglycosylated carrier protein and the bioconjugate with different polymer chain lengths separate according to their electrophoretic mobilities. The first detectable repeating unit number (n₁) and the average repeating unit number (n_{average}) present on a bioconjugate are measured. These parameters can be used to demonstrate batch to batch consistency or polysaccharide stability.

In another embodiment, high mass MS and size exclusion HPLC could be applied to measure the size of the complete bioconjugates.

In another embodiment, an anthrone-sulfuric acid assay can be used to measure polysaccharide yields. See Leyva A, Quintana A, Sanchez M, Rodriguez E N, Cremata J, Sanchez J C: Rapid and sensitive anthrone-sulfuric acid assay in microplate format to quantify carbohydrate in biopharmaceutical products: method development and validation. Biologicals: journal of the International Association of Biological Standardization 2008, 36(2):134-141. In another embodiment, a Methylpentose assay can be used to measure polysaccharide yields. See, e.g. Dische et al. J Biol Chem. 1948 September; 175(2):595-603.

### Change in Glycosylation Site Usage

To show that the site usage in a specific protein is changed in a multiple plasmid system as opposed to an inserted system, the glycosylation site usage must be quantified. Methods to do so are listed below.

Glycopeptide LC-MS/MS: bioconjugates are digested with protease(s), and the peptides are separated by a suitable chromatographic method (C18, Hydrophilic interaction HPLC HILIC, GlycoSepN columns, SE HPLC, AE HPLC), and the different peptides are identified using MS/MS. This method can be used with our without previous sugar chain shortening by chemical (smith degradation) or enzymatic methods. Quantification of glycopeptide peaks using UV detection at 215 to 280 nm allow relative determination of glycosylation site usage.

Size exclusion HPLC: Higher glycosylation site usage is reflected by a earlier elution time from a SE HPLC column.

### Homogeneity

Bioconjugate homogeneity (i.e. the homogeneity of the attached sugar residues) can be assessed using methods that measure glycan length and hydrodynamic radius.

### Analytical Methods for Testing Benefit

### Yield.

Yield is measured as carbohydrate amount derived from a liter of bacterial production culture grown in a bioreactor under controlled and optimized conditions. After purification of bioconjugate, the carbohydrate yields can be directly measured by either the anthrone assay or ELISA using carbohydrate specific antisera. Indirect measurements are possible by using the protein amount (measured by BCA, Lowry, or bardford assays) and the glycan length and structure to calculate a theoretical carbohydrate amount per gram of protein. In addition, yield can also be measured by drying the glycoprotein preparation from a volatile buffer and using a balance to measure the weight.

### Homogeneity.

Homogeneity means the variability of glycan length and possibly the number of glycosylation sites. Methods listed above can be used for this purpose. SE-HPLC allows the measurement of the hydrodynamic radius. Higher numbers of glycosylation sites in the carrier lead to higher variation in hydrodynamic radius compared to a carrier with less glycosylation sites. However, when single glycan chains are analyzed, they may be more homogenous due to the more controlled length. Glycan length is measured by hydrazinolysis, SDS PAGE, and CGE. In addition, homogeneity can also mean that certain glycosylation site usage patterns change to a broader/narrower range. These factors can be measured by Glycopeptide LC-MS/MS.

### Strain Stability and Reproducibility.

Strain stability during bacterial fermentation in absence of selective pressure is measured by direct and indirect methods that confirm presence or absence of the recombinant DNA in production culture cells. Culture volume influence can be simulated by elongated culturing times meaning increased generation times. The more generations in fermentation, the more it is likely that a recombinant element is lost. Loss of a recombinant element is considered instability. Indirect methods rely on the association of selection cassettes with recombinant DNA, e.g. the antibiotic resistance cassettes in a plasmid. Production culture cells are plated on selective media, e.g. LB plates supplemented with antibiotics or other chemicals related to a selection system, and resistant colonies are considered as positive for the recombinant DNA associated to the respective selection chemical. In the case of a multiple plasmid system, resistant colonies to multiple antibiotics are counted and the proportion of cells containing all three resistances is considered the stable population. Alternatively, quantitative PCR can be used to measure the amount of recombinant DNA of the three recombinant elements in the presence, absence of selection, and at different time points of fermentation. Thus, the relative and absolute amount of recombinant DNA is measured and compared. Reproducibility of the production process is measured by the complete analysis of consistency batches by the methods stated in this application.

### Immunogenic Compositions

The modified pneumolysin proteins and conjugates (e.g. bioconjugate), of the invention are particularly suited for inclusion in immunogenic compositions and vaccines. The present invention provides an immunogenic composition comprising the modified pneumolysin protein of the invention, or the conjugate of the invention, or the bioconjugate of the invention.

Also provided is a method of making the immunogenic composition of the invention comprising the step of mixing the modified pneumolysin protein or the conjugate (e.g. bioconjugate) of the invention with a pharmaceutically acceptable excipient or carrier.

Immunogenic compositions comprise an immunologically effective amount of the modified pneumolysin protein or conjugate (e.g. bioconjugate) of the invention, as well as any other components. By “immunologically effective amount”, it is meant that the administration of that amount to an individual, either as a single dose or as part of a series is effective for treatment or prevention. This amount varies depending on the health and physical condition of the individual to be treated, age, the degree of protection desired, the formulation of the vaccine and other relevant factors. It is expected that the amount will fall in a relatively broad range that can be determined through routine trials.

Immunogenic compositions if the invention may also contain diluents such as water, saline, glycerol etc. Additionally, auxiliary substances, such as wetting or emulsifying agents, pH buffering substances, polyols and the like may be present.

The immunogenic compositions comprising the modified pneumolysin protein of the invention or conjugates (or bioconjugates) may comprise any additional components suitable for use in pharmaceutical administration. In specific embodiments, the immunogenic compositions of the invention are monovalent formulations. In other embodiments, the immunogenic compositions of the invention are multivalent formulations, e.g. bivalent, trivalent, and tetravalent formulations. For example, a multivalent formulation comprises more than one antigen for example more than one conjugate.

The immunogenic composition of the invention optionally further comprise additional antigens. Examples of such additional antigens are S. pneumoniae antigens selected from the following categories, such as proteins having a Type II Signal sequence motif of LXXC (where X is any amino acid, e.g. the polyhistidine triad family (PhtX)), choline binding proteins (e.g. CbpX (choline binding protein family), PcpA (pneumococcal choline-binding protein A)), proteins having a Type I Signal sequence motif (e.g. Sp101), and proteins having a LPXTG motif (where X is any amino acid, e.g. Sp128, Sp130). Thus, the immunogenic composition of the invention may comprise one or more S. pneumoniae proteins selected from polyhistidine triad family (PhtX), Choline Binding Protein family (CbpX), CbpX truncates, pneumococcal autolysin family (LytX) (e.g. LytA (N-acetylmuramoyl-l-alanine amidase), LytB, LytC), LytX truncates, CbpX truncate-LytX truncate chimeric proteins, PspA (pneumococcal surface protein A), PsaA (pneumococcal surface adhesion A), Sp128, Sp101, Sp130, Sp125 and Sp133. In a further embodiment, the immunogenic composition of the invention comprises 2 or more proteins selected from the group consisting of the polyhistidine triad family (PhtX), Choline Binding Protein family (CbpX), CbpX truncates, LytX family, LytX truncates, CbpX truncate-LytX truncate chimeric proteins (or fusions), PspA (pneumococcal surface protein A), PsaA (pneumococcal surface adhesion A), and Sp128. In a further embodiment, the immunogenic composition comprises 2 or more proteins selected from the group consisting of the polyhistidine triad family (PhtX) e.g. PhtD, Choline Binding Protein family (CbpX), CbpX truncates, LytX family, LytX truncates, CbpX truncate-LytX truncate chimeric proteins (or fusions), and Sp128.

In an embodiment, the S. pneumoniae antigen selected from member(s) of the polyhistidine triad family is PhtD. The term “PhtD” as used herein includes the full length protein with the signal sequence attached or the mature full length protein with the signal peptide (for example 20 amino acids at N-terminus) removed, and immunogenic fragments, variants and/or fusion proteins thereof, e.g. SEQ ID NO. 4 of WO00/37105. In one aspect, PhtD is the full length protein with the signal sequence attached e.g. SEQ ID NO. 4 of WO00/37105. In another aspect, PhtD is a sequence comprising the mature full length protein with the signal peptide (for example 20 amino acids at N-terminus) removed, e.g. amino acids 21-838 of SEQ ID NO. 4 of WO00/37105. Suitably, the PhtD sequence comprises an N-terminal methionine. The present invention also includes PhtD polypeptides which are immunogenic fragments of PhtD, variants of PhtD and/or fusion proteins of PhtD. For example, as described in WO00/37105, WO00/39299, U.S. Pat. No. 6,699,703 and WO09/12588.

### Vaccines

The present invention also provides a vaccine comprising an immunogenic composition of the invention and a pharmaceutically acceptable excipient or carrier.

Pharmaceutically acceptable excipients and carriers can be selected by those of skill in the art. For example, the pharmaceutically acceptable excipient or carrier can include a buffer, such as Tris (trimethamine), phosphate (e.g. sodium phosphate), acetate, borate (e.g. sodium borate), citrate, glycine, histidine and succinate (e.g. sodium succinate), suitably sodium chloride, histidine, sodium phosphate or sodium succinate. The pharmaceutically acceptable excipient may include a salt, for example sodium chloride, potassium chloride or magnesium chloride. Optionally, the pharmaceutically acceptable excipient contains at least one component that stabilizes solubility and/or stability. Examples of solubilizing/stabilizing agents include detergents, for example, laurel sarcosine and/or polysorbate (e.g. TWEEN™ 80). Examples of stabilizing agents also include poloxamer (e.g. poloxamer 124, poloxamer 188, poloxamer 237, poloxamer 338 and poloxamer 407). The pharmaceutically acceptable excipient may include a non-ionic surfactant, for example polyoxyethylene sorbitan fatty acid esters, Polysorbate-80 (TWEEN™ 80), Polysorbate-60 (TWEEN™ 60), Polysorbate-40 (TWEEN™ 40) and Polysorbate-20 (TWEEN™ 20), or polyoxyethylene alkyl ethers (suitably polysorbate-80). Alternative solubilizing/stabilizing agents include arginine, and glass forming polyols (such as sucrose, trehalose and the like). The pharmaceutically excipient may be a preservative, for example phenol, 2-phenoxyethanol, or thiomersal. Other pharmaceutically acceptable excipients include sugars (e.g. lactose, sucrose), and proteins (e.g. gelatine and albumin). Pharmaceutically acceptable carriers include water, saline solutions, aqueous dextrose and glycerol solutions. Numerous pharmaceutically acceptable excipients and carriers are described, for example, in Remington's Pharmaceutical Sciences, by E. W. Martin, Mack Publishing Co. Easton, Pa., 5th Edition (975).

In an embodiment, the immunogenic composition or vaccine of the invention additionally comprises one or more buffers, e.g. phosphate buffer and/or sucrose phosphate glutamate buffer. In other embodiments, the immunogenic composition or vaccine of the invention does not comprise a buffer.

In an embodiment, the immunogenic composition or vaccine of the invention additionally comprises one or more salts, e.g. sodium chloride, calcium chloride, sodium phosphate, monosodium glutamate, and aluminum salts (e.g. aluminum hydroxide, aluminum phosphate, alum (potassium aluminum sulfate), or a mixture of such aluminum salts). In other embodiments, the immunogenic composition or vaccine of the invention does not comprise a salt.

The immunogenic composition or vaccine of the invention may additionally comprise a preservative, e.g. a mercury derivative thimerosal. In a specific embodiment, the immunogenic composition or vaccine of the invention comprises 0.001% to 0.01% thimerosal. In other embodiments, the immunogenic composition or vaccine of the invention do not comprise a preservative.

The vaccine or immunogenic composition of the invention may also comprise an antimicrobial, typically when package in multiple dose format. For example, the immunogenic composition or vaccine of the invention may comprise 2-phenoxyethanol.

The vaccine or immunogenic composition of the invention may also comprise a detergent e.g. polysorbate, such as TWEEN™ 80. Detergents are generally present at low levels e.g. <0.01%, but higher levels have been suggested for stabilising antigen formulations e.g. up to 10%.

The immunogenic compositions of the invention can be included in a container, pack, or dispenser together with instructions for administration.

The immunogenic compositions or vaccines of the invention can be stored before use, e.g. the compositions can be stored frozen (e.g. at about −20° C. or at about −70° C.); stored in refrigerated conditions (e.g. at about 4° C.); or stored at room temperature.

The immunogenic compositions or vaccines of the invention may be stored in solution or lyophilized. In an embodiment, the solution is lyophilized in the presence of a sugar such as sucrose, trehalose or lactose. In another embodiment, the vaccines of the invention are lyophilized and extemporaneously reconstituted prior to use.

Vaccine preparation is generally described in Vaccine Design (“The subunit and adjuvant approach” (eds Powell M. F. & Newman M. J.) (1995) Plenum Press New York). Encapsulation within liposomes is described by Fullerton, U.S. Pat. No. 4,235,877.

### Adjuvants

In an embodiment, the immunogenic compositions or vaccines of the invention comprise, or are administered in combination with, an adjuvant. The adjuvant for administration in combination with an immunogenic composition or vaccine of the invention may be administered before, concomitantly with, or after administration of said immunogenic composition or vaccine. In some embodiments, the term “adjuvant” refers to a compound that when administered in conjunction with or as part of an immunogenic composition of vaccine of the invention augments, enhances and/or boosts the immune response to a bioconjugate, but when the compound is administered alone does not generate an immune response to the modified pneumolysin protein/conjugate/bioconjugate. In some embodiments, the adjuvant generates an immune response to the modified pneumolysin protein, conjugate or bioconjugate and does not produce an allergy or other adverse reaction.

In an embodiment, the immunogenic composition or vaccine of the invention is adjuvanted. Adjuvants can enhance an immune response by several mechanisms including, e.g. lymphocyte recruitment, stimulation of B and/or T cells, and stimulation of macrophages. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see United Kingdom Patent GB2220211), MF59 (Novartis), AS03 (GlaxoSmithKline), AS04 (GlaxoSmithKline), polysorbate 80 (TWEEN™ 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al. in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al. N. Engl. J. Med. 336, 86-91 (1997)). Another adjuvant is CpG (Bioworld Today, Nov. 15, 1998).

In one aspect of the invention, the adjuvant is an aluminum salt such as aluminum hydroxide gel (alum) or aluminium phosphate.

In another aspect of the invention, the adjuvant is selected to be a preferential inducer of either a TH1 or a TH2 type of response. High levels of Th1-type cytokines tend to favor the induction of cell mediated immune responses to a given antigen, whilst high levels of Th2-type cytokines tend to favour the induction of humoral immune responses to the antigen. It is important to remember that the distinction of Th1 and Th2-type immune response is not absolute. In reality an individual will support an immune response which is described as being predominantly Th1 or predominantly Th2. However, it is often convenient to consider the families of cytokines in terms of that described in murine CD4+ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p 145-173). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of 11-4, IL-5, IL-6, IL-10. Suitable adjuvant systems which promote a predominantly Th1 response include: Monophosphoryl lipid A or a derivative thereof, particularly 3-de-O-acylated monophosphoryl lipid A (3D-MPL) (for its preparation see GB 2220211 A); and a combination of monophosphoryl lipid A, for example 3-de-O-acylated monophosphoryl lipid A, together with either an aluminium salt (for instance aluminium phosphate or aluminium hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen [Thoelen et al. Vaccine (1998) 16:708-14; EP 689454-B1]. Unmethylated CpG containing oligonucleotides (WO 96/02555) are also preferential inducers of a TH1 response and are suitable for use in the present invention.

The vaccine or immunogenic composition of the invention may contain an oil in water emulsion, since these have been suggested to be useful as adjuvant compositions (EP 399843; WO 95/17210). Oil in water emulsions such as those described in WO95/17210 (which discloses oil in water emulsions comprising from 2 to 10% squalene, from 2 to 10% alpha tocopherol and from 0.3 to 3% tween 80 and their use alone or in combination with QS21 and/or 3D-MPL), WO99/12565 (which discloses oil in water emulsion compositions comprising a metabolisable oil, a saponin and a sterol and MPL) or WO99/11241 may be used. Further oil in water emulsions such as those disclosed in WO 09/127676 and WO 09/127677 are also suitable. A particularly potent adjuvant formulation involving QS21, 3D-MPL and tocopherol in an oil in water emulsion is described in WO 95/17210. In a specific embodiment, the immunogenic composition or vaccine additionally comprises a saponin, for example QS21. The immunogenic composition or vaccine may also comprise an oil in water emulsion and tocopherol (WO 95/17210).

### Method of Administration

Immunogenic compositions or vaccines of the invention may be used to protect or treat a mammal susceptible to infection, by means of administering said immunogenic composition or vaccine via systemic or mucosal route. These administrations may include injection via the intramuscular (IM), intraperitoneal, intradermal (ID) or subcutaneous routes; or via mucosal administration to the oral/alimentary, respiratory, genitourinary tracts. For example, intranasal (IN) administration may be used for the treatment of pneumonia or otitis media (as nasopharyngeal carriage of pneumococci can be more effectively prevented, thus attenuating infection at its earliest stage). Although the immunogenic composition or vaccine of the invention may be administered as a single dose, components thereof may also be co-administered together at the same time or at different times (for instance pneumococcal polysaccharides could be administered separately, at the same time or 1-2 weeks after the administration of any bacterial protein component of the vaccine for optimal coordination of the immune responses with respect to each other). For co-administration, the optional Th1 adjuvant may be present in any or all of the different administrations, however in one particular aspect of the invention it is present in combination with the modified pneumolysin protein component of the immunogenic composition or vaccine. In addition to a single route of administration, 2 different routes of administration may be used. For example, polysaccharides may be administered IM (or ID) and bacterial proteins may be administered IN (or ID). In addition, the vaccines of the invention may be administered IM for priming doses and IN for booster doses.

In one aspect, the immunogenic composition or vaccine of the invention is administered by the intramuscular delivery route. Intramuscular administration may be to the thigh or the upper arm. Injection is typically via a needle (e.g. a hypodermic needle), but needle-free injection may alternatively be used. A typical intramuscular dose is 0.5 ml.

In another aspect, the immunogenic composition or vaccine of the invention is administered by the intradermal administration. Human skin comprises an outer “horny” cuticle, called the stratum corneum, which overlays the epidermis. Underneath this epidermis is a layer called the dermis, which in turn overlays the subcutaneous tissue. The conventional technique of intradermal injection, the “mantoux procedure”, comprises steps of cleaning the skin, and then stretching with one hand, and with the bevel of a narrow gauge needle (26 to 31 gauge) facing upwards the needle is inserted at an angle of between 10 to 15°. Once the bevel of the needle is inserted, the barrel of the needle is lowered and further advanced whilst providing a slight pressure to elevate it under the skin. The liquid is then injected very slowly thereby forming a bleb or bump on the skin surface, followed by slow withdrawal of the needle.

More recently, devices that are specifically designed to administer liquid agents into or across the skin have been described, for example the devices described in WO 99/34850 and EP 1092444, also the jet injection devices described for example in WO 01/13977; U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO 97/37705 and WO 97/13537. Alternative methods of intradermal administration of the vaccine preparations may include conventional syringes and needles, or devices designed for ballistic delivery of solid vaccines (WO 99/27961), or transdermal patches (WO 97/48440; WO 98/28037); or applied to the surface of the skin (transdermal or transcutaneous delivery WO 98/20734; WO 98/28037).

In another aspect, the immunogenic composition or vaccine of the invention is administered by the intranasal administration. Typically, the immunogenic composition or vaccine is administered locally to the nasopharyngeal area, e.g. without being inhaled into the lungs. It is desirable to use an intranasal delivery device which delivers the immunogenic composition or vaccine formulation to the nasopharyngeal area, without or substantially without it entering the lungs. Suitable devices for intranasal administration of the vaccines according to the invention are spray devices. Suitable commercially available nasal spray devices include ACCUSPRAY™ (Becton Dickinson).

In an embodiment, spray devices for intranasal use are devices for which the performance of the device is not dependent upon the pressure applied by the user. These devices are known as pressure threshold devices. Liquid is released from the nozzle only when a threshold pressure is applied. These devices make it easier to achieve a spray with a regular droplet size. Pressure threshold devices suitable for use with the present invention are known in the art and are described for example in WO91/13281 and EP311 863 and EP516636, incorporated herein by reference. Such devices are commercially available from Pfeiffer GmbH and are also described in Bommer, R. Pharmaceutical Technology Europe, Sept 1999.

In another embodiment, intranasal devices produce droplets (measured using water as the liquid) in the range 1 to 200 μm, e.g. 10 to 120 μm. Below 10 μm there is a risk of inhalation, therefore it is desirable to have no more than about 5% of droplets below 10 μm. Droplets above 120 μm do not spread as well as smaller droplets, so it is desirable to have no more than about 5% of droplets exceeding 120 μm.

Following an initial vaccination, subjects may receive one or several booster immunizations adequately spaced.

The immunogenic composition or vaccine of the present invention may be used to protect or treat a mammal, e.g. human, susceptible to infection, by means of administering said immunogenic composition or vaccine via a systemic or mucosal route. These administrations may include injection via the intramuscular (IM), intraperitoneal (IP), intradermal (ID) or subcutaneous (SC) routes; or via mucosal administration to the oral/alimentary, respiratory, genitourinary tracts. Although the vaccine of the invention may be administered as a single dose, components thereof may also be co-administered together at the same time or at different times (for instance pneumococcal saccharide conjugates could be administered separately, at the same time or 1-2 weeks after the administration of the any modified pneumolysin protein, conjugate or bioconjugate of the invention for optimal coordination of the immune responses with respect to each other). For co-administration, the optional adjuvant may be present in any or all of the different administrations. In addition to a single route of administration, 2 different routes of administration may be used. For example, polysaccharide conjugates may be administered IM (or ID) and the modified pneumolysin protein, conjugate or bioconjugate of the invention may be administered IN (or ID). In addition, the immunogenic compositions or vaccines of the invention may be administered IM for priming doses and IN for booster doses.

### Dosage

The amount of conjugate antigen in each immunogenic composition or vaccine dose is selected as an amount which induces an immunoprotective response without significant, adverse side effects in typical vaccines. Such amount will vary depending upon which specific immunogen is employed and how it is presented. The content of modified pneumolysin protein will typically be in the range 1-100 μg, suitably 5-50 μg. The content of saccharide will typically be in the range 0.1-10 μg, suitably 1-5 μg.

A dose which is in a volume suitable for human use is generally between 0.25 and 1.5 ml, although, for administration to the skin a lower volume of between 0.05 ml and 0.2 ml may be used. In one embodiment, a human dose is 0.5 ml. In a further embodiment, a human dose is higher than 0.5 ml, for example 0.6, 0.7, 0.8, 0.9 or 1 ml. In a further embodiment, a human dose is between 1 ml and 1.5 ml. In another embodiment, in particular when the immunogenic composition is for the paediatric population, a human dose may be less than 0.5 ml such as between 0.25 and 0.5 ml.

### Prophylactic and Therapeutic Uses

The present invention also provides methods of treating and/or preventing bacterial infections of a subject comprising administering to the subject a modified pneumolysin protein, conjugate or bioconjugate of the invention. The modified pneumolysin protein, conjugate or bioconjugate may be in the form of an immunogenic composition or vaccine. In a specific embodiment, the immunogenic composition or vaccine of the invention is used in the prevention of infection of a subject (e.g. human subjects) by a bacterium. Bacteria infections that can be treated and/or prevented using the modified pneumolysin protein, conjugate or bioconjugate of the invention include those caused by Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species. In a specific embodiment, the immunogenic composition or vaccine of the invention is used to treat or prevent an infection by Streptococcus species (e.g. Streptococcus pneumoniae).

Also provided herein are methods of inducing an immune response in a subject against a bacterium, comprising administering to the subject a modified pneumolysin protein, or conjugate or bioconjugate of the invention (or immunogenic composition or vaccine). In one embodiment, said subject has bacterial infection at the time of administration. In another embodiment, said subject does not have a bacterial infection at the time of administration. The modified pneumolysin protein, conjugate or bioconjugate of the invention can be used to induce an immune response against Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species. In a specific embodiment, modified pneumolysin protein, or conjugate or bioconjugate of the invention is used to induce an immune response against Streptococcus species (e.g. Streptococcus pneumoniae).

Also provided herein are methods of inducing the production of opsonophagocytic antibodies in a subject against a bacterium, comprising administering to the subject a modified pneumolysin protein, or conjugate or bioconjugate of the invention (or immunogenic composition or vaccine). In one embodiment, said subject has bacterial infection at the time of administration. In another embodiment, said subject does not have a bacterial infection at the time of administration. The modified pneumolysin protein, or conjugate or bioconjugate of the invention (or immunogenic composition or vaccine) provided herein can be used to induce the production of opsonophagocytic antibodies against Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species. In a specific embodiment, a modified pneumolysin protein, or conjugate or bioconjugate of the invention (or immunogenic composition or vaccine) is used to induce the production of opsonophagocytic antibodies against Streptococcus species (e.g. Streptococcus pneumoniae).

In an embodiment, the present invention is an improved method to elicit an immune response in infants (defined as 0-2 years old in the context of the present invention) by administering a therapeutically effective amount of an immunogenic composition or vaccine of the invention (a paediatric vaccine). In an embodiment, the vaccine is a paediatric vaccine.

In an embodiment, the present invention is an improved method to elicit an immune response in the elderly population (in the context of the present invention a patient is considered elderly if they are 50 years or over in age, typically over 55 years and more generally over 60 years) by administering a therapeutically effective amount of the immunogenic composition or vaccine of the invention. In an embodiment, the vaccine is a vaccine for the elderly.

The present invention provides a method for the treatment or prevention of Streptococcus pneumoniae infection in a subject in need thereof comprising administering to said subject a therapeutically effective amount of the modified pneumolysin protein of the invention, or the conjugate of the invention, or the bioconjugate of the invention, or the immunogenic composition or vaccine of the invention.

The present invention provides a method of immunising a human host against Streptococcus pneumoniae infection comprising administering to the host an immunoprotective dose of the modified pneumolysin protein of the invention, or the conjugate of the invention, or the bioconjugate of the invention, or the immunogenic composition or vaccine of the invention.

The present invention provides a method of inducing an immune response to Streptococcus pneumoniae in a subject, the method comprising administering a therapeutically or prophylactically effective amount of the modified pneumolysin protein of the invention, or the conjugate of the invention, or the bioconjugate of the invention, or the immunogenic composition or vaccine of the invention.

The present invention provides a modified pneumolysin protein of the invention, or the conjugate of the invention, or the bioconjugate of the invention, or the immunogenic composition or vaccine of the invention for use in the treatment or prevention of a disease caused by S. pneumoniae infection.

The present invention provides use of the modified pneumolysin protein of the invention, or the conjugate of the invention, or the bioconjugate of the invention in the manufacture of a medicament for the treatment or prevention of a disease caused by Streptococcus pneumoniae infection.

The disease caused by Streptococcus pneumoniae infection may be selected from pneumonia, invasive pneumococcal disease (IPD), exacerbations of chronic obstructive pulmonary disease (eCOPD), otitis media, meningitis, bacteraemia, pneumonia and/or conjunctivitis. Where the human host is an infant (defined as 0-2 years old in the context of the present invention), the disease may be selected from otitis media, meningitis, bacteraemia, pneumonia and/or conjunctivitis. In one aspect, where the human host is an infant (defined as 0-2 years old in the context of the present invention), the disease is selected from otitis media and/or pneumonia. Where the human host is elderly (i.e. 50 years or over in age, typically over 55 years and more generally over 60 years), the disease may be selected from pneumonia, invasive pneumococcal disease (IPD), and/or exacerbations of chronic obstructive pulmonary disease (eCOPD). In one aspect, where the human host is elderly, the disease is invasive pneumococcal disease (IPD). In another aspect, where the human host is elderly, the disease is exacerbations of chronic obstructive pulmonary disease (eCOPD).

All references or patent applications cited within this patent specification are incorporated by reference herein.

In order that this invention may be better understood, the following examples are set forth. These examples are for purposes of illustration only, and are not to be construed as limiting the scope of the invention in any manner.

Aspects of the invention are summarised in the subsequence numbered paragraphs:
1. A modified pneumolysin protein having an amino acid sequence of SEQ ID NO. 1 or an amino acid sequence at least 80%, 85%, 90%, 92%, 95%, 96%, 97%, 98% or 99% identical to SEQ ID NO. 1 (e.g. SEQ ID NO. 88), modified in that the amino acid sequence comprises one or more consensus sequence(s) selected from: D/E-X-N-Z-S/T (SEQ ID NO. 28) and K-D/E-X-N-Z-S/T-K (SEQ ID NO. 30), wherein X and Z are independently any amino acid apart from proline.
2. The modified pneumolysin protein of paragraph 1, wherein one or more amino acids (e.g. 1-7 amino acids, e.g. one amino acid) of the amino acid sequence of SEQ ID NO. 1 or an amino acid sequence at least 80%, 85%, 90%, 92%, 95%, 96%, 97%, 98% or 99% identical to SEQ ID NO. 1 (e.g. SEQ ID NO. 88) have been substituted by a D/E-X-N-Z-S/T (SEQ ID NO. 28) or K-D/E-X-N-Z-S/T-K (SEQ ID NO. 30) consensus sequence.
3. The modified pneumolysin protein of paragraph 1 or paragraph 2, wherein a consensus sequence selected from D/E-X-N-Z-S/T (SEQ ID NO. 28) and K-D/E-X-N-Z-S/T-K (SEQ ID NO. 30) is located at a position within the long N terminal surface loop or the short C terminal loop of SEQ ID NO. 1 or an equivalent position within an amino acid sequence at least 80%, 85%, 90%, 92%, 95%, 96%, 97%, 98% or 99% identical to SEQ ID NO. 1 (e.g. SEQ ID NO. 88).
4. The modified pneumolysin protein of any one of paragraphs 1-3, wherein a consensus sequence selected from D/E-X-N-Z-S/T (SEQ ID NO. 28) and K-D/E-X-N-Z-S/T-K (SEQ ID NO. 30) has been added at, or substituted for, one or more amino acids, between amino acid residues 22-57 (e.g. between amino acid residues 24-29, or amino acid residues 24, 27 or 29) of SEQ ID NO. 1 or at an equivalent position within an amino acid sequence at least 80%, 85%, 90%, 92%, 95%, 96%, 97%, 98% or 99% identical to SEQ ID NO. 1 (e.g. SEQ ID NO. 88).
5. The modified pneumolysin protein of any one of paragraphs 1-3, wherein a consensus sequence selected from D/E-X-N-Z-S/T (SEQ ID NO. 28) and K-D/E-X-N-Z-S/T-K (SEQ ID NO. 30) has been added at, or substituted, for one or more amino acids, between amino acid residues 360-470 (e.g. between amino acid residues 427-437, between amino acids 431-434, or amino acid residues 431 or 434) of SEQ ID NO. 1 or at an equivalent position within an amino acid sequence at least 80%, 85%, 90%, 92%, 95%, 96%, 97%, 98% or 99% identical to SEQ ID NO. 1 (e.g. SEQ ID NO. 88).
6. The modified pneumolysin protein of any one of paragraphs 1-5, comprising a single consensus sequence selected from D/E-X-N-Z-S/T (SEQ ID NO. 28) and K-D/E-X-N-Z-S/T-K (SEQ ID NO. 30).
7. The modified pneumolysin protein of paragraph 6, wherein the consensus sequence K-D/E-X-N-Z-S/T-K (SEQ ID NO. 30) has been substituted for amino acid residue 434 in SEQ ID NO. 1, or substituted in an amino acid sequence at least 80%, 85%, 90%, 92%, 95%, 96%, 97%, 98% or 99% identical to SEQ ID NO. 1 at an amino acid position equivalent to amino acid residue 434 in SEQ ID NO. 1 (e.g. SEQ ID NO. 88).
8. The modified pneumolysin protein of any one of paragraphs 1-7, wherein X is Q (glutamine) and Z is A (alanine) (e.g. K-D-Q-N-A-T-K (SEQ ID NO. 31)).
9. A modified pneumolysin protein comprising (or consisting of) an amino acid sequence which is at least 80%, 85%, 90%, 92%, 95%, 96%, 97%, 98%, 99% or 100% identical to the sequence of SEQ ID NO. 2, said amino acid sequence comprising a D/E-X-N-Z-S/T (SEQ ID NO. 28) or K-D/E-X-N-Z-S/T-K (SEQ ID NO. 30) consensus sequence, wherein X and Z are independently any amino acid apart from proline.
10. The modified pneumolysin protein of any one of paragraphs 1-9, wherein the amino acid sequence is further modified by at least one amino acid substitution selected from G₂₉₃ to C, T₆₅ to C, C₄₂₈ to A, A₃₇₀ to E, W₄₃₃ to E and L₄₆₀ to E with reference to the amino acid sequence of SEQ ID NO. 1 (or an equivalent position in an amino acid sequence at least 80%, 85%, 90%, 92%, 95%, 96%, 97%, 98% or 99% identical to SEQ ID NO. 1, e.g. SEQ ID NO. 88).
11. A modified pneumolysin protein comprising (or consisting of) an amino acid sequence which is at least 80%, 85%, 90%, 92%, 95%, 97%, 98%, 99% or 100% identical to a sequence selected from SEQ ID NOs: 3-10, said amino acid sequence comprising a D/E-X-N-Z-S/T (SEQ ID NO. 28) or K-D/E-X-N-Z-S/T-K (SEQ ID NO. 30) consensus sequence wherein X and Z are independently any amino acid apart from proline and at least one amino acid substitution selected from G₂₉₃ to C, T₆₅ to C, C₄₂₈ to A, A₃₇₀ to E, W₄₃₃ to E and L₄₆₀ to E.
12. The modified pneumolysin protein of any one of paragraphs 1-11, wherein the amino acid sequence further comprises a peptide tag which is useful for the purification of the pneumolysin protein, optionally said peptide tag comprising six histidine residues and optionally said peptide tag located at the C-terminus of the amino acid sequence, optionally said modified pneumolysin protein having an amino acid sequence at least 97%, 98%, 99% or 100% identical to SEQ ID NO. 11 or SEQ ID NO. 12.
13. The modified pneumolysin protein of any one of paragraphs 1-12, wherein the amino acid sequence further comprises a signal sequence which is capable of directing the pneumolysin protein to the periplasm of a host cell (e.g. bacterium), optionally said signal sequence being selected from SEQ ID NO. 13-20, optionally said modified pneumolysin protein having an amino acid sequence at least 97%, 98%, 99% or 100% identical to SEQ ID NO. 21 or SEQ ID NO. 22.
14. The modified pneumolysin protein of any one of paragraphs 1-13, wherein the modified pneumolysin protein is glycosylated.
15. A conjugate (e.g. bioconjugate) comprising a modified pneumolysin protein of paragraphs 1-12 and 14, wherein the modified pneumolysin protein is linked to an antigen.
16. The conjugate according to paragraph 15, wherein the modified pneumolysin protein is covalently linked to an antigen through a chemical linkage obtainable using a chemical conjugation method, optionally selected from the group consisting of carbodiimide chemistry, reductive animation, cyanylation chemistry (for example CDAP chemistry), maleimide chemistry, hydrazide chemistry, ester chemistry, and N-hydroysuccinimide chemistry either directly or via a linker.
17. The conjugate (e.g. bioconjugate) of paragraph 15 or paragraph 16, wherein the antigen is linked to an amino acid on the modified pneumolysin protein selected from asparagine, aspartic acid, glutamic acid, lysine, cysteine, tyrosine, histidine, arginine or tryptophan (e.g. asparagine).
18. The conjugate (e.g. bioconjugate) of any one of paragraphs 15-17, wherein the antigen is a saccharide, optionally a bacterial capsular saccharide (e.g. from Streptococcus pneumoniae) optionally selected from a Streptococcus pneumoniae serotype 1, 2, 3, 4, 5, 6A, 6B, 7A, 7B, 7C, 8, 9A, 9L, 9N, 9V, 10A, 10B, 10C, 10F, 11A, 11B, 11C, 11D, 11F, 12A, 12B, 12F, 13, 14, 15A, 15B, 15C, 15F, 16A, 16F, 17A, 17F, 18A, 18B, 18C, 18F, 19A, 19B, 19C, 19F, 20, 21, 22A, 22F, 23A, 23B, 23F, 24A, 24B, 24F, 25A, 25F, 26, 27, 28A, 28F, 29, 31, 32A, 32F, 33A, 33B, 33C, 33D, 33F, 34, 35A, 35B, 35C, 35D, 35F, 36, 37, 38, 39, 40, 41A, 41F, 42, 43, 44, 45, 46, 47A, 47F or 48 capsular saccharide.
19. The conjugate (e.g. bioconjugate) of paragraph 18, wherein the antigen is a bacterial capsular saccharide from Streptococcus pneumoniae selected from a Streptococcus pneumoniae serotype 4, or Streptococcus pneumoniae serotype 33F capsular saccharide.
20. The conjugate (e.g. bioconjugate) of paragraph 18, wherein the antigen is a hybrid oligosaccharide or polysaccharide having a structure:

(B)ₙ-A→
wherein A is an oligosaccharide repeat unit containing at least 2, 3, 4, 5, 6, 7 or 8 monosaccharides, with a hexose monosaccharide derivative at the reducing end (indicated by arrow);
wherein B is an oligosaccharide repeat unit containing at least 2, 3, 4, 5, 6, 7 or 8 monosaccharides;
wherein A and B are different oligosaccharide repeat units; and
wherein n is at least 2, 3, 4, 5, 6, 7, 8, 9, 10, 11, 12, 13, 14, 15, 16, 17, 18, 19, or at least 20, optionally wherein the hexose monosaccharide at the reducing end of the repeat is selected from the group consisting of glucose, galactose, rhamnose, arabinotol, fucose and mannose.
21. A polynucleotide encoding the modified pneumolysin protein of any one of paragraphs 1-14.
22. A vector comprising the polynucleotide of paragraph 21.
23. A host cell comprising:
   i) one or more nucleic acids that encode glycosyltransferase(s);
   ii) a nucleic acid that encodes an oligosaccharyl transferase;
   iii) a nucleic acid that encodes a modified pneumolysin protein according to any one of paragraphs 1-14; and optionally
   iv) a nucleic acid that encodes a polymerase (e.g. wzy).
24. The host cell of paragraph 23, wherein said host cell comprises (a) a glycosyltransferase that assembles a hexose monosaccharide derivative onto undecaprenyl pyrophosphate (Und-PP) and (b) one or more glycosyltransferases capable of adding a monosaccharide to the hexose monosaccharide derivative assembled on Und-PP.
25. The host cell of paragraph 24, wherein said glycosyltransferase that assembles a hexose monosaccharide derivative onto Und-PP is heterologous to the host cell and/or heterologous to one or more of the genes that encode glycosyltransferase(s) optionally wherein said glycosyltransferase that assembles a hexose monosaccharide derivative onto Und-PP is from Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species, optionally wecA (e.g. wecA from E. coli).
26. The host cell of any one of paragraphs 24-25, wherein said hexose monosaccharide derivative is any monosaccharide in which C-2 position is modified with an acetamido group such as N-acetylglucosamine (GlcNAc), N-acetylgalactoseamine (GalNAc), 2,4-Diacetamido-2,4,6-trideoxyhexose (DATDH). N-acetylfucoseamine (FucNAc), or N-acetylquinovosamine (QuiNAc).
27. The host cell of any one of paragraphs 24-26, wherein said one or more glycosyltransferases capable of adding a monosaccharide to the hexose monosaccharide derivative assembled on Und-PP is the galactofuranosyltransferase (wbeY) from E. coli O28 or the galactofuranosyltransferase (wfdK) from E. coli O167 or are the galactofuranosyltransferase (wbeY) from E. coli O28 and the galactofuranosyltransferase (wfdK) from E. coli O167.
28. The host cell of any one of paragraphs 23-27 wherein the host cell comprises glycosyltransferases sufficient for synthesis repeat units of the CP4 saccharide comprising wzg, wzh, wzd and/or wze from S. pneumoniae CP4 and optionally wciI, wciJ, wciK, wciL, wzy wciM, wzx, mnaA, fnlA, fnlB and fnlC from S. pneumoniae CP4.
29. The host cell of any one of paragraphs 23-27 wherein the host cell comprises glycosyltransferases sufficient for synthesis repeat units of the CP12F saccharide comprising wzg, wzh, wzd and/or wze from S. pneumoniae CP12F.
30. The host cell of any one of paragraphs 23-27, wherein the host cell comprises glycosyltransferases sufficient for synthesis of repeat units of the CP33F saccharide comprising wciC, wciD, wciE, and/or wciF from S. pneumoniae CP33F and optionally wchA and/or wciB from S. pneumoniae CP33F.
31. The host cell of paragraph 30, wherein said host cell is capable of producing a hybrid oligosaccharide or polysaccharide, wherein said hybrid oligosaccharide or polysaccharide is identical to S. pneumoniae CP33F, with the exception of the fact that said hybrid oligosaccharide or polysaccharide comprises a hexose monosaccharide derivative at the reducing end of the first repeat unit in place of the hexose monosaccharide normally present at the reducing end of the first repeat unit of S. pneumoniae CP33F.
32. The host cell of any one of paragraphs 24-31, wherein the glycosyltransferases comprise a glycosyltransferase that is capable of adding the hexose monosaccharide present at the reducing end of the first repeat unit of the donor oligosaccharide or polysaccharide to the hexose monosaccharide derivative, optionally wherein said one or more glycosyltransferases capable of adding a monosaccharide to the hexose monosaccharide derivative comprise galactosyltransferase (wclP), optionally from E. coli O21, and optionally comprising a glycosyltransferase that is capable of adding the monosaccharide that is adjacent to the hexose monosaccharide present at the reducing end of the first repeat unit of the donor oligosaccharide or polysaccharide to the hexose monosaccharide present at the reducing end of the first repeat unit of the donor oligosaccharide or polysaccharide, optionally glucosyltransferase (wclQ), optionally from E. coli O21.
33. The host cell of any one of paragraphs 23-32 wherein the oligosaccharyl transferase is derived from Campylobacter jejuni, optionally wherein said oligosaccharyl transferase is pglB of C. jejuni, optionally wherein the pglB gene of C. jejuni is integrated into the host cell genome and optionally wherein at least one gene of the host cell has been functionally inactivated or deleted, optionally wherein the waaL gene of the host cell has been functionally inactivated or deleted, optionally wherein the waaL gene of the host cell has been replaced by a nucleic acid encoding an oligosaccharyltransferase, optionally wherein the waaL gene of the host cell has been replaced by C. jejuni pglB.
34. The host cell of any one of paragraphs 23-33, wherein said host cell comprises a nucleic acid that encodes a capsular polysaccharide polymerase (e.g. wzy) or an O antigen polymerase (e.g. wzy), optionally said capsular polysaccharide polymerase is from Streptococcus pneumoniae, optionally from S. pneumoniae CP1, CP2, CP4, CP5, CP6 (A,B,C,D), CP7 (A,B, C), CP8, CP9 (A,L,N,V), CP10 (A,B,C,F), CP11 (A, B,C,D,F), CP12(A,B,F), CP13, CP14 CP15(A,B,C,F), CP16(A,F), CP17(A,F), CP18(A,B,C,F), CP19(A,B,C,F), CP20,CP21, CP22(A,F), CP23(A,B,F), CP24(A,B,F), CP25(A,F), CP26, CP27, CP28(A,F), CP29, CP31, CP32(A,F), CP33(A,B,C,D,F), CP34, CP35(A,B,C,D,F), CP36, CP38, CP39, CP40, CP41(A,F), CP42, CP43, CP44, CP45, CP46, CP47(A,F), or CP48.
35. The host cell of any one of paragraphs 23-34, wherein said host cell comprises a nucleic acid that encodes a flippase (wzx), optionally wherein said flippase is from Streptococcus pneumoniae, optionally from S. pneumoniae CP1, CP2, CP4, CP5, CP6(A,B,C,D), CP7(A,B,C), CP8, CP9(A,L,N,V), CP10(A,B,C,F), CP11(A,B,C,D,F), CP12(A,B,F), CP13, CP14 CP15(A,B,C,F), CP16(A,F), CP17(A,F), CP18(A,B,C,F), CP19(A,B,C,F), CP20, CP21, CP22(A,F), CP23(A,B,F), CP24(A,B,F), CP25(A,F), CP26, CP27, CP28(A,F), CP29, CP31, CP32(A,F), CP33(A,B,C,D,F), CP34, CP35(A,B,C,D,F), CP36, CP38, CP39, CP40, CP41(A,F), CP42, CP43, CP44, CP45, CP46, CP47(A,F), or CP48.
36. The host cell of any one of paragraphs 23-35, wherein said host cell further comprises an enzyme capable of modifying a monosaccharide, optionally an epimerase, optionally wherein said epimerase is from Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species, optionally wherein said epimerase is from E. coli, optionally Z3206 from E. coli O157 or galE.
37. The host cell of any one of paragraphs 23-36, wherein the nucleic acid that encodes the modified pneumolysin protein is in a plasmid in the host cell.
38. The host cell of any one of paragraphs 23-37, wherein the host cell is E. coli.
39. A method of producing a bioconjugate that comprises a modified pneumolysin protein linked to a saccharide, said method comprising (i) culturing the host cell of any one of paragraphs 23-38 under conditions suitable for the production of proteins and (ii) isolating the bioconjugate.
40. A bioconjugate produced by the process of paragraph 39, wherein said bioconjugate comprises a saccharide linked to a modified pneumolysin protein.
41. An immunogenic composition comprising the modified pneumolysin protein of any one of paragraphs 1-14, or the conjugate of any one of paragraphs 15-22, or the bioconjugate of 40.
42. A method of making the immunogenic composition of paragraph 41 comprising the step of mixing the modified pneumolysin protein or the conjugate or the bioconjugate with a pharmaceutically acceptable excipient or carrier.
43. A vaccine comprising the immunogenic composition of paragraph 41 and a pharmaceutically acceptable excipient or carrier.
44. A method for the treatment or prevention of Streptococcus pneumoniae infection in a subject in need thereof comprising administering to said subject a therapeutically effective amount of the modified pneumolysin protein of any one of paragraphs 1-14, or the conjugate of any one of paragraphs 15-22, or the bioconjugate of paragraph 40.
45. A method of immunising a human host against Streptococcus pneumoniae infection comprising administering to the host an immunoprotective dose of the modified pneumolysin protein of any one of paragraphs 1-14, or the conjugate of any one of paragraphs 15-22, or the bioconjugate of paragraph 40.
46. A method of inducing an immune response to Streptococcus pneumoniae in a subject, the method comprising administering a therapeutically or prophylactically effective amount of the modified pneumolysin protein of any one of paragraphs 1-14, or the conjugate of any one of paragraphs 15-22, or the bioconjugate of 40.
47. A modified pneumolysin protein of any one of paragraphs 1-14, or the conjugate of any one of paragraphs 15-22, or the bioconjugate of paragraph 40 for use in the treatment or prevention of a disease caused by S. pneumoniae infection.
48. Use of the modified pneumolysin protein of any one of paragraphs 1-14, or the conjugate of any one of paragraphs 15-22, or the bioconjugate of paragraph 40 in the manufacture of a medicament for the treatment or prevention of a disease caused by Streptococcus pneumoniae infection.

Sequences of proteins and nucleic acids

SEQ ID NO: 1 - pneumolysin sequence (with N-terminal serine)
SANKAVNDFIL AMNYDKKKLL THQGESIENR FIKEGNQLPD EFVVIERKKR SLSTNTSDIS
VTATNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LGGDPSSGAR
VVTGKVDMVE DLIQEGSRFT ADHPGLPISY TTSFLRDNVV ATF QNSTDYV ETKVTAYRNG
DLLLDHSGAY VAQYYITWNE LSYDHQGKEV LTPKAWDRNG QDLTAHFTTS IPLKGNVRNL
SVKIRECTGL AW E W WRTVYEKTDL PLVRKRTISI WGTTLYPQVE DKVEND
SEQ ID NO: 2 - modified pneumolysin with glycosite
ANKAVNDFIL AMNYDKKKLL THQGESIENR FIKEGNQLPD EFVVIERKKR SLSTNTSDIS
VTATNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LGGDPSSGAR
VVTGKVDMVE DLIQEGSRFT ADHPGLPISY TTSFLRDNVV ATF QNSTDYV ETKVTAYRNG
DLLLDHSGAY VAQYYITWNE LSYDHQGKEV LTPKAWDRNG QDLTAHFTTS IPLKGNVRNL
SVKIRECTGL AW KDQNATK W WRTVYEKTDL PLVRKRTISI WGTTLYPQVE DKVEND
SEQ ID NO: 3 - modified pneumolysin with glycosite and further
modification
ANKAVNDFIL AMNYDKKKLL THQGESIENR FIKEGNQLPD EFVVIERKKR SLSTNTSDIS
VTACNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LGGDPSSGAR
VVTGKVDMVE DLIQEGSRFT ADHPGLPISY TTSFLRDNVV ATF QNSTDYV ETKVTAYRNG
DLLLDHSGAY VAQYYITWNE LSYDHQGKEV LTPKAWDRNG QDLTAHFTTS IPLKGNVRNL
SVKIRECTGL AW KDQNATK W WRTVYEKTDL PLVRKRTISI WGTTLYPQVE DKVEND
SEQ ID NO: 4 - modified pneumolysin with glycosite and further
modification
ANKAVNDFIL AMNYDKKKLL THQGESIENR FIKEGNQLPD EFVVIERKKR SLSTNTSDIS
VTATNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LCGDPSSGAR
VVTGKVDMVE DLIQEGSRFT ADHPGLPISY TTSFLRDNVV ATF QNSTDYV ETKVTAYRNG
DLLLDHSGAY VAQYYITWNE LSYDHQGKEV LTPKAWDRNG QDLTAHFTTS IPLKGNVRNL
SVKIRECTGL AW KDQNATK W WRTVYEKTDL PLVRKRTISI WGTTLYPQVE DKVEND
SEQ ID NO: 5 - modified pneumolysin with glycosite and further
modification
ANKAVNDFIL AMNYDKKKLL THQGESIENR FIKEGNQLPD EFVVIERKKR SLSTNTSDIS
VTATNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LGGDPSSGAR
VVTGKVDMVE DLIQEGSRFT ADHPGLPISY TTSFLRDNVV ATF QNSTDYV ETKVTAYRNG
DLLLDHSGEY VAQYYITWNE LSYDHQGKEV LTPKAWDRNG QDLTAHFTTS IPLKGNVRNL
SVKIRECTGL AW KDQNATK W WRTVYEKTDL PLVRKRTISI WGTTLYPQVE DKVEND
SEQ ID NO: 6 - modified pneumolysin with glycosite and further
modification
ANKAVNDFIL AMNYDKKKLL THQGESIENR FIKEGNQLPD EFVVIERKKR SLSTNTSDIS
VTATNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LGGDPSSGAR
VVTGKVDMVE DLIQEGSRFT ADHPGLPISY TTSFLRDNVV ATF QNSTDYV ETKVTAYRNG
DLLLDHSGAY VAQYYITWNE LSYDHQGKEV LTPKAWDRNG QDLTAHFTTS IPLKGNVRNL
SVKIREATGL AW KDQNATK W WRTVYEKTDL PLVRKRTISI WGTTLYPQVE DKVEND
SEQ ID NO: 7 - modified pneumolysin with glycosite and further
modification
ANKAVNDFIL AMNYDKKKLL THQGESIENR FIKEGNQLPD EFVVIERKKR SLSTNTSDIS
VTATNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LGGDPSSGAR
VVTGKVDMVE DLIQEGSRFT ADHPGLPISY TTSFLRDNVV ATF QNSTDYV ETKVTAYRNG
DLLLDHSGAY VAQYYITWNE LSYDHQGKEV LTPKAWDRNG QDLTAHFTTS IPLKGNVRNL
SVKIRECTGL AE KDQNATK W WRTVYEKTDL PLVRKRTISI WGTTLYPQVE DKVEND
SEQ ID NO: 8 - modified pneumolysin with glycosite and further
modification
ANKAVNDFIL AMNYDKKKLL THQGESIENR FIKEGNQLPD EFVVIERKKR SLSTNTSDIS
VTATNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LGGDPSSGAR
VVTGKVDMVE DLIQEGSRFT ADHPGLPISY TTSFLRDNVV ATF QNSTDYV ETKVTAYRNG
DLLLDHSGAY VAQYYITWNE LSYDHQGKEV LTPKAWDRNG QDLTAHFTTS IPLKGNVRNL
SVKIRECTGL AW KDQNATK W WRTVYEKTDL PLVRKRTISI WGTTEYPQVE DKVEND
SEQ ID NO: 9 - modified pneumolysin with glycosite and further
modification (with N-terminal serine)
SANKAVNDFIL AMNYDKKKLL THQGESIENR FIKEGNQLPD EFVVIERKKR SLSTNTSDIS
VTACNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LCGDPSSGAR
VVTGKVDMVE DLIQEGSRFT ADHPGLPISY TTSFLRDNVV ATF QNSTDYV ETKVTAYRNG
DLLLDHSGEY VAQYYITWNE LSYDHQGKEV LTPKAWDRNG QDLTAHFTTS IPLKGNVRNL
SVKIREATGL AE KDQNATK W WRTVYEKTDL PLVRKRTISI WGTTEYPQVE DKVEND
SEQ ID NO: 10 - modified pneumolysin with glycosite and further
modification (without N-terminal serine)
ANKAVNDFIL AMNYDKKKLL THQGESIENR FIKEGNQLPD EFVVIERKKR SLSTNTSDIS
VTACNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LCGDPSSGAR
VVTGKVDMVE DLIQEGSRFT ADHPGLPISY TTSFLRDNVV ATF QNSTDYV ETKVTAYRNG
DLLLDHSGEY VAQYYITWNE LSYDHQGKEV LTPKAWDRNG QDLTAHFTTS IPLKGNVRNL
SVKIREATGL AE KDQNATK W WRTVYEKTDL PLVRKRTISI WGTTEYPQVE DKVEND
SEQ ID NO. 11 - modified pneumolysin with glycosite and further
modification and His-tag (with N-terminal serine)
SANKAVNDFIL AMNYDKKKLL THQGESIENR FIKEGNQLPD EFVVIERKKR SLSTNTSDIS
VTACNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LCGDPSSGAR
VVTGKVDMVE DLIQEGSRFT ADHPGLPISY TTSFLRDNVV ATF QNSTDYV ETKVTAYRNG
DLLLDHSGEY VAQYYITWNE LSYDHQGKEV LTPKAWDRNG QDLTAHFTTS IPLKGNVRNL
SVKIREATGL AE KDQNATK W WRTVYEKTDL PLVRKRTISI WGTTEYPQVE DKVEND
HHHHHH
SEQ ID NO. 12 - modified pneumolysin with glycosite and further
modification and His-tag (without N-terminal serine)
ANKAVNDFIL AMNYDKKKLL THQGESIENR FIKEGNQLPD EFVVIERKKR SLSTNTSDIS
VTACNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LCGDPSSGAR
VVTGKVDMVE DLIQEGSRFT ADHPGLPISY TTSFLRDNVV ATF QNSTDYV ETKVTAYRNG
DLLLDHSGEY VAQYYITWNE LSYDHQGKEV LTPKAWDRNG QDLTAHFTTS IPLKGNVRNL
SVKIREATGL AE KDQNATK W WRTVYEKTDL PLVRKRTISI WGTTEYPQVE DKVEND
HHHHHH
SEQ ID NO: 13 - DsbA signal sequence
MKKIWLALAGLVLAFSASA
SEQ ID NO: 14 - OmpA signal sequence
MKKTAIAIAVALAGFATVAQA
SEQ ID NO: 15 - MalE signal sequence
MKIKTGARILALSALTTMMFSASALA
SEQ ID NO: 16 - PelB signal sequence
MKYLLPTAAAGLLLLAAQPAMA
SEQ ID NO: 17 - LTIIb signal sequence
MSFKKIIKAFVIMAALVSVQAHA
SEQ ID NO: 18 - XynA signal sequence
MFKFKKKFLVGLTAAFMSISMFSATASA
SEQ ID NO: 19 - Flgl signal sequence
MIKFLSALILLLVTTAAQA
SEQ ID NO: 20 - TolB signal sequence
MKQALRVAFGFLILWASVLHA
SEQ ID NO. 21 - modified pneumolysin with glycosite and further
modification and His-tag and signal sequence PelB
MKYLLPTAAAGLLLLAAQPAMA
ANKAVNDFIL AMNYDKKKLL THQGESIENR FIKEGNQLPD EFVVIERKKR SLSTNTSDIS
VTACNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LCGDPSSGAR
VVTGKVDMVE DLIQEGSRFT ADHPGLPISY TTSFLRDNVV ATF QNSTDYV ETKVTAYRNG
DLLLDHSGEY VAQYYITWNE LSYDHQGKEV LTPKAWDRNG QDLTAHFTTS IPLKGNVRNL
SVKIREATGL AE KDQNATK W WRTVYEKTDL PLVRKRTISI WGTTEYPQVE DKVEND
HHHHHH
SEQ ID NO. 22 - modified pneumolysin with glycosite and further
modification and His-tag and signal sequence TolB
MKQALRVAFGFLILWASVLHA
SANKAVNDFIL AMNYDKKKLL THQGESIENR FIKEGNQLPD EFVVIERKKR SLSTNTSDIS
VTACNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LCGDPSSGAR
VVTGKVDMVE DLIQEGSRFT ADHPGLPISY TTSFLRDNVV ATF QNSTDYV ETKVTAYRNG
DLLLDHSGEY VAQYYITWNE LSYDHQGKEV LTPKAWDRNG QDLTAHFTTS IPLKGNVRNL
SVKIREATGL AE KDQNATK W WRTVYEKTDL PLVRKRTISI WGTTEYPQVE DKVEND
HHHHHH
SEQ ID NO. 23 - fragment of modified pneumolysin sequence
[X3]TGL A[X2] KDQNATK
SEQ ID NO. 24 - fragment of modified pneumolysin sequence
[X3]TGL A[X2] KDQNATK W WRTVYEKTDL PLVRKRTISI WGTT[X2
SEQ ID NO. 25 - fragment of modified pneumolysin sequence
[X2]Y VAQYYITWNE LSYDHQGKEV LTPKAWDRNG QDLTAHFTTS IPLKGNVRN
SVKIRE[X3]TGL A[X2] KDQNATK W WRTVYEKTDL PLVRKRTISI WGTT[X2]
SEQ ID NO. 26 - fragment of modified pneumolysin sequence
[X1]GDPSSGARVVTGKVDMVE DLIQEGSRFT ADHPGLPISY TTSFLRDNVV ATF
QNSTDYV ETKVTAYRNGDLLLDHSG[X2]Y VAQYYITWNE LSYDHQGKEV LTPKAWDRNG
QDLTAHFTTS IPLKGNVRNLSVKIRE[X3]TGL A[X2] KDQNATK W WRTVYEKTDL
PLVRKRTISI WGTT[X2]
SEQ ID NO. 27 - fragment of modified pneumolysin sequence
[X1]NDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI
L[X1]GDPSSGARVVTGKVDMVE DLIQEGSRFT ADHPGLPISY TTSFLRDNVV ATF
QNSTDYV ETKVTAYRNGDLLLDHSG[X2]Y VAQYYITWNE LSYDHQGKEV LTPKAWDRNG
QDLTAHFTTS IPLKGNVRNLSVKIRE[X3]TGL A[X2] KDQNATK W WRTVYEKTDL
PLVRKRTISI WGTT[X2]
SEQ ID NO. 28 - consensus sequence
D/E-X-N-Z-S/T
SEQ ID NO. 29 - consensus sequence
D-Q-N-A-T
SEQ ID NO. 30 - consensus sequence
K-D/E-X-N-Z-S/T-K
SEQ ID NO. 31 - consensus sequence
K-D-Q-N-A-T-K
SEQ ID NO. 32 - _{PelB-ss}dPLY_{His6} as in pGVXN1979 (498 aa, harboring
5 detoxifying mutations G293V, A370E, C428A, W433E, and
L460E):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILVGDPSSG ARVVTGKVDM VEDLIQEGSR FTADHPGLPI SYTTSFLRDN |
| 361 | VVATFQNSTD YVETKVTAYR NGDLLLDHSG EYVAQYYITW NELSYDHQGK EVLTPKAWDR |
| 421 | NGQDLTAHFT TSIPLKGNVR NLSVKIREAT GLAEEWWRTV YEKTDLPLVR KRTISIWGTT |
| 481 | EYPQVEDKVE NDHHHHHH |

SEQ ID NO. 33 - _{PelB-ss}dPLY^{mut1}_{His6} as in pGVXN2193 (504 aa, G2931,
A370E, C428A, W433E, L460E, K4KDQNATK):

| | |
|---|---|
| 1 | MKYLLPTAAA GLLLLAAQPA MAANKDQNAT KAVNDFILAM NYDKKKLLTH QGESIENRFI |
| 61 | KEGNQLPDEF VVIERKKRSL STNTSDISVT ATNDSRLYPG ALLVVDETLL ENNPTLLAVD |
| 121 | RAPMTYSIDL PGLASSDSFL QVEDPSNSSV RGAVNDLLAK WHQDYGQVNN VPAPMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 34 - _{PelB-ss}dPLY^{mut4}_{His6} as in pGVXN2196 (504 aa, G293V,
A370E, C428A, W433E, L460E, Q24KDQNATK):

| | |
|---|---|
| 1 | MKYLLPTAAA GLLLLAAQPA MAANKAVNDF ILAMNYDKKK LLTHKDQNAT KGESIENRFI |
| 61 | KEGNQLPDEF VVIERKKRSL STNTSDISVT ATNDSRLYPG ALLVVDETLL ENNPTLLAVD |
| 121 | RAPMTYSIDL PGLASSDSFL QVEDPSNSSV RGAVNDLLAK WHQDYGQVNN VPAPMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 35 - _{PelB-ss}dPLY^{mut5}_{His6} as in pGVXN2197 (504 aa, G293V,
A370E, C428A, W433E, L460E, S27KDQNATK):

| | |
|---|---|
| 1 | MKYLLPTAAA GLLLLAAQPA MAANKAVNDF ILAMNYDKKK LLTHQGEKEQ NATKIENRFI |
| 61 | KEGNQLPDEF VVIERKKRSL STNTSDISVT ATNDSRLYPG ALLVVDETLL ENNPTLLAVD |
| 121 | RAPMTYSIDL PGLASSDSFL QVEDPSNSSV RGAVNDLLAK WHQDYGQVNN VPAPMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 36 - _{PelB-ss}dPLY^{mut6}_{His6} as in pGVXN2198 (504 aa, G293V,
A370E, C428A, W433E, L460E, E29KDQNATK):

| | |
|---|---|
| 1 | MKYLLPTAAA GLLLLAAQPA MAANKAVNDF ILAMNYDKKK LLTHQGESIN DQNATKNRFI |
| 61 | KEGNQLPDEF VVIERKKRSL STNTSDISVT ATNDSRLYPG ALLVVDETLL ENNPTLLAVD |
| 121 | RAPMTYSIDL PGLASSDSFL QVEDPSNSSV RGAVNDLLAK WHQDYGQVNN VPAPMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 37 - _{PelB-ss}dPLY^{mut10}_{His6} as in pGVXN2202 (504 aa, G293V,
A370E, C428A, W433E, L460E, S68KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDKD QNATKRLYPG ALLVVDETLL ENNPTLLAVD |
| 121 | RAPMTYSIDL PGLASSDSFL QVEDPSNSSV RGAVNDLLAK WHQDYGQVNN VPAPMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 38 - _{PelB-ss}dPLY^{mut15}_{His6} as in pGVXN2207 (504 aa, G293V,
A370E, C428A, W433E, L460E, S109KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASK DQNATKDSFL QVEDPSNSSV RGAVNDLLAK WHQDYGQVNN VPARMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 39 - _{PelB-ss}dPLY^{mut16}_{His6} as in pGVXN2208 (504 aa, G293V,
A370E, C428A, W433E, L460E, L113KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFKDQNATK QVEDPSNSSV RGAVNDLLAK WHQDYGQVNN VPARMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 40 - _{PelB-ss}dPLY^{mut19}_{His6} as in pGVXN2211 (504 aa, G293V,
A370E, C428A, W433E, L460E, Q140KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG KDQNTAKVNN VPARMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 41 - _{PelB-ss}dPLY^{mu20}_{His6} as in pGVXN2212 (504 aa, G293V,
A370E, C428A, W433E, L460E, P145KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVKDQNA TKARMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGKYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTKYPQ VEDKVENDHH HHHH |

SEQ ID NO. 42 - _{PelB-ss}dPLY^{mu25}_{His6} as in pGVXN2217 (504 aa, G293V,
A3701E, C428A, W433E, L460E, A206KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDKDQN ATKVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAK EWWRTVYEKT DLPLVRKRTI |
| 481 | SIWGTTEYPQ VEDKVENDHH HHHH |

SEQ ID NO. 43 - _{PelB-ss}dPLY^{mu31}_{His6} as in pGVXN2223 (504 aa, G293V,
A370E, C428A, W433E, L460E, K271KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKDQNATKVA |
| 301 | PQTEWKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTKYPQ VEDKVENDHH HHHH |

SEQ ID NO. 44 - _{PelB-ss}dPLY^{mu32}_{His6} as in pGVXN2224 (504 aa, G293V,
A370E, C428A, W433E, L460E, K279DQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | DQNATKQILD NTEVKAVILV GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 45 - _{PelB-ss}dPLY^{mu33}_{His6} as in pGVXN2225 (504 aa, G293V,
A370E, C428A, W4335, L460E, P296KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILVGDKDQN ATKSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 46 - _{PelB-ss}dPLY^{mu34}_{His6} as in pGVXN2226 (504 aa, G293V,
A370E, C428A, W433E, L460E, R301KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILVGDPSSG AKDQNATKVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 47 - _{PelB-ss}dPLY^{mu35}_{His6} as in pGVXN2227 (504 aa, G293V,
A370E, C428A, W433E, L460E, G305KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILVGDPSSG ARVVTKDQNA TKKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 48 - _{PelB-ss}dPLY^{mu37}_{His6} as in pGVXN2229 (504 aa, G293V,
A370E, C428A, W433E, L460E, P325KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILVGDPSSG ARVVTGKVDM VEDLIQEGSR FTADHKDQNA TKGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 49 - _{PelB-ss}dPLY^{mu47}_{His6} as in pGVXN2239 (504 aa, G293V,
A370E, C428A, W433E, L460E, L431KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILVGDPSSG ARVVTGKVDM VEDLIQEGSR FTADHPGLPI SYTTSFLRDN |
| 361 | VVATFQNSTD YVETKVTAYR NGDLLLDHSG EYVAQYYITW NELSYDHQGK EVLTPKAWDR |
| 421 | NGQDLTAHFT TSIPLKGNVR NLSVKIREAT GKDQNATKAE EWWRTVYEKT DLPLVRKRTI |
| 481 | SIWGTTEYPQ VEDKVENDHH HHHH |

SEQ ID NO. 50 - _{PelB-ss}dPLY^{mut48}_{His6} as in pGVXN2240 (504 aa, G293V,
A370E, C428A, W433E, L460E, E434KDQQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTATNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILVGDPSSG ARVVTGKVDM VEDLIQEGSR FTADHPGLPI SYTTSFLRDN |
| 361 | VVATFQNSTD YVETKVTAYR NGDLLLDHSG EYVAQYYITW NELSYDHQGK EVLTPKAWDR |
| 421 | NGQDLTAHFT TSIPLKGNVR NLSVKIREAT GLAEKDQNAT KWWRTVYEKT DLPLVRKRTI |
| 481 | SIWGTTEYPQ VEDKVENDHH HHHH |

SEQ ID NO. 51 - _{PelB-ss}dPLY_{His6} as in pGVXN2369 (498 aa, harboring
6 detoxifying mutations T65C, G293C, A370E, C428A, W433E, and
L460E):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTACNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILVGDPSSG ARVVTGKVDM VEDLIQEGSR FTADHPGLPI SYTTSFLRDN |
| 361 | VVATFQNSTD YVETKVTAYR NGDLLLDHSG EYVAQYYITW NELSYDHQGK EVLTPKAWDR |
| 421 | NGQDLTAHFT TSIPLKGNVR NLSVKIREAT GLAEEWWRTV YEKTDLPLVR KRTISIWGTT |
| 481 | EYPQVEDKVE NDHHHHHH |

SEQ ID NO. 52 - _{PelB-ss}dPLY^{mut4}_{His6} as in pGVXN2400 (504 aa, T65C,
G293C, A370E, C428A, W433E, L460E, Q24KDQNATK):

| | |
|---|---|
| 1 | MKYLLPTAAA GLLLLAAQPA MAANKAVNDF ILAMNYDKKK LLTHKDQNAT KGESIENRFI |
| 61 | KEGNQLPDEF VVIERKKRSL STNTSDISVT ACNDSRLYPG ALLVVDETLL ENNPTLLAVD |
| 121 | RAPMTYSIDL PGLASSDSFL QVEDPSNSSV RGAVNDLLAK WHQDYGQVNN VPAPMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILC GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 53 - _{PelB-ss}dPLY^{mut48}_{His6} as in pGVXN2401 (504 aa, T65C,
G293C, A370E, C428A, W433E, L460E, E434KDQNATK):

| | |
|---|---|
| 1 | MKYLLPTAAA GLLLLAAQPA MAANKAVNDF ILAMNYDKKK LLTHQGESIE NRFIKEGNQL |
| 61 | PDEFVVIERK KRSLSTNTSD ISVTACNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILCGDPSSG ARVVTGKVDM VEDLIQEGSR FTADHPGLPI SYTTSFLRDN |
| 361 | VVATFQNSTD YVETKVTAYR NGDLLLDHSG EYVAQYYITW NELSYDHQGK EVLTPKAWDR |
| 421 | NGQDLTAHFT TSIPLKGNVR NLSVKIREAT GLAEKDQNAT KWWRTVYEKT DLPLVRKRTI |
| 481 | SIWGTTEYPQ VEDKVENDHH HHHH |

SEQ ID NO. 54 - _{DsbA-ss}dPLY^{mut4}_{His6} as in pGVXN2887 (502 aa, T65C,
G293C, A370E, C428A, W433E, L460E, Q24KDQNATK):

| | |
|---|---|
| 1 | MKKIWLALAG LVLAFSASAS ANKAVNDFIL AMNYDKKKLL THKDQNATKG ESIENRFIKE |
| 61 | GNQLPDEFVV IERKKRSLST NTSDISVTAC NDSRLYPGAL LVVDETLLEN NPTLLAVDRA |
| 121 | PMTYSIDLPG LASSDSFLQV EDPSNSSVRG AVNDLLAKWH QDYGQVNNVP ARMQYEKITA |
| 181 | HSMEQLKVKF GSDFEKTGNS LDIDFNSVHS GEKQIQIVNF KQIYYTVSVD AVKNPGDVFQ |
| 241 | DTVTVEDLKQ RGISAERPLV YISSVAYGRQ VYLKLETTSK SDEVEAAFEA LIKGVKVAPQ |
| 301 | TEWKQILDNT EVKAVILCGD PSSGARVVTG KVDMVEDLIQ EGSRFTADHP GLPISYTTSF |
| 361 | LRDNVVATFQ NSTDYVETKV TAYRNGDLLL DHSGEYVAQY YITWNELSYD HQGKEVLTPK |
| 421 | AWDRNGQDLT AHFTTSIPLK GNVRNLSVKI REATGLAEEW WRTVYEKTDL PLVRKRTISI |
| 481 | WGTTFYPQVE DKVENDHHHH HH |

SEQ ID NO. 55 - _{DsbA-ss}dPLY^{mut48}_{His6} as in pGVXN2895 (502 aa, T65C,
G293C, A370E, C428A, W433E, L460E, E434KDQNATK):

| | |
|---|---|
| 1 | MKKIWLALAG LVLAFSASAS ANKAVNDFIL AMNYDKKKLL THQGESIENR FIKEGNQLPD |
| 61 | EFVVIERKKR SLSTNTSDIS VTACNDSRLY PGALLVVDET LLENNPTLLA VDRAPMTYSI |
| 121 | DLPGLASSDS FLQVEDPSNS SVRGAVNDLL AKWHQDYGQV NNVPARMQYE KITAHSMEQL |
| 181 | KVKFGSDFEK TGNSLDIDFN SVHSGEKQIQ IVNFKQIYYT VSVDAVKNPG DVFQDTVTVE |
| 241 | DLKQRGISAE RPLVYISSVA YGRQVYLKLE TTSKSDEVEA AFEALIKGVK VAPQTEWKQI |
| 301 | LDNTEVKAVI LCGDPSSGAR VVTGKVDMVE DLIQEGSRFT ADHPGLPISY TTSFLRDNVV |
| 361 | ATFQNSTDYV ETKVTAYRNG DLLLDHSGEY VAQYYITWNE LSYDHQGKEV LTPKAWDRNG |
| 421 | QDLTAHFTTS IPLKGNVRNL SVKIREATGL AEKDQNATKW WRTVYEKTDL PLVRKRTISI |
| 481 | WGTTEYPQVE DKVENDHHHH HH |

SEQ ID NO. 56 - _{Flg1-ss}dPLY^{mut4}_{His6} as in pGVXN2888 (502 aa, T65C,
G293C , A370E, C428A, W433E, L460E, Q24KDQNATK):

| | |
|---|---|
| 1 | MIKFLSALIL LLVTTAAQAS ANKAVNDFIL AMNYDKKKLL THKDQNATKG ESIENRFIKE |
| 61 | GNQLPDEFVV IERKKRSLST NTSDISVTAC NDSRLYPGAL LVVDETLLEN NPTLLAVDRA |
| 121 | PMTYSIDLPG LASSDSFLQV EDPSNSSVRG AVNDLLAKWH QDYGQVNNVP ARMQYEKITA |
| 181 | HSMEQLKVKF GSDFEKTGNS LDIDFNSVHS GEKQIQIVNF KQIYYTVSVD AVKNPGDVFQ |
| 241 | DTVTVEDLKQ RGISAERPLV YISSVAYGRQ VYLKLETTSK SDEVEAAFEA LIKGVKVAPQ |
| 301 | TEWKQILDNT EVKAVILCGD PSSGARVVTG KVDMVEDLIQ EGSRFTADHP GLPISYTTSF |
| 361 | LRDNVVATFQ NSTDYVETKV TAYRNGDLLL DHSGEYVAQY YITWNELSYD HQGKEVLTPK |
| 421 | AWDRNGQDLT AHFTTSIPLK GNVRNLSVKI REATGLAFEW WRTVYEKTDL PLVRKRTISI |
| 481 | WGTTEYPQVE DKVENDHHHH HH |

SEQ ID NO. 57 - _{Flg1-ss}dPLY^{mut48}_{His6} as in pGVXN2896 (502 aa, T65C,
G293C, A370E, C428A, W433E, L460E, E434KDQNATK):

| | |
|---|---|
| 1 | MIKFLSALIL LLVTTAAQAS ANKAVNDFIL AMNYDKKKLL THQGESIENR FIKEGNQLPD |
| 61 | EFVVIERKKR SLSTNTSDIS VTACNDSRLY PGALLVVDET LLENNPTLLA VDRAPMTYSI |
| 121 | DLPGLASSDS FLQVEDPSNS SVRGAVNDLL AKWHQDYGQV NNVPARMQYE KITAHSMEQL |
| 181 | KVKFGSDFEK TGNSLDIDFN SVHSGEKQIQ IVNFKQIYYT VSVDAVKNPG DVFQDTVTVE |
| 241 | DLKQRGISAE RPLVYISSVA YGRQVYLKLE TTSKSDEVEA AFEALIKGVK VAPQTEWKQI |
| 301 | LDNTEVKAVI LCGDPSSGAR VVTGKVDMVE DLIQEGSRFT ADHPGLPISY TTSFLRDNVV |
| 361 | ATFQNSTDYV ETKVTAYRNG DLLLDHSGEY VAQYYITWNE LSYDHQGKEV LTPKAWDRNG |
| 421 | QDLTAHFTTS IPLKGNVRNL SVKIREATGL AEKDQNATKW WRTVYEKTDL PLVRKRTISI |
| 481 | WGTTEYPQVE DKVENDHHHH HH |

SEQ ID NO. 58 - _{LTIIb-ss}dPLY^{mut4}_{His6} as in pGVXN2889 (506 aa, T65C,
G293C, A370E, C428A, W433E, L460E, Q24KDQNATK):

| | |
|---|---|
| 1 | MSFKKIIKAF VIMAALVSVQ AHASANKAVN DFILAMNYDK KKLLTHKDQN ATKGESIENR |
| 61 | FIKEGNQLPD EFVVIERKKR SLSTNTSDIS VTACNDSRLY PGALLVVDET LLENNPTLLA |
| 121 | VDRAPMTYSI DLPGLASSDS FLQVEDPSNS SVRGAVNDLL AKWHQDYGQV NNVPAPMQYE |
| 181 | KITAHSMEQL KVKFGSDFEK TGNSLDIDFN SVHSGEKQIQ IVNFKQIYYT VSVDAVKNPG |
| 241 | DVFQDTVTVE DLKQRGISAE RPLVYISSVA YGRQVYLKLE TTSKSDEVEA AFEALIKGVK |
| 301 | VAPQTEWKQI LDNTEVKAVI LCGDPSSGAR VVTGKVDMVE DLIQEGSRFT ADHPGLPISY |
| 361 | TTSFLRDNVV ATFQNSTDYV ETKVTAYRNG DLLLDHSGEY VAQYYITWNE LSYDHQGKEV |
| 421 | LTPKAWDRNG QDLTAHFTTS IPLKGNVRNL SVKIREATGL AEEWWRTVYE KTDLPLVRKR |
| 481 | TISIWGTTEY PQVEDKVEND HHHHHH |

SEQ ID NO. 59 - _{LTIIb-ss}dPLY^{mut48}_{His6} as in pGVXN2897 (506 aa, T65C,
G293C, A370E, C428A, W433E, L460E, E434KDQNATK):

| | |
|---|---|
| 1 | MSFKKIIKAF VIMAALVSVQ AHASANKAVN DFILAMNYDK KKLLTHQGES IENRFIKEGN |
| 61 | QLPDEFVVIE RKKRSLSTNT SDISVTACND SRLYPGALLV VDETLLENNP TLLAVDRAPM |
| 121 | TYSIDLPGLA SSDSFLQVED PSNSSVRGAV NDLLAKWHQD YGQVNNVPAR MQYEKITAHS |
| 181 | MEQLKVKFGS DFEKTGNSLD IDFNSVHSGE KQIQIVNFKQ IYYTVSVDAV KNPGDVFQDT |
| 241 | VTVEDLKQRG ISAERPLVYI SSVAYGRQVY LKLETTSKSD EVEAAFEALI KGVKVAPQTE |
| 301 | WKQILDNTEV KAVILCGDPS SGARVVTGKV DMVEDLIQEG SRFTADHPGL PISYTTSFLR |
| 361 | DNVVATFQNS TDYVETKVTA YRNGDLLLDH SGEYVAQYYI TWNELSYDHQ GKEVLTPKAW |
| 421 | DRNGQDLTAH FTTSIPLKGN VRNLSVKIRE ATGLAEKDQN IAKWWRTVYE KTDLPLVRKR |
| 481 | TISIWGTTEY PQVEDKVEND HHHHHH |

SEQ ID NO. 60 - _{MalE-ss}dPLY^{mut4}_{His6} as in pGVXN2890 (509 aa, T65C,
G293C, A370E, C428A, W433E, L460E, Q24KDQNATK):

| | |
|---|---|
| 1 | MKIKTGARIL ALSALTTMMF SASALASANK AVNDFILAMN YDKKKLLTHK DQNATKGESI |
| 61 | ENRFIKEGNQ LPDEFVVIER KKRSLSTNTS DISVTACNDS RLYPGALLVV DETLLENNPT |
| 121 | LLAVDRAPMT YSIDLPGLAS SDSFLQVEDP SNSSVRGAVN DLLAKWHQDY GQVNNVPARM |
| 181 | QYEKITAHSM EQLKVKFGSD FEKTGNSLDI DFNSVHSGEK QIQIVNFKQI YYTVSVDAVK |
| 241 | NPGDVFQDTV TVEDLKQRGI SAERPLVYIS SVAYGRQVYL KLETTSKSDE VEAAFEALIK |
| 301 | GVKVAPQTEW KQILDNTEVK AVILCGDPSS GARVVTGKVD MVEDLIQEGS RFTADHPGLP |
| 361 | ISYTTSFLRD NVVATFQNST DYVETKVTAY RNGDLLLDHS GEYVAQYYIT WNELSYDHQG |
| 421 | KEVLTPKAWD RNGQDLTAHF TTSIPLKGNV RNLSVKIREA TGLAEEWWRT VYEKTDLPLV |
| 481 | RKRTISIWGT TEYPQVEDKV ENDHHHHHH |

SEQ ID NO. 61 - _{MalE-ss}dPLY^{mut48}_{His6} as in pGVXN2898 (509 aa, T65C,
G293C, A370E, C428A, W433E, L460E, E434KDQNATK):

| | |
|---|---|
| 1 | MKIKTGARIL ALSALTTMMF SASALASANK AVNDFILAMN YDKKKLLTHQ GESIENRFIK |
| 61 | EGNQLPDEFV VIERKKRSLS TNTSDISVTA CNDSRLYPGA LLVVDETLLE NNPTLLAVDR |
| 121 | APMTYSIDLP GLASSDSFLQ VEDPSNSSVR GAVNDLLAKW HQDYGQVNNV PARMQYEKIT |
| 181 | AHSMEQLKVK FGSDFEKTGN SLDIDFNSVH SGEKQIQIVN FKQIYYTVSV DAVKNPGDVF |
| 241 | QDTVTVEDLK QRGISAERPL VYISSVAYGR QVYLKLETTS KSDEVEAAFE ALIKGVKVAP |
| 301 | QTEWKQILDN TEVKAVILCG DPSSGARVVT GKVDMVEDLI QEGSRFTADH PGLPISYTTS |
| 361 | FLRDNVVATF QNSTDYVETK VTAYRNGDLL LDHSGEYVAQ YYITWNELSY DHQGKEVLTP |
| 421 | KAWDRNGQDL TAHFTTSIPL KGNVRNLSVK IREATGLAEK DQNATKWWRT VYEKTDLPLV |
| 481 | RKRTISIWGT TEYPQVEDKV ENDHHHHHH |

SEQ ID NO. 62 - _{SipA-ss}dPLY^{mut4}_{His6} as in pGVXN2891 (508 aa, T65C,
G293C, A370E, C428A, W433E, L460E, Q24KDQNATK):

| | |
|---|---|
| 1 | MKMNKKVLLT STMAASLLSV ASVQASANKA VNDFILAMNY DKKKLLTHKD QNATKGESIE |
| 61 | NRFIKEGNQL PDEFVVIERK KRSLSTNTSD ISVTACNDSR LYPGALLVVD ETLLENNPTL |
| 121 | LAVDRAPMTY SIDLPGLASS DSFLQVEDPS NSSVRGAVND LLAKWHQDYG QVNNVPAPMQ |
| 181 | YEKITAHSME QLKVKFGSDF EKTGNSLDID FNSVHSGEKQ IQIVNFKQIY YTVSVDAVKN |
| 241 | PGDVFQDTVT VEDLKQRGIS AERPLVYISS VAYGRQVYLK LETTSKSDEV EAAFEALIKG |
| 301 | VKVAPQTEWK QILDNTEVKA VILCGDPSSG ARVVTGKVDM VEDLIQEGSR FTADHPGLPI |
| 361 | SYTTSFLRDN VVATFQNSTD YVETKVTAYR NGDLLLDHSG EYVAQYYITW NELSYDHQGK |
| 421 | EVLTPKAWDR NGQDLTAHFT TSIPLKGNVR NLSVKIREAT GLAFEWWRTV YEKTDLPLVR |
| 481 | KRTISIWGTT EYPQVEDKVE NDHHHHHH |

SEQ ID NO. 63 - _{SipA-ss}dPLY^{mut48}_{His6} as in pGVXN2899 (508 aa, T65C,
G293C, A370E, C428A, W433E, L460E, E434KDQNATK):

| | |
|---|---|
| 1 | MKMNKKVLLT STMAASLLSV ASVQASANKA VNDFILAMNY DKKKLLTHQG ESIENRFIKE |
| 61 | GNQLPDEFVV IERKKRSLST NTSDISVTAC NDSRLYPGAL LVVDETLLEN NPTLLAVDRA |
| 121 | PMTYSIDLPG LASSDSFLQV EDPSNSSVRG AVNDLLAKWH QDYGQVNNVP APMQYEKITA |
| 181 | HSMEQLKVKF GSDFEKTGNS LDIDFNSVHS GEKQIQIVNF KQIYYTVSVD AVKNPGDVFQ |
| 241 | DTVTVEDLKQ RGISAERPLV YISSVAYGRQ VYLKLETTSK SDEVEAAFEA LIKGVKVAPQ |
| 301 | TEWKQILDNT EVKAVILCGD PSSGARVVTG KVDMVEDLIQ EGSRFTADHP GLPISYTTSF |
| 361 | LRDNVVATFQ NSTDYVETKV TAYRNGDLLL DHSGEYVAQY YITWNELSYD HQGKEVLTPK |
| 421 | AWDRNGQDLT AHFTTSIPLK GNVRNLSVKI REATGLAEKD QNATKWWRTV YEKTDLPLVR |
| 481 | KRTISIWGTT EYPQVEDKVE NDHHHHHH |

SEQ ID NO. 64 - _{XynA-ss}dPLY^{mut4}_{His6} as in pGVXN2892 (511 aa, T65C,
G293C, A370E, C428A, W433E, L460E, Q24KDQNATK):

| | |
|---|---|
| 1 | MFKFKKKFLV GLTAAFMSIS MFSATASASA NKAVNDFILA MNYDKKKLLT HKDQNATKGE |
| 61 | SIENRFIKEG NQLPDEFVVI ERKKRSLSTN TSDISVTACN DSRLYPGALL VVDETLLENN |
| 121 | PTLLAVDRAP MTYSIDLPGL ASSDSFLQVE DPSNSSVRGA VNDLLAKWHQ DYGQVNNVPA |
| 181 | RMQYEKITAH SMEQLKVKFG SDFEKTGNSL DIDFNSVHSG EKQIQIVNFK QIYYTVSVDA |
| 241 | VKNPGDVFQD TVTVEDLKQR GISAERPLVY ISSVAYGRQV YLKLETTSKS DEVEAAFEAL |
| 301 | IKGVKVAPQT EWKQILDNTE VKAVILCGDP SSGARVVTGK VDMVEDLIQE GSRFTADHPG |
| 361 | LPISYTTSFL RDNVVATFQN STDYVETKVT AYRNGDLLLD HSGEYVAQYY ITWNELSYDH |
| 421 | QGKEVLTPKA WDRNGQDLTA HFTTSIPLKG NVRNLSVKIR EATGLAEEWW RTVYEKTDLP |
| 481 | LVRKRTISIW GTTEYPQVED KVENDHHHHHH |

SEQ ID NO. 65 - _{XynA-ss}dPLY^{mut48}_{His6} as in pGVXN2900 (511 aa, T65C,
G2930, A370E, C428A, W433E, L460E, E434KDQNATK):

| | |
|---|---|
| 1 | MFKFKKKFLV GLTAAFMSIS MFSATASASA NKAVNDFILA MNYDKKKLLT HQGESIENRF |
| 61 | IKEGNQLPDE FVVIERKKRS LSTNTSDISV TACNDSRLYP GALLVVDETL LENNPTLLAV |
| 121 | DRAPMTYSID LPGLASSDSF LQVEDPSNSS VRGAVNDLLA KWHQDYGQVN NVPARMQYEK |
| 181 | ITAHSMEQLK VKFGSDFEKT GNSLDIDFNS VHSGEKQIQI VNFKQIYYTV SVDAVKNPGD |
| 241 | VFQDTVTVED LKQRGISAER PLVYISSVAY GRQVYLKLET TSKSDEVEAA FEALIKGVKV |
| 301 | APQTEWKQIL DNTEVKAVIL CGDPSSGARV VTGKVDMVED LIQEGSRFTA DHPGLPISYT |
| 361 | TSFLRDNVVA TFQNSTDYVE TKVTAYRNGD LLLDHSGEYV AQYYITWNEL SYDHQGKEVL |
| 421 | TPKAWDRNGQ DLTAHFTTSI PLKGNVRNLS VKIREATGLA EKDQNATKWW RTVYEKTDLP |
| 481 | LVRKRTISIW GTTEYPQVED KVENDHHHHHH |

SEQ ID NO. 66 - _{TolB-ss}dPLY^{mut4}_{His6} as in pGVXN2893 (504 aa, T65C,
G293C, A370E, C428A, W433E, L460E, Q24KDQNATK):

| | |
|---|---|
| 1 | MKQALRVAFG FLILWASVLH ASANKAVNDF ILAMNYDKKK LLTHKDQNAT KGESIENRFI |
| 61 | KEGNQLPDEF VVIERKKRSL STNTSDISVT ACNDSRLYPG ALLVVDETLL ENNPTLLAVD |
| 121 | RAPMTYSIDL PGLASSDSFL QVEDPSNSSV RGAVNDLLAK WHQDYGQVNN VPAPMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILC GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 67 - _{TolB-ss}dPLY^{mut48}_{His6} as in pGVXN2901 (504 aa, T65C,
G293C, A370E, C428A, W433E, L460E, E434KDQNATK):

| | |
|---|---|
| 1 | MKQALRVAFG FLILWASVLH ASANKAVNDF ILAMNYDKKK LLTHQGESIE NRFIKEGNQL |
| 61 | PDEFVVIERK KRSLSTNTSD ISVTACNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILCGDPSSG ARVVTGKVDM VEDLIQEGSR FTADHPGLPI SYTTSFLRDN |
| 361 | VVATFQNSTD YVETKVTAYR NGDLLLDHSG EYVAQYYITW NELSYDHQGK EVLTPKAWDR |
| 421 | NGQDLTAHFT TSIPLKGNVR NLSVKIREAT GLAEKDQNAT KWWRTVYEKT DLPLVRKRTI |
| 481 | SIWGTTEYPQ VEDKVENDHH HHHH |

SEQ ID NO. 68 - _{OmpA-ss}dPLY^{mut4}_{His6} as in pGVXN2894 (504 aa, T65C,
G293C, A370E, C428A, W433E, L460E, Q24KDQNATK):

| | |
|---|---|
| 1 | MKKTAIAIAV ALAGFATVAQ ASANKAVNDF ILAMNYDKKK LLTHKDQNAT KGESIENRFI |
| 61 | KEGNQLPDEF VVIERKKRSL STNTSDISVT ACNDSRLYPG ALLVVDETLL ENNPTLLAVD |
| 121 | RAPMTYSIDL PGLASSDSFL QVEDPSNSSV RGAVNDLLAK WHQDYGQVNN VPAPMQYEKI |
| 181 | TAHSMEQLKV KFGSDFEKTG NSLDIDFNSV HSGEKQIQIV NFKQIYYTVS VDAVKNPGDV |
| 241 | FQDTVTVEDL KQRGISAERP LVYISSVAYG RQVYLKLETT SKSDEVEAAF EALIKGVKVA |
| 301 | PQTEWKQILD NTEVKAVILC GDPSSGARVV TGKVDMVEDL IQEGSRFTAD HPGLPISYTT |
| 361 | SFLRDNVVAT FQNSTDYVET KVTAYRNGDL LLDHSGEYVA QYYITWNELS YDHQGKEVLT |
| 421 | PKAWDRNGQD LTAHFTTSIP LKGNVRNLSV KIREATGLAE EWWRTVYEKT DLPLVRKRTI |
| 481 | SIWGTTEYPQ VEDKVENDHH HHHH |

SEQ ID NO. 69 - _{OmpA-ss}dPLY^{mut48}_{His6} as in pGVXN2902 (504 aa, T65C,
G293C, A370E, C428A, W433E, L460E, E434KDQNATK):

| | |
|---|---|
| 1 | MKKTAIAIAV ALAGFATVAQ ASANKAVNDF ILAMNYDKKK LLTHQGESIE NRFIKEGNQL |
| 61 | PDEFVVIERK KRSLSTNTSD ISVTACNDSR LYPGALLVVD ETLLENNPTL LAVDRAPMTY |
| 121 | SIDLPGLASS DSFLQVEDPS NSSVRGAVND LLAKWHQDYG QVNNVPARMQ YEKITAHSME |
| 181 | QLKVKFGSDF EKTGNSLDID FNSVHSGEKQ IQIVNFKQIY YTVSVDAVKN PGDVFQDTVT |
| 241 | VEDLKQRGIS AERPLVYISS VAYGRQVYLK LETTSKSDEV EAAFEALIKG VKVAPQTEWK |
| 301 | QILDNTEVKA VILCGDPSSG ARVVTGKVDM VEDLIQEGSR FTADHPGLPI SYTTSFLRDN |
| 361 | VVATFQNSTD YVETKVTAYR NGDLLLDHSG EYVAQYYITW NELSYDHQGK EVLTPKAWDR |
| 421 | NGQDLTAHFT TSIPLKGNVR NLSVKIREAT GLAEKDQNAT KWWRTVYEKT DLPLVRKRTI |
| 481 | SIWGTTEYPQ VEDKVENDHH HHHH |

SEQ ID NO: 70 - SipA signal sequence

| | |
|---|---|
| 1 | MKMNKKVLLT STMAASLLSV ASVQAS |

SEQ ID NO: 71 - pneumolysin, Serotype7F_CDC1087-00

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKIMAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFESLIKGV APQTEWKQIL DNTEVKAVIL GGDPSSGARV |
| 301 | VTGKVDMVED LIQEGSRFTA DHPGLPISYT TSFLRDNVVA TFQNSTDYVE TKVTAYRNGD |
| 361 | LLLDHSGAYV AQYYITWNEL SYDHQGKEVL TPKAWDRNGQ DLTAHFTTSI PLKGNVRNLS |
| 421 | VKIRECTGLA WEWWRTVYEK TDLPLVRKRT ISIWGTTLYP QVEDKVEND |

SEQ ID NO: 72 - pneumolysin, Serotype9_SP195

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALMKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWD ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 73 - pneumolysin, Serotype6A_CDC1873-00

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYIASV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWD ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 74 - pneumolysin, Serotype2_R6

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWD ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 75 - pneumolysin, Serotype6B_670-6B

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWD ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 76 - pneumolysin, Serotype23F_ATCC_700669

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWD ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 77 - pneumolysin, Serotype4_TIGR4

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWN ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 78 - pneumolysin, Serotype5_70585

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWN ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 79 - pneumolysin, Serotype14_JJA

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWN ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 80 - pneumolysin, serotype1_INV104

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWN ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 81 - pneumolysin, serotype11A_AP200

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWN ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 82 - pneumolysin, serotype19F_G54

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWN ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 83 - pneumolysin, Serotype3_OXC14

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWN ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 84 - pneumolysin, serotype12F_CDC0288-04

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWN ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 85 - pneumolysin, Serotype19A_CDC3059-06

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWN ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 86 - pneumolysin, Serotype18C_SP18-B574

| | |
|---|---|
| 1 | MANKAVNDFI LAMNYDKKKL LTHQGESIEN RFIKEGNQLP DEFVVIERKK RSLSTNTSDI |
| 61 | SVTATNDSRL YPGALLVVDE TLLENNPTLL AVDRAPMTYS IDLPGLASSD SFLQVEDPSN |
| 121 | SSVRGAVNDL LAKWHQDYGQ VNNVPARMQY EKITAHSMEQ LKVKFGSDFE KTGNSLDIDF |
| 181 | NSVHSGEKQI QIVNFKQIYY TVSVDAVKNP GDVFQDTVTV EDLKQRGISA ERPLVYISSV |
| 241 | AYGRQVYLKL ETTSKSDEVE AAFEALIKGV KVAPQTEWKQ ILDNTEVKAV ILGGDPSSGA |
| 301 | RVVTGKVDMV EDLIQEGSRF TADHPGLPIS YTTSFLRDNV VATFQNSTDY VETKVTAYRN |
| 361 | GDLLLDHSGA YVAQYYITWN ELSYDHQGKE VLTPKAWDRN GQDLTAHFTT SIPLKGNVRN |
| 421 | LSVKIRECTG LAWEWWRTVY EKTDLPLVRK RTISIWGTTL YPQVEDKVEN D |

SEQ ID NO: 87 - pneumolysin protein wild type sequence (with N-
terminal methionine)
MANKAVNDFIL AMNYDKKKLL THQGESIENR FIKEGNQLPD EFVVIERKKR SLSTNTSDIS
VTATNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LGGDPSSGAR
VVTGKVDMVE DLIQEGSRFT ADHPGLPISY TTSFLRDNVV ATF QNSTDYV ETKVTAYRNG
DLLLDHSGAY VAQYYITWNE LSYDHQGKEV LTPKAWDRNG QDLTAHFTTS IPLKGNVRNL
SVKIRECTGL AW E W WRTVYEKTDL PLVRKRTISI WGTTLYPQVE DKVEND
SEQ ID NO: 88 - pneumolysin sequence (without N-terminal methionine)
ANKAVNDFIL AMNYDKKKLL THQGESIENR FIKEGNQLPD EFVVIERKKR SLSTNTSDIS
VTATNDSRLY PGALLVVDET LLENNPTLLA VDRAPMTYSI DLPGLASSDS FLQVEDPSNS
SVRGAVNDLL AKWHQDY GQV NNVPARMQYE KITAHSMEQL KVKFGSDFEK TGNSLDIDFN
SVHSGEKQIQ IVNFKQIYYT VSVDA VKNPG DVFQDTVTVE DLKQRGISAE RPLVYISSVA
YGRQVYLKLE TTSKSDEVEA AFEALIKGVK VAPQT EWKQI LDNTEVKAVI LGGDPSSGAR
VVTGKVDMVE DLIQEGSRFT ADHPGLPISY TTSFLRDNVV ATF QNSTDYV ETKVTAYRNG
DLLLDHSGAY VAQYYITWNE LSYDHQGKEV LTPKAWDRNG QDLTAHFTTS IPLKGNVRNL
SVKIRECTGL AW E W WRTVYEKTDL PLVRKRTISI WGTTLYPQVE DKVEND
SEQ ID NO. 89 wfdK E. coli O167
MTVIAIVVTFNRCALLKKVLHSLLSQSIALNKIIIIDNDSNDDT
AKVVHDFSEVDDIFYYYNTGDNLGGAGGFYQGFKIAEQLYYDYLWLMDDDLLPEPDCL
EKLIQDRPEGIVQPVRYDLDGACAEISPVEYNLQKIFCRNPKTKTVKEVISTVISDNC
REIDIAGVPFEGPLISKSVVNKVGYPNPDFFIFNDDLDYSLRTRSKGFSIKCIVDARA
TRLLKNNQKNDLKSWKGYFMLRNHYYILRNYGENKLVKNRVYLIMFYYFLKSVFSFDY
KFAKVVIFSFKDSFSLKNSKRFRP
SEQ ID NO. 90 wbeY from E. coli O28
MSITNKTIALVIVTYNRCNLLMEMLSSIENMSVKPDIVYVIDNN
SSDNTSSVVTECDSRKNINIKYHNTGYNAGGAGGFYIGSKMAYEDGWDRIWLADDDIV
LDKECLSNAMEYDDGRTILQPMRYNMDGSCAEISAIQYDLSNPFYLRPKRKTVQNIFN
KNILSYDIQSIPFEGPIIPREVFNVIGFPDERFFIFNDDLDFAIRAQRAGFSIKCITN
AKIVRKIPFVQSVALKTWKGYFMFRNYFRVQKVYGLSPLIYLRILLVFCLALGHSLVR
MDINSIKMLCGALKDGLSQEFKLTEKYKP
SEQ ID NO. 91 wzg from S. pneumoniae CP4
MSRRFKKSRSQKVKRSVNIVLLTIYLLLVCFLLFLIFKYNILAF
RYLNLVVTALVLLVALVGLLLIIYKKAEKFTIFLLVFSILVSSVSLFAVQQFVGLTNR
LNATSNYSEYSISVAVLADSEIENVTQLTSVTAPTGTNNENIQKLLADIKSSQNTDLT
VNQSSSYLAAYKSLIAGETKAIVLNSVFENIIESEYPDYASKIKKIYTKGFTKKVEAP
KTSKSQSFNIYVSGIDTYGPISSVSRSDVNILMTVNRDTKKILLTTTPRDAYVPIADG
GNNQKDKLTHAGIYGVDSSIHTLENLYGVDINYYVRLNFTSFLKLIDLLGGIDVYNDQ
EFTAHTNGKYYPAGNVHLDSEQALGFVRERYSLADGDRDRGRHQQKVIVAILQKLTST
EVLKNYSTIINSLQDSIQTNMPLETMINLVNAQLESGGNYKVNSQDLKGTGRMDLPSY
AMPDSNLYVMEIDDSSLAVVKAAIQDVMEGR
SEQ ID NO. 92 wzh from S. pneumoniae CP4
MIDIHSHIVFDVDDGPKSREESKALLAESYRQGVRTIVSTSHRR
KGMFETPEEKIAENFLQVREIAKEVASDLVIAYGAEIYYTPDVLDKLEKKRIPTLNDS
RYALIEFSMNTPYRDIHSALSKILMLGITPVIAHIERYDALENNEKRVRELIDMGCYT
QVNSSHVLKPKLFGERYKFMKKRAQYFLEQDLVHVIASDMHNLDGRPPHMAEAYDLVT
QKYGEAKAQELFIDNPRKIVMDQLI
SEQ ID NO. 93 wzd from S. pneumoniae CP4
MMKEQNTIEIDVFQLVKSLWKRKLMILIVALVTGAGAFAYSTFI
VKPEYTSTTRIYVVNRNQGDKPGLTNQDLQAGTYLVKDYREIILSQDVLEEVVSDLKL
DLTPKGLANKIKVTVPVDTRIVSISVNDRVPEEASRIANSLREVAAQKIISITRVSDV
TTLEEARPAISPSSPNIKRNTLIGFLAGVIGTSVIVLHLELLDTRVKRPEDIENTLQM
TLLGVVPNLGKLK
SEQ ID NO. 94 wze from S. pneumoniae CP4
MPTLEIAQKKLEFIKKAEEYYNALCTNIQLSGDKLKVISVTSVN
PGEGKTTTSINIAWSFARAGYKTLLIDGDTRNSVMLGVFKSREKITGLTEFLSGTADL
SHGLCDTNIENLFVVQSGSVSPNPTALLQSKNFNDMIETLRKYFDYIIIDTPPIGIVI
DAAIITQKCDASILVTATGEANKRDIQKAKQQLKQTGKLFLGVVLNKLDISVNKYGVY
GSYGNYGKK
SEQ ID NO. 95 wciI from S. pneumoniae CP4
MKNGNRIYSWRLFMYGIIKRLGDILLSLIGIIILCPVFMIIAIA
IKLDSEGPVIFKQKRFGIHKEYFYILKFRSMKIDAPKNVAPRNLYNPEQWITKVGAFL
RKTSLDELPQLFNILVGNMSIVGPRPAGINELDLIAERDKYGANDILPGLTGWAQING
RDTLSVEMKTELDGYYVKHLSLIMDIRCIVKTIPYVLKRKGIVEGSGKKES
SEQ ID NO. 96 wciJ from S. pneumoniae CP4
MKILFVCQHYKPEPFRLSDICEDLVRKGHEVSVLAGIPNYPEGK
IYADYRHNKKRREIIEGVTIYRSYTIPRKKSVVFRLLNYFSFAISSTLGVLLGRYKTK
DGSNFDCVFVNQLSPVMMAWAGMAYKKKYKKPMFLYCMDVWPDSLTVGGVKQDGLIFK
LFKFISKKVYRASDYIFVTSPSFKNYFVKQFDISEQKITYLPQYAEDLFIPDESIVNK
ESVDLTFAGNIGKAQNLETILKAASLIEKNTNLPKKIHFHFVGDGTELLSMKALAHEL
ELKNISFYGRRSLEEMPSFYKKSDAMLVSLIGDSIVSRTIPGKVQSYMAAGKPIIGAI
SGDAKIIVEEANCGYVSPERDVKQLAKNICKFSMLSIKRQRELGKKARCYYENHFSKE
QFMLELETCLERESKKE
SEQ ID NO. 97 wciK from S. pneumoniae CP4
MRVLFILSDNIYLTPYFNFYKELLKKLSISYDVIYWDKNINEII
TKQNYYRISFSGKGKLSKILGYVKFRKEIKKKLKENDYDMILPLHSIVSFILVDFLLF
SFKNRYIYDIRDYSYEKFLVYRLVQKQLVKNSLMNIVSSDGYKFFLPMGEYFTTHNLP
NMIELNEVKQLKNNSTFPIQLSYIGLIRFQEQNKKIIDFFANDSRFQLNFIGTNAGEL
REFCQEKNISNVNLVDTFQPKDTMSFYKNTDAVLNLYGNHTPLLDYALSNKLYFAALL
YKPILVCEDTYMEKVSIENGFGFVLPMKDESEKDCLALYIQNLDRKQLIKNCDNFMDR
ISLEKQKTEIELEKRILSLRKKND
SEQ ID NO. 98 wciL from S. pneumoniae CP4
MIKVLHLFTTLDSGGVESFLFNYYSHIDRKKIQFDFIVPGKEQG
FLEDKMKELGAKVYHVPLLRKKPLHQFLSLARIIKKGDYDIVHCHGYKSAIGLILSKI
IGCKIRIIHSHMAYVTENSFQKVLRKLVTILVKILATHWFACGEDSAKWLYGEKAYKD
GKIEIIFNAIDLKKYQFLSDVREKCRRELDVSNKFVLGNIARLSDQKNQSYLFNVLKE
LILIKPNVILLLVGNGEDEQKLKQKALELNLTPYVLFLGRRTDISDLLSAMDVFLLPS
KYEGLPVSLVEAQASGLQILSSDTVTQEVDVTKNISYLPINEESVLLWKDKVLSLTSE
ECNRFEINNSMTDGLYDICYQASKLLNRYQEMCVIKEI
SEQ ID NO. 99 wzy from S. pneumoniae CP4
MQTKYICRVTLVTLSFIFAFCYLFWTLDNWNNGFLISNYVPSIF
IWVCFLIIFQITGFILQKVSIYDFSVWYLILSYFFMFGLIFNEYMGFQTTLLWSPSNF
YNNEELFHSYIFIIWILFCYSVGYLFFYSDGKVHYHSEVQNYQENEEKILYNAGRILT
GVGFISRVITDSKTVLAVRAANSYSAYSEAASSGIIDDLGVLMLPGVFSLFYSDKLSR
VIKRTIFWVMLFYLILIMILTGSRKIQVFSILALVLVYTQSLGITFSKKRVLVFLIVT
VFLLNVLVVIRGHRFDLNTIGIYLFDSFSSLDFVKNILGEVFSESGLTSLTVASAVTV
VPSSIPYEYGMTFLRTILSIFPIGWLVGDFFDKASATVVINKFLGLPVGSSFVEELFW
NFGYYGGVFWSFVLGIFSGWRLNFRAFQTSKISKVIYFSVISQLLLLVRSSSIDVYRP
IMYSLIMIFIFRRLKK
SEQ ID NO. 100 wciM from S. pneumoniae CP4
MVKKIMLHGATDYGSSNYGDYLYGEIVYDLLESKGYEVSFYNPS
DFFQMYLKEYRQKQSFTKKQADAILYIPGGYFGEGHNARFRDNLIQFKRFLPLGIWAS
YFKKPIGVLGIGAGPNNDSLMNYGIKRIINHAQFITVRDRESFDSLKHLSPSAPVHET
FDLIISSKLREEKTEQLCQLKREAKDKKIILVHYNHSKKALEKFAESISLFLENNPNY
YVVVTSDSILPYEDAYYQEFRKLVRTEDCFQFKYHSPAEMTSLLKMVDVVLTCKLHVG
VVATCFNKSVIAIACHPEKTARYYGAIGELQRCESLFDSSVNSIVKKLETFHLKPITI
PSELVLKARSSLDYLDLFLEGLVRES
SEQ ID NO. 101 wcx from S. pneumoniae CP4
MKVDRISFIKNTSSLYILNIVKLLFPLLTLPYLTRVLSLDAYGM
VIYVKALIAYVQLVIDFGFMISATKNIVNACTTPSKIGRIVGDTLVEKIFLSIISILI
YTILMWQIPIMRENILFSVFYLLATVTNIFIFDFLFRGIEKMHAVAIPYIISKTIITI
LTFIVVKDDSSILWIPILEGIGNLVAAVVSYRFLHYYGIKLSFSYLSVWVKDLKESSI
YFLSNFATTIFGVFTTVISGFYLQSQEIAFWGIAMQLLSAAKSLYNPIANSLYPHMIR
TKDIQSVKSINRIMFIPIIFGVLIVLFFSNQILSIIGGEKYTVSADFLKYLLPAFVAS
FYSMIYGWPVLGAIDKVKETTMTTILASIVQTLGLGIFILSDNFSLVTLAICSSMSEV
VLWISRYLIYFKNRSLFVRSK
SEQ ID NO. 102 mnaA from S. pneumoniae CP4
MKKVVVVFGTRPEAIKMCPLVKELRTRKNIETLVCVTGQHRQML
DQVLDTFGIIPDFDLSIMKDKQTLFDVTIGILEGMKAILESEKPDLVLVHGDTSTTFA
SSLAAFYLQIPIGHVEAGLRTYDIYSPYPEEFNRQAVGVLAQYHFTPTQLSKDNLLRE
GKTPESIFVTGNTAIDALQTTIQEDYTHPELEWIGESRFILITAHRRENLGEPMRHMF
RAIRRIIEEYSDVKAIYPIHMNPRVRQIAEEELSGCERIKMIEPLEVLDFHNFLSRSY
LILTDSGGIQEEAPSLGKPVLVMRDTTERPEGIEAGTLKLVGADENNIYRHFKELLEN
DSVYQAMSQASNPYGDGTACKKIADILEGEV
SEQ ID NO. 103 fnlA from S. pneumoniae CP4
MSQFTGKTLLITGGTGSFGNAVLKRFLETDVSEIRIFSRDEKKQ
DDMRHEFQVKVPEVAGKIRFYLGDVRDLASVKNAMHGVDYVFHAAALKQVPSCEFFPV
EAVKTNILGTENVLTAAIEAGVKQVICLSTDKAAYPVNAMGTSKAMMEKIAVAKSRTV
NPEHTKICVTRYGNVLCSRGSVVPLWIEQIKQGNALTITEPSMTRFVMTLEEAVDLVL
FAFEEGKSGDILVQKAPACTIEVLAKAVSEIFASEQDIKIIGIRHGEKRYETLLTNEE
CANAIDLGDFYRVPSDNRNLNYDKYFKDGSTNRNLLTEFNSNNTDLMDVEQVKRKLLE
LDEIQTAIRDMVADEEM
SEQ ID NO. 104 fnlB from S. pneumoniae CP4
MIKNILITGAKGFVGKNLICTLEALKDGRDRTRPNLEIGEIFQY
DRDTDPILLDEYCKKADFVFHLAGVNRPQNPDEFMEGNYGFSSRLLEILEKYENTCPV
LLSSSTQASLEGRFSNSIYGQSKLAGEELFFEYGKKTGAPVLVYRFPNLYGKWCRPNY
NSAVATFCYNLAHDLPIQVNDPSVELELLYIDDLIQECLTALEGNPHRCNLDGLQILP
SPSGNYCYVPTTHRATLGEIVSLLETFKKQPDSLVMPEIPQGSFKKKLYSTYLSYLPV
DKFKFPLKMNIDERGSFTELLKTENTGQFSVNISKPGITKGQHWHHSKWEFFMVVSGR
ALIQERRIGLDENGQEYPILNFEVSGDKIEAIHMIPGYAHNIINLSDTENLITVMWAN
ESFDPRHPDTFFEQVEK
SEQ ID NO. 105 fnlC from S. pneumoniae CP4
MKIKTDYSDIHFKDNGKLKLLIIVGTRPEIIRLSSVITKCRKYF
DVILAHTGQNYDYNLNGIFFDNLGLDTPDVYMDAVGDDLGATVGNIINTSYKLMNQIK
PDALLILGDTNSCLSAIAAKRLHIPIFHMEAGNRCKDECLPEETNRRIVDVISDVNLA
YSEHARKYLHECGLPKERTYVTGSPMAEVLHKNLSAIESSDIHERLGLKKGGYILLSA
HREENIDTDKNFISLFTAINQLAEKYNMPILYSCHPRSKKRLQESGFKLDKRVIQHEP
LGFHDYNCLQMNAFVVVSDSGTLPEESSFFTSQGYPFPAVCIRTSTERPESLDKAGFI
LAGIDENSLLQAVETAVSLAQDEDFGLPVPDYVEENVSTKVVKIIQSYTGIVDKIVWR
KS
SEQ ID NO. 106 wzg from S. pneumoniae CP12F
MLIMSRRFKKSRSQKVKRSVNIVLLTIYLLLVCFLLFLIFKYNI
LAFRYLNLVVTALVLLVALVGLLLIIYKKAEKFTIFLLVFSILVSSVSLFAVQQFVGL
TNRLNATSNYSEYSISVAVLADSDIENVTQLTSVTAPTGTDNENIQKLLADIKSSQNT
DLTVDQSSSYLAAYKSLIAGETKAIVLNSVFENIIESEYPDYASKIKKIYTKGFTKKV
EAPKTSKNQSFNIYVSGIDTYGPISSVSRSDVNILMTVNRDTKKILLTTTPRDAYVPI
ADGGNNQKDKLTHAGIYGVDSSIHTLENLYGVDINYYVRLNFTSFLKMIDLLGGVDVH
NDQEFSALHGKFHFPVGNVHLDSEQALGFVRERYSLADGDRDRGRNQQKVIVAILQKL
TSTEALKNYSTIINSLQDSIQTNMPLETMINLVNAQLESGGNYKVNSQDLKGTGRTDL
PSYAMPDSNLYVLEIDDSSLAVVKAAIQDVMEGR
SEQ ID NO. 107 wzh from S. pneumoniae CP12F
MIDIHSHIVFDVDDGPKSREESKALLAESYRQGVRIIVSTSHRR
KGMFETPEEKIAENFLQVREIAKEVASDLVIAYGAEIYYTPDVLDKLEKKRIPTLNDS
RYALIEFSMNTPYRDIHSALSKILMLGITPVIAHIERYDALENNEKRVRELIDMGCYT
QVNSSHVLKSKLFGERYKFMKKRAQYFLEQDLVHVIASDMHNLDGRPPHMAEAYDLVT
QKYGEAKAQELFIDNPRKIVMDQLI
SEQ ID NO. 108 wzd from S. pneumoniae CP12F
MMKEQNTIEIDVFQLFKTLWKRKLMILLVALVTGAGAFAYSAFI
VKPEYTSTTRIYVVNRDQGDKSGLTNQDLQAGSYLVKDYREIILSQNVLEKVATNLKL
DIPAKTLARKVQVTVPVDTRIVSISVKDKQPEEASRIANSLREVAAEKIIAVTRVSDV
TTLEEARPATTPSSPNVGRNSLFGFFGGAVVTVIAVLLIELFDIRVKRPEDVEDVLQI
PLLGVVPDLDKMK
SEQ ID NO. 109 wze from S. pneumoniae CP12F
MPTLEISQAKLDFVKKAEENYNALCTNLQLSGDDLKVFSITSVK
QGEGKSTTSTNIAWAFARAGYKTLLIDGDIRNSVMLGVFKARDKITGLTEFLSGTTDL
SQGLCDTNIENLFVIQAGSVSPNPTALLQSKNFSTMLETLRKYFDYIIVDTAPVGVVI
DAAIITQKCDASILVTKAGEINRRDIQKAKEQLEHTGKPFLGVVLNKFDTSVDKYGSY
GNYGKK
SEQ ID NO. 110 wciC from S. pneumoniae CP33F
MKVTIIGQIKNKRTGLGKAINDFRDYCCNRATRVTEIDITNNFN
FLSSLFQILISDTDVYYFTPAGSVAGNIRDSLFLFFMIMKRKKIVTHFHNSAFGNVMR
QHPTLMIINRILYSKVDLIILLGEKSKIMFQQLRILDEKFKIIRNGVDGYLFIEKNEL
NKKMSDLPINIIFFSNMIREKGYEILLEVAKKMVGDEKYHFYFSGKFQDNNLKTRFIN
EIYSMNNVTYLDGVYGSDKKKLLQKMHYFVLPSYYKDETLPISMLEAMANGLYIIVSD
VGVVSEVINKETASLIEMINEETADSIIEIINQTSNKLNELDFNVSKYKQELLNENIQ
ASIYQQLERIAN
SEQ ID NO. 111 wciD from S. pneumoniae CP33F
MTKKKNTGKILTVVVPSYNAENYLQETMPTILSAKNIERVELLI
VNDGSTDRTEEIARQFEREYEGIVRVISKENCGHGSAVNAGIENAVGNYFKVVDADDW
VNTNNLEDLIVFLSEVDVDQVLSPYDKIFVNYRGDIEREEECNEFSQVENEVIYSAEE
FYTRIKQTVGMHSITVKTSLLQENNIRLSEKMFYVDMEYIVYMLPYVKKVVLFDKSIY
RYRLGTETQSISMASYIKNRDMHKQVIYHLVDFYNQMRSSAVLRRITWKLILNLIRQQ
WIIYFNLSKKEGKNSECFEFDNWLIKEGRIKKIPLYFFKAVKYIRFKVKYFLGIRK
SEQ ID NO. 112 wciE from S. pneumoniae CP33F
MRKIGKVINEYFVLRKSFTPAIARNKLFEKFWGRIGNYKIFNNI
ASNFYQYKHETIINFLEKDFSQFLKSYNFKEVSHKEIEQRKIFSMWIQGYESAPKLVQ
KTIDSQRKYAEKYGYKFVFLDENNIREYVTLPSEIVEKYENGTIDFIKYSDVVRGTLL
SKYGGVWLDSTIYVDSSRELNYLKKDFYTIRAKTHERVPKYIANGRWSAFCLSGEKQN
IVFDFLEKFHVAYFMKYDIVLDYFLIDYIIELGYRTNDLIRNYIDKVEENNQELFFLA
DNFSNQYDEKEWAGVLSTTALFKCSYKCPINEATGTYFDRLMKGEL
SEQ ID NO. 113 wciF from S. pneumoniae CP33F
MISVIVPVYNVADYLRFALDSLLEQTYKDFEVILVNDGSTDNSG
EICDEYGKLYDNIHVFHKKNGGLSDARNFGLEKSRGEFITFLDSDDYFEPYALELLIT
IQKKYDVDIVSTKGGITYSHDIYSKKLMAEDYLTVKILTNKEFLAAVYYNDEMTVSAW
GKLYKRDLFKTIFPKGKIYEDLYVVAERLLNIKTVAHTDLPIYHYYQRQGSIVNSTFS
DRQYDFFDAIDHNEAIIKKFYCGDKELLAALNAKRVIGSFILSNSAFYNSKNDITKII
RIIKPYYWEVIKNKKIPMKRKVQCVLFLLSPNYYYKIKDKMLQRGRI
SEQ ID NO. 114 wchA from S. pneumoniae CP33F
MNGKIVKPSLAIIQSFLVILLTYLLSAVREAEIVSTTAIALYIL
HYFVFYISVYGQDFFKRGYLIELVQTLKYILFFALAISISNFFLEDRFSISRRGMIYF
LTLHALLVYVLNLFIKWYWKRTYPNFKGSKKILLLTATFRVEKVLDRLIESNEVVGEL
VAVSVLDKPDFQHDCLKVVAEGEIVNFATHEVVDEVFINLPSEKYNIGELVSQFETMG
IDVTVNLNAFDRSLARNKQIREMAGLNVVTFSTTFYKTSHVIAKRIIDIMGALVGLIL
CGLVSIVLVPLIRKDGGSAIFAQTRIGKNGRQFTFYKFRSMCVDAEAKKRELMEQNTM
QGGMFKVDDDPRITKIGRFIRKTSLDELPQFYNVLKGDMSLVGTRPPTVDEYEHYTPE
QKRRLSFKPGVTGLWQVSGRSEIKNFDEVVKLDVAYIDDWTIWKDIEILLKTVKVVLM
KDGAK
SEQ ID NO. 115 wciB from S. pneumoniae CP33F
MERSRLIDVKIIVATHKEVKMPQDNSLYLPIHVGRDGKSDIGFI
GDNTGDNISSLNPYYCELTGLYWAWKNLDYNYLGLVHYRRYFTNKSQGYNENVNMDDL
ILSRANVEILLEKSDIIVPKKRKYYIETLYSHYAHTLNGEHLDLARKIIEQNSSEYLS
SFDKVMKQRSGYMFNMFIMKKELLDDYLPWLFSILDTMYEQMDLTDYTLFESRLFGRV
SELLFNVWLCQKGITPKEVPFMYMERVDLFEKGKSFLMAKFFGKKYGQSF
SEQ ID NO. 116 wclP from E. coli O21
MKRKLVDFCIISLPQHNERRDKLKNEMAKYDIECRVSHAIDGRK
LLAEKYFSLFKIRSSKMFGRGFLTPSELGCFLSHKKALTEFLASGRKWLVVLEDDVLP
KENVKYLDEMINSFCSSSVYILGGQDGLKSFSRVIMGRKSICGVRKVILGTHRWLYRT
CCYCVDIKGAERILRLMEENSFFCDDWSYIVRNAKLDNVFYGQYFSHPVNLNSSSIEA
ERLFIAEK
SEQ ID NO. 117 wclQ from E. coli O21
MMGLFMGNETVSIIMPAYNAEETIKDSILSILKQTYEDFKLYII
NDNSSDSTEHIIKSIIDERIVYLLNRNGKGVSSARNVGIAACNGRYIAFCDSDDVWFE
TKLEEQLKILSAGNYKVVCSNYEVFYADTNVIKERRFKEVITYNNMLQSNHIGNLTGI
YDSTQIGKVYQQEIGHEDYLMWLTIVKKAKLVYCIQKNLARYYIHNTGLSSNKFTAAM
WQWNIYRRVLSFSLFKSLVLFFIYSVRALAKRL
SEQ ID NO. 118 Z3206 from E. coli O157
MNDNVLLIGA SGFVGTRLLE TAIADFNIKN LDKQQSHFYP EITQIGDVRD QQALDQALAG
FDTVVLLAAE HRDDVSPTSL YYDVNVQGTR NVLAAMEKNG VKNIIFTSSV AVYGLNKHNP
DENHPHDPFN HYGKSKWQAE EVLREWYNKA PTERSLTIIR PTVIFGERNR GNVYNLLKQI
AGGKFMMVGA GTNYKSMAYV GNIVEFIKYK LKNVAAGYEV YNYVDKPDLN MNQLVAEVEQ
SLNKKIPSMH LPYPLGMLGG YCFDILSKIT GKKYAVSSVR VKKFCATTQF DATKVHSSGF
VAPYTLSQGL DRTLQYEFVH AKKDDITFVS E
SEQ ID NO. 119 galE from E. coli O157
MRVLVTGGSG YIGSHTCVQL LQNGHDVIIL DNLCNSKRSV LPVIERLGGK
HPTFVEGDIR NEALMTEILH DHAIDTVIHF AGLKAVGESV QKPLEYYDNN
VNGTLRLISA MRAANVKNFI FSSSATVYGD QPKIPYVESF PTGTPQSPYG
KSKLMVEQIL TDLQKAQPDW SIALLRYFNP VGAHPSGDMG EDPQGIPNNL
MPYIAQVAVG RRDSLAIFGN DYPTEDGTGV RDYIHVMDLA DGHVVAMEKL
ANKPGVHIYN LGAGVGNSVL DVVNAFSKAC GKPVNYHFAP RREGDLPAYW
ADASKADREL NWRVTRTLDE MAQDTWHWQS RHPQGYPD
SEQ ID NO. 120 pg1B from Campylobacter jejuni
IISNDGYAFAEGARDMIAGFHQPNDLSYYGSSLSTLTYWLYKIT
PFSFESIILYMSTFLSSLVVIPIILLANEYKRPLMGFVAALLASIANSYYNRTMSGYY
DTDMLVIVLPMFILFFMVRMILKKDFFSLIALPLFIGIYLWWYPSSYTLNVALIGLFL
IYTLIFHRKEKIFYIAVILSSLTLSNIAWFYQSTIIVILFALFALEQKRLNFVIIGIL
ASVTLIFLILSGGVDPILYQLKFYIFRSDESANLTQGFMYFNVNQTIQEVENVDLSEF
MRRISGSEIVFLFSLFGFVWLLRKHKSMIMALPILVLGFLALKGGLRFTIYSVPVMAL
GFGFLLSEFKAILVKKYSQLTSNVCIVFATILTLAPVFIHIYNYKAPTVFSQNEASLL
NQLKNIANREDYVVTWWDYGYPVRYYSDVKTLVDGGKHLGKDNFFPSFALSKDEQAAA
NMARLSVEYTEKSFYAPQNDILKTDILQAMMKDYNQSNVDLFLASLSKPDFKIDTPKT
RDIYLYMPARMSLIFSTVASFSFINLDTGVLDKPFTFSTAYPLDVKNGEIYLSNGVVL
SDDFRSFKIGDNVVSVNSIVEINSIKQGEYKITPIDDKAQFYIFYLKDSAIPYAQFIL
MDKTMFNSAYVQMFFLGNYDKNLFDLVINSRDAKVFKLKI

### EXAMPLES

### Example 1

To study the insertion of glycosylation sequons into the Pneumolysin (PLY) protein from S. pneumoniae we aimed for a structure guided approach. We identified 52 target positions in the PLY protein, and we subsequently replaced the corresponding residues with the glycosylation sequon KDQNATK (SEQ ID NO. 31) by gene synthesis. Out of these, 34 mutants did not show any expression in E. coli (data not shown). The remaining 18 PLY mutants (mutants 1, 4, 5, 6, 10, 15, 16, 19, 20, 25, 31, 32, 33, 34, 35, 37, 47, and 48 (Table 1)) did show expression in E. coli (data not shown), and were subjected to an in vivo glycosylation screening to see whether these mutants can be conjugated with the S. pneumoniae capsular polysaccharide 4 (CP4) in E. coli (see below).

In addition to the glycoengineering, we detoxified our PLY carrier protein to eliminate the lytic activity due to the cholesterol dependent pore forming properties of PLY oligomers (Tilley S. J., et al, Cell 2005 Apr. 22; 121(2):247-56). Several strategies for the detoxification of PLY have been described in the literature, and we introduced a series of point mutations by gene synthesis based on a study reported by Oloo and co-workers (Oloo E. O., et al, J Biol Chem. 2011 Apr. 8; 286(14):12133). Our final detoxified PLY (dPLY) version contained the disulfide-forming mutations T65C and G293C combined with mutations A370E, W433E, C428A, and L460E.

To reconstitute the S. pneumoniae CP4 biosynthesis pathway in E. coli, we used plasmid p803 (also referred to as pGVXN803) containing the CP4 genes between aliA and dexB. As expression host, we used E. coli strain st8011, where the waaL gene is replaced with an IPTG-inducible, codon usage optimized version of pglB from Campylobacter jejuni (W3110 waaL::pglB_{cuo}). We transformed st8011 with plasmid p803, and with plasmid p207 (also referred to as pGVXN207) expressing the GalE epimerase. To analyze the glycosylation occupancy of our 18 engineered dPLY mutants, we additionally transformed pEC415 plasmids to encode these dPLY mutants. Transformed cells were inoculated into a 5 ml TB preculture supplemented with 10 mM MgCl₂, 50 μg/mL kanamycin, 100 μg/mL trimethoprim, and 20 μg/mL tetracycline, and the cultures were shaken overnight at 37° C. The main cultures were inoculated to an OD₆₀₀ of 0.1, grown at 37° C. to an OD₆₀₀ of 0.8-0.95, before the cultures were induced with 0.1% arabinose (w/v) and 1 mM IPTG. 15 h after induction, 150 OD₆₀₀ of each culture were harvested and washed with 5 mL of 0.9% NaCl. Periplasmic extracts were prepared by incubation of resuspended cells in lysis buffer (30 mM Tris-HCl, pH 8.5; 250 mM NaCl; 1 mM EDTA; 20 mg/mL lysozyme) for 30 min at 4° C. Glycosylation of dPLY mutants in the prepared extracts was analyzed by SDS-PAGE and immunoblot analysis using and anti-His primary antibody and an HRP-conjugated secondary antibody.

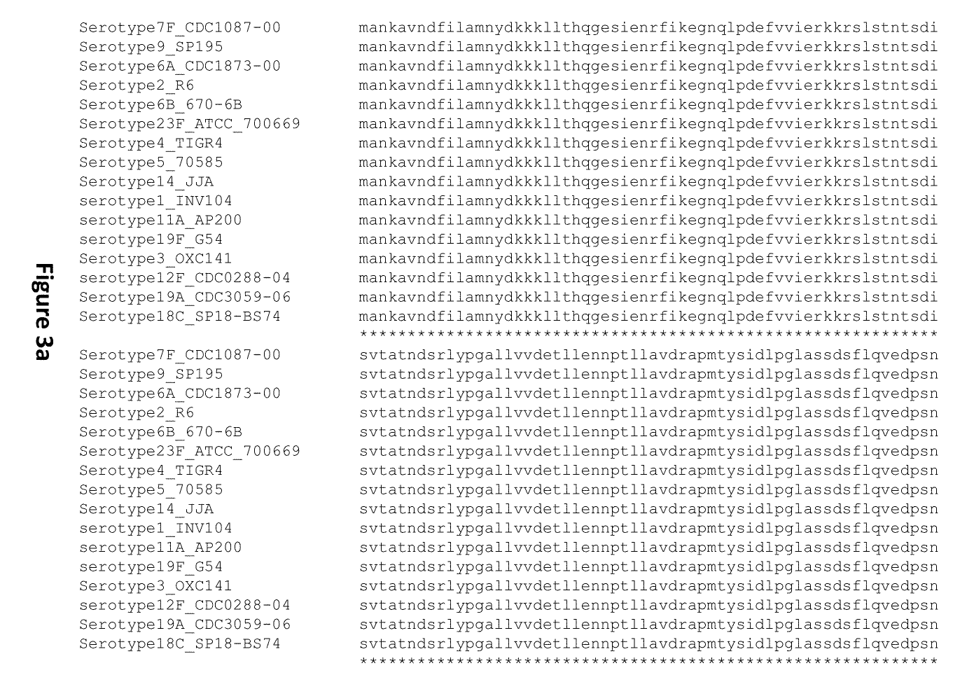

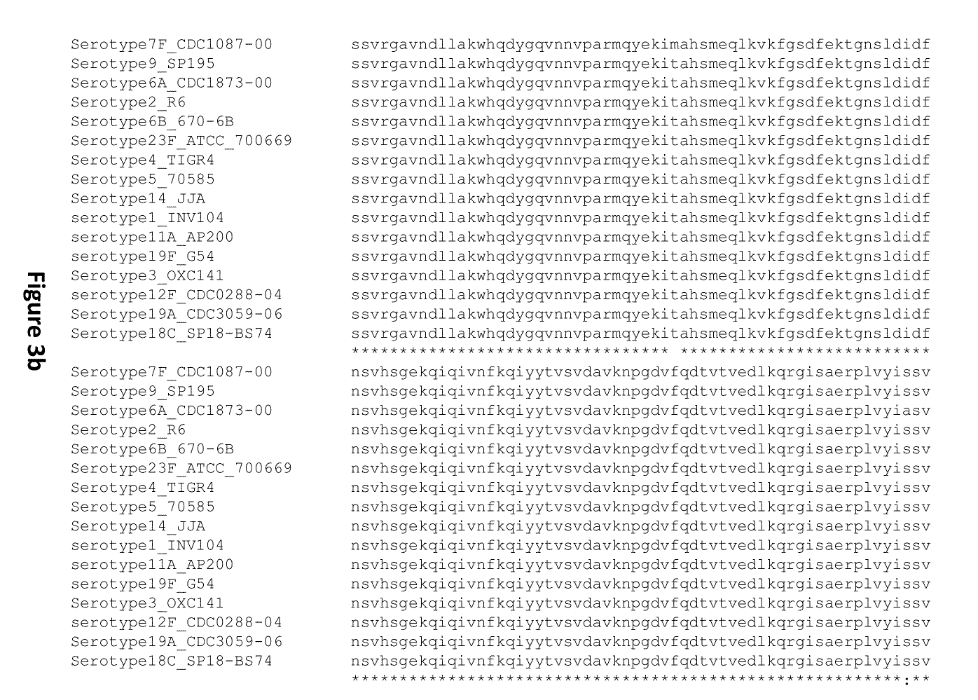

FIG. 2 shows the expression and glycosylation efficiency of dPLY mutants by PglB with the CP4 oligosaccharide. The mutants tested here encode for dPLY mutants 1, 4, 5, 6, 10, 15, 16, 19, 20, 25, 31, 32, 33, 34, 35, 37, 47, and 48. The amino acid of PLY that was replaced with the glycosylation sequon -KDQNATK- (SEQ ID NO. 31) is indicated above each lane. Glycosylation results in mobility shift to higher molecular weight and can be observed as a ladder-like pattern. From FIG. 2 it is evident that dPLY mutants 4 (replacement of Q24), 5 (replacement of S27), 6 (replacement of E29), 47 (replacement of L431), and 48 (replacement of E434) are the most efficient substrates for glycosylation with CP4. Mutations 4, 5, and 6 are located in the N-terminal loop (A12 to K34), whereas mutations 47 and 48 are located in the short C-terminal loop (E427 to V439). The exact position of these mutations within the PLY structure is indicated in Table 1 below with reference to the amino acid positions of SEQ ID NO. 1.

TABLE 1
Glycosylation-sites introduced into dPLY
(pGVXN1979) by site-directed mutagenesis.

| | | aa-exchange |
|---|---|---|
| plasmid-ID | mutation-ID | within dPLY |
| pGVXN 1979 | — | — |
| pGVXN 2193 | mut 1 | K4 (→KDQNATK) |
| pGVXN 2196 | mut 4 | Q24 (→KDQNATK) |
| pGVXN 2197 | mut 5 | S27 (→KDQNATK) |
| pGVXN 2198 | mut 6 | E29 (→KDQNATK) |
| pGVXN 2202 | mut 10 | S68 (→KDQNATK) |
| pGVXN 2207 | mut 15 | S109 (→KDQNATK) |
| pGVXN 2208 | mut 16 | L113 (→KDQNATK) |
| pGVXN 2211 | mut 19 | Q140 (→KDQNATK) |
| pGVXN 2212 | mut 20 | P145 (→KDQNATK) |
| pGVXN 2217 | mut 25 | A206 (→KDQNATK) |
| pGVXN 2223 | mut 31 | K271 (→KDQNATK) |
| pGVXN 2224 | mut 32 | K279 (→KDQNATK) |
| pGVXN 2225 | mut 33 | P296 (→KDQNATK) |
| pGVXN 2226 | mut 34 | R301 (→KDQNATK) |
| pGVXN 2227 | mut 35 | G305 (→KDQNATK) |
| pGVXN 2229 | mut 37 | P325 (→KDQNATK) |
| pGVXN 2239 | mut 47 | L431 (→KDQNATK) |
| pGVXN 2240 | mut 48 | E434 (→KDQNATK) |

### Example 2: Molecular Cloning and Synthesis of Expression-Plasmids Encoding an ORF for a Detoxified Pneumolysin (dPLY) with a Hexa-Histidine-Tag (His₆) and a Signal Sequence (PelB-ss) for Periplasmic Translocation

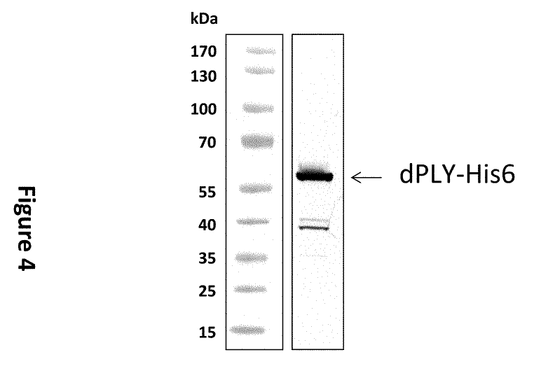

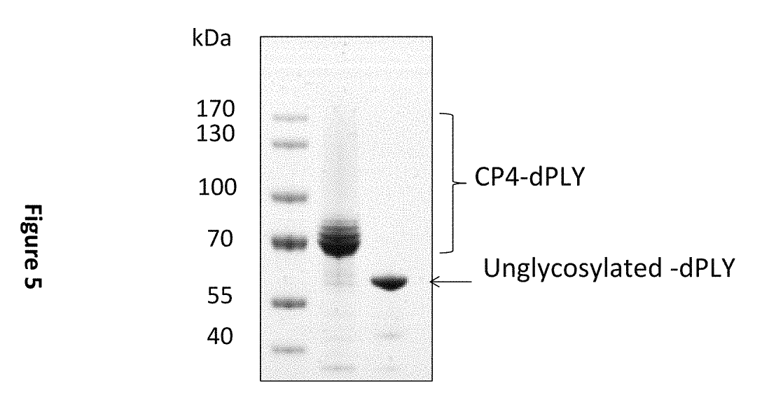

To provide in E. coli cells a carrier protein that harbors a translocation signal for periplasmic expression, a codon-usage optimized (for expression in E. coli) open-reading-frame (ORF) encoding detoxified S. pneumoniae pneumolysin (dPLY) harboring 5 detoxifying mutations (G293V, A370E, C428A, W433E, and L460E) and containing an hexa-histidine-tag (His6) was synthesized and cloned in frame with the ORF of the signal sequence from the pectase lyase 2 precursor of P. carotovorum (PelB-ss). The synthesized ORF (_{PelB-ss}dPLY_{His6}) (SEQ ID NO. 32) flanked by unique restriction sites for NdeI and XmaI was cloned into the NdeI and XmaI-restriction sites pGVXN1184 (pEC415-Kan) resulting in plasmid pGVXN1979. To assess periplasmic secretion E. coli StGVXN4274 (W3110 ΔaraBAD) was transformed with pGVXN1979 and transformants were selected on LB-agar plates containing kanamycin [50 ug/ml] for over-night growth at 37° C. and used to inoculate a liquid LB-medium preculture containing kanamycin [50 ug/ml]. The preculture was used to inoculate a 100 ml TBdev medium main culture supplemented with MgCl₂ [10 mM] and kanamycin [50 ug/ml] to reach an OD_{600 nm} of 0.1. Expression of _{PelB-ss}dPLY_{His6} was induced at an OD_{600 nm} 0.68 with 0.1% arabinose. After o/n (overnight) expression OD_{600 nm} equivalents were harvested and subjected to periplasmic extract enrichment. 2 OD_{600 nm} equivalents of the periplasmic extract (PPE) were loaded onto a SDS-PAGE (4-12% NuPAGE) and analyzed by Westernblotting (see FIG. 4) using an anti-His antibody (Penta-His Antibody, Quiagen).

### Example 3: Syntheses of Expression-Plasmids Encoding an ORF for a Detoxified Pneumolysin (dPLY) with Five Detoxifying Mutations, a Glycosylation-Site, a Hexa-Histidine-Tag (His₆) and a Signal Sequence (PelBss) for Periplasmic Translocation

To provide in E. coli cells a carrier protein that harbors as acceptor a suitable glycosylation-site for periplasmic pglB-dependent protein-glycosylation, codon-usage optimized (for expression in E. coli) open-reading-frames (ORFs) of detoxified S. pneumoniae pneumolysin (dPLY) were altered by site-directed mutagenesis to contain a glycosylation-site (replacing an amino acid with the bacterial N-glycosylation consensus sequon KDQNATK (SEQ ID NO.31), as listed in Table 1; SEQ ID NOs:33-50). The plasmid pGVXN1979 (p_{PeB-ss}dPLY_{His6}) harboring 5 detoxifying mutations (G293V, A370E, C428A, W433E, and L460) was used.

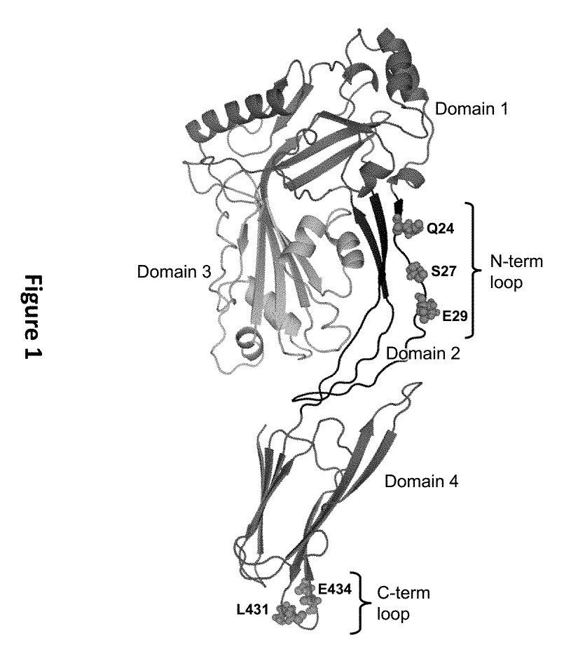

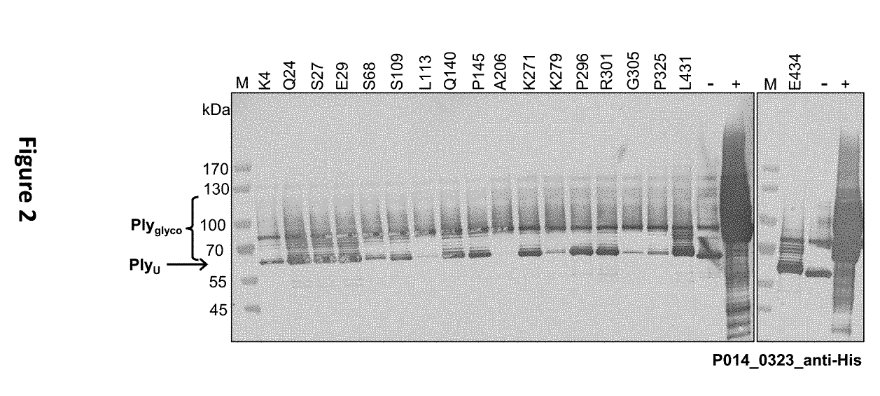

The 18 resulting _{PelB-ss}dPLY^{mut}_{His6} expression plasmids were analyzed for periplasmic expression and glycosylation efficiency of dPLY mutants by PglB with the CP4 as described below (see FIG. 2). The results indicate that the regions covering the N-terminal and C-terminal loop (compare FIG. 1) including the residues Q24, S27, E29, L431 and E434 are representing preferred sites for glycosylation of dPLY by PglB.

### Example 4: Molecular Cloning and Synthesis of Expression-Plasmids Encoding an ORF for a Detoxified Pneumolysin (dPLY) with Six Detoxifying Mutations, a Glycosylation-Site, a Hexa-Histidine-Tag (His₆) and a Signal Sequence (PelB-ss) for Periplasmic Translocation

The plasmid pEC415-plasmid pGVXN1979 harbors an arabinose-inducible expression cassette for the pneumolysin-ORF containing 5 detoxifying mutations (G293V, A370E, C428A, W433E, and L460E) fused NH2-terminally to a PelB signal sequence and COOH-terminally to a hexa-histidine tag (see SEQ ID NO. 32, as provided by Gene Synthesis (sertive for the chemical de-novo synthesis of DNA-sequence of interest), see above)). To provide further detoxifying mutations, a disulfide cross-link was introduced by site-directed mutagenesis into the ORF of pneumolysin, leading to 6 detoxifying mutations, namely T65C, V293C, A370E, C428A, W433E, and L460E. The pneumolysin-ORF containing these six mutations was fused NH2-terminally to a PelB signal sequence and COOH-terminally to a hexa-histidine tag. The resulting plasmid was named pGVXN2369.

Into the pneumolysin-ORF of pGVXN2369 a bacterial N-glycosylation consensus sequon (KDQNATK, SEQ ID NO.31) was introduced by either replacing Q24 (Q24KDQNATK, dPLYₘᵤₜ₄) or E434 (E434KDQNATK, dPLYₘᵤₜ₄₈). The resulting pneumolysin-ORFs contained 6 detoxifying mutations and a glycosylation site and were fused NH2-terminally to a PelB signal sequence and COOH-terminally to a hexa-histidine tag. The resulting plasmids were named pGVXN2400 (p_{PelB-ss}dPLy^{mut4}_{His6}) and pGVXN2401 (p_{PelB-ss}dPLY^{mut48}_{His6}), respectively.

### Example 5: Molecular Cloning of Expression-Plasmids Encoding an ORF for a Detoxified Pneumolysin (dPLY) with a Glycosylation-Site, a Hexa-Histidine-Tag (His₆) and Various Signal Sequences for Periplasmic Translocation

To provide in E. coli cells a carrier protein that harbors as acceptor a suitable glycosylation-site for periplasmic pglB-dependent protein-glycosylation, two variants of a codon-usage optimized (for expression in E. coli) open-reading-frame (ORF) of S. pneumoniae pneumolysin containing six detoxifying mutations (dPLYT65C, G293C, A370E, C428A, W433E, L460E) and containing one glycosylation-site (and an hexa-histidine-tag (His6)) were cloned in frame with the ORF of one of eight signal sequences (ss).

In order to exchange the beta-lactamase gene (bla) on pEC415 (pGVXN315) against an aminoglycoside-3′-o-phosphotransferase gene the method described by Datsenko & Wanner (Datsenke K A & Wanner B L, Proc Natl Acad Sci USA. 2000 Jun. 6; 97(12):6640-5.) was employed.

### In More Detail:

E. coli strain DH5a containing the plasmid pGVXN315 (pEC415-Amp) was transformed with pGVXN837 (modified (contains an aminoglycoside acetyltransferase instead of a beta-lactamase gene) pKD46, containing lambda-red recombinases under araB-promoter (for pKD46 compare Datsenke K A & Wanner B L, 2000) and double-transformants were selected on LB-agar plates containing ampicillin [100 ug/ml] and gentamycin [15 ug/ml] for over-night growth at 30° C. Liquid LB-medium containing ampicillin [100 ug/ml], gentamycin [15 ug/ml] and 0.2% [v/v] arabinose was inoculated with a single colony from DH5a [pGVXN315, pGVXN837] and the culture was grown at 30° C. to an OD_{600 nm} of 0.6 in order to prepare electrocompetent E. coli. Cells were transformed by electroporation with 100 ng of purified PCR-product (encompassing homologous regions needed for recombination and an aminoglycoside-3′-o-phosphotransferase gene obtained by PCR using the oligonucleotides oGVXN2276 and oGVXN2277 and pGVXN73 (pEXT22, see Dykxhoorn D M et al., Gene 177 (1996) 133 136) as a template. Transformed E. coli cells were allowed to recover in liquid SOC at 37° C. for 4 hrs and were plated on LB-agar plates containing kanamycin [50 ug/ml]. E. coli cells in which the bla-gene on the pEC415-based plasmid (pGVXN315) were exchanged against the aminoglycoside-3′-o-phosphotransferase genes were selected for growth at 37° C. on kanamycin and absences of the helper plasmid pGVXN837 and the bla-genes were confirmed by sensitivity towards the antibiotics gentamycin and ampicillin after replica-plating. In addition exchange of the bla-genes with the aminoglycoside-3′-o-phosphotransferase genes on the pEC415 plasmid was confirmed by colony PCR using the oligonucleotide pair oGVXN1672/oGVXN1178. The resulting pEC415-Kan^{R}-plasmids were named pGVXN1184.

Employing site-directed mutagenesis, a NheI-site was introduced into the multiple cloning site (MCS) of pGVXN1184 resulting in pGVXN2555. pGVXN2555 displays within its MCS (among other restriction sites) a NdeI and NheI restriction site which can be used for directional cloning of open-reading frames using the “atg” within the NdeI-site as putative start-codon for translation.

Finally, ORFs for the following signal sequences (ss) were cloned into the NdeI and NheI-restriction sites pGVXN2555 using standard molecular cloning techniques:
i. DsbA-ss (E. coli disulfide oxidoreductase, SEQ ID NO.13), leading to pGVXN2556;
ii. FlgI-ss (S. flexneri flagellar basal body P-ring biosynthesis protein, SEQ ID NO.19), leading to pGVXN2557;
iii. LTIIb-ss, (E. coli heat-labile enterotoxin IIB, B chain, SEQ ID NO.17), leading to pGVXN2558;
iv. MalE-ss (E. coli maltose binding protein, SEQ ID NO.15), leading to pGVXN2559;
v. SipA-ss (S. agalactiae, surface immunogenic protein, SEQ ID NO.70), leading to pGVXN2561;
vi. XynA-ss (B. amyloliquefaciens, Xylanase, SEQ ID NO.18), leading to pGVXN2562
vii. TolB-ss (E. coli translocation protein, SEQ ID NO.20), leading to pGVXN2563;
viii. OmpA-ss (E. coli outer membrane protein, SEQ ID NO.14), leading to pGVXN2564.

By using PCR with pGVXN2400 or pGVXN2401 as a template the S. pneumoniae dPLY^{mut4}_{His6} and dPLY^{mut48}_{His6} gene, respectively, were amplified by PCR and cloned into the NheI and XhoI sites of:
i. pGVXN2556 in frame with the ORF of the DsbA-ss resulting in pGVXN2887 (P_{DsbA-ss}dPLY^{mut4}_{His6}; SEQ ID NO.54) and pGVXN2895 (p_{DsbA-ss}dPLY^{mut48}_{His6}; SEQ ID NO.55);
ii. pGVXN2557 in frame with the ORF of the FlgI-ss resulting in pGVXN2888 (P_{FlgI-ss}dPLY^{mut4}_{His6}; SEQ ID NO.56) and pGVXN2896 (p_{FlgI-ss}dPLY^{mut48}_{His6}; SEQ ID NO.57);
iii. pGVXN2558 in frame with the ORF of the LTIIb-ss resulting in pGVXN2889 (p_{LTIIb-ss}dPLy^{mut4}_{His6}; SEQ ID NO.58) and pGVXN2897 (p_{LTIIb-ss}dPLY^{mut48}_{His6}; SEQ ID NO.59);
iv. pGVXN2559 in frame with the ORF of the MalE-ss resulting in pGVXN2890 (p_{MalE-ss}dPLY^{mut4}_{His6}; SEQ ID NO.60) and pGVXN2898 (p_{MalE-ss}dPLY^{mut48}_{His6}; SEQ ID NO.61);
v. pGVXN2561 in frame with the ORF of the SipA-ss resulting in pGVXN2891 (P_{SipA-ss}dPLY^{mut4}_{His6}; SEQ ID NO.62) and pGVXN2899 (p_{SipA-ss}dPLY^{mut48}_{His6}; SEQ ID NO.63);
vi. pGVXN2562 in frame with the ORF of the XynA-ss resulting in pGVXN2892 (p_{XynA-ss}dPLY^{mut4}_{His6}; SEQ ID NO.64) and pGVXN2900 (p_{XynA-ss}dPLY^{mut48}_{His6}; SEQ ID NO.65);
vii. pGVXN2563 in frame with the ORF of the TolB-ss resulting in pGVXN2893 (p_{TolB-ss}dPLY^{mut4}_{His6}; SEQ ID NO.66) and pGVXN2901 (p_{TolB-ss}dPLY^{mut48}_{His6}; SEQ ID NO.67);
viii. pGVXN2564 in frame with the ORF of the OmpA-ss resulting in pGVXN2894 (p_{OmpA-ss}dPLY^{mut4}_{His6}; SEQ ID NO.68) and pGVXN2902 (p_{OmpA-ss}dPLY^{mut48}_{His6}; SEQ ID NO.69);

Those plasmids can be used to express the either dPLy^{mut4}_{His6} or dPLY^{mut48}_{His6} in the periplasm of E. coli.

Molecular Cloning of pGVXN803:

Using genomic DNA isolated from a Streptococcus pneumoniae type 4 wildtype strain the full-length gene cluster encoding the genes necessary for the synthesis of capsular polysaccharide from serotype 4 (CP4) was amplified by PCR and ligated into the XhoI and AscI sites of the pLAFR-derivate pGVXN725 (pLAFR_J23103-RBS-MCS-term_J23103-RBS-MCS-term). The resulting plasmid was named pGVXN803.

### Example 6: Production and Purification of dPLY-CP4 Bioconjugate

E. coli StGVXN1128 (W3110 ΔwaaL) was co-transformed with the plasmids encoding (a) the Streptococcus pneumoniae capsular polysaccharide type 4 (CP 4) pGVXN803, (b) the S. pneumoniae carrier protein dPLY (detoxified Pneumolysin, carrying the detoxifying mutations: T65C, G293C, A370E, C428A, W433E, L460E), carrying a glycosylation site at position 434 (mut48) and a C-terminal hexa-histidine (His6) affinity tag (pGVXN2401, p_{PelB-ss}dPLY^{mut48}_{His6}), (c) the Campylobacter jejuni oligosaccharyltransferase PglB_{cuo} (pGVXN970) and (d) the UDP-GlcNAc/Glc 4-Epimerase (gne) from Campylobacter jejuni (pGVXN207). Cells were co-transformed by electroporation and grown overnight on selective agar plates supplemented with the four antibiotics tetracycline [20 ug/ml], kanamycine [50 ug/ml], trimethoprim [100 ug/ml] and spectinomycin [80 ug/ml].

Cells were recovered from the agar plates and inoculated into a 1000 ml TBdev preculture supplemented with MgCl₂ [10 mM] and the four antibiotics tetracycline [20 ug/ml], kanamycine [50 ug/ml], trimethoprim [100 ug/ml] and spectinomycin [80 ug/ml] and incubated overnight at 37° C. The preculture was used to inoculate a 20 L Bioreactor containing 7 L TBdev medium supplemented with MgCl₂ [10 mM] to reach an OD_{600 nm} of 0.25. Recombinant polysaccharide was expressed constitutively, while PglB was induced with 1 mM isopropyl-1-D-thiogalactopyranoside (IPTG), and dPLY and Gne were induced with 0.4% arabinose, at an optical density OD_{600 nm} of 30, and the vessel was fed with 3.5 LTB medium (186 ml/h) over night.

After overnight induction, a total of 945,000 ODs were harvested from the vessel by centrifugation and cell pellets were stored at −80° C. From a total of 400,000 ODs (2×200,000 ODs) bioconjugate was extracted by an osmotic shock treatment. A cell equivalent of 200,000 ODs were washed with 0.9% NaCl and collected by centrifugation. The pellet was resuspended in 666 ml ⅓×TBS and to the sample 333 ml resuspension buffer (600 mM Tris, 30 mM EDTA, 75% Sucrose, pH 8.5) was added and the suspension was incubated by stirring in the cold room for 30 min. The suspension was centrifuged for 30 min at 4° C. and 10,000 rpm, supernatant was stored at 4° C. and the pellet was resuspended in the same volume (1000 ml) with osmotic shock buffer (10 mM Tris, pH 8). The suspension was incubated by stirring in the cold room for 30 min and cleared by centrifugation for 30 min at 4° C. and 9,000 rpm. To the supernatant (960 ml) 1M MgCl2 was added to yield a final concentration of 50 mM MgCl2 and it was diluted with 240 ml 5× binding buffer (150 mM Tris-HCl, pH 8.0, 2.5M NaCl, 50 mM Imidazole). This PPE (1250 ml) was filtrated (0.22 um) before loading an IMAC (Immobilized metal affinity chromatography) column (GE Healthcare XK 26/70 with 100 ml equilibrated (lx binding buffer, 30 mM Tris-HCl, pH 8.0, 500 mM NaCl, 10 mM Imidazole) IMAC resin (Tosoh Toyopearl)). The sample was loaded on the IMAC column with a peristaltic pump at RT (room temperature) (flow rate 5 ml/min) and the column was washed with 4 CV (column volume) 1× binding buffer. Bound protein was eluted in 14 ml fractions using a linear gradient (1-50% buffer B) with a length of 15CV (buffer A: 30 mM Tris-HCl, pH 8.0, 50 mM NaCl, buffer B: 30 mM Tris-HCl, pH 8.0, 50 mM NaCl, 1M Imidazole). Eluted fractions were analyzed by SDS-PAGE (4-12% NuPAGE) and Westernblotting using an anti-His antibody (Penta-His Antibody, Quiagen) and fractions containing his-tagged bioconjugate were pooled and diluted with buffer A (30 mM Tris-HCl, pH 7.5) to yield a conductivity of 8.82 mS/cm (mS is Milli-Siemens).

The pooled sample (600 ml) was loaded on an equilibrated 50 ml PallIQ (Ceramic HyperD F) column and the column was washed with 5CV buffer A (30 mM Tris-HCl, pH 7.5). Bound bioconjuage was eluted in 10 ml fractions using a linear gradient (0-50% buffer B) with a length of 20CV (buffer A: 30 mM Tris-HCl, pH 7.5, 50 mM NaCl, buffer B: 30 mM Tris-HCl, pH 7.5, 1000 mM NaCl). Eluted fractions were analyzed by Coomassie-stained (Simply Blue Coomassie) SDS-PAGE (4-12% NuPAGE) and Westernblotting using an anti-CP4 antibody (Pneumococcal antisera Type 4 (rabbit), Serum Statens Institute) and fractions containing PLY-CP4 bioconjugate were pooled and diluted with buffer A (30 mM Tris-HCl, pH 7.5) to yield a conductivity of 8.69 mS/cm.

The pooled sample (480 ml) was loaded on an equilibrated 20 ml SourceQ column and the column was washed with 5CV buffer A (30 mM Tris-HCl, pH 7.5). Bound bioconjuage was eluted in 5 ml fractions using a first linear gradient (0-15.5% buffer B) with a length of 10CV followed by a second linear gradient (15.5-18% buffer B) with a length of 2CV and by a third linear gradient (18-50% buffer B) with a length of 8CV (buffer A: 30 mM Tris-HCl, pH 7.5, 50 mM NaCl, buffer B: 30 mM Tris-HCl, pH 7.5, 1000 mM NaCl). Eluted fractions were analyzed by Coomassie-stained (Simply Blue Coomassie) SDS-PAGE (4-12% NuPAGE) and Westernblotting using an anti-CP4 antibody (Pneumococcal antisera Type 4 (rabbit), Serum Statens Institute) and fractions containing PLY-CP4 bioconjugate were pooled (to yield 50 ml) and adjusted to Hydrophobic Interaction Chromatography (HIC) (HIC PPG-600M) loading conditions by adding dropwise Ammonium sulfate to reach a final concentration of 1 M Ammonium sulfate within the load sample. 2 ml PPG-600M resin was equilibrated with 10CV buffer A (1.0M Ammonium sulfate in 5 mM Na-Phosphate pH 7.2) and mixed with the load sample and incubated for 10 min at RT on a rotating wheel. The suspension was poured into a column device and the flow through was collected. The column was washed 4 times with 5CV buffer A (1.0M Ammonium sulfate in 5 mM Na-Phosphate pH 7.2). Each wash was collected as 10 ml fractions. Bound protein was eluted with 5CV ddH₂O. Flow-through, wash-fractions and eluate were analyzed by Coomassie-stained (Simply Blue Coomassie) SDS-PAGE (4-12% NuPAGE) and Westernblotting using an anti-His antibody (Penta-His Antibody, Quiagen) and fractions (i.e. flow-through and wash1 and wash2 fractions) containing the his-tagged bioconjugate were pooled and concentrated using a Amicon Centrifugal Device (10 k MWCO) to a volume of 0.5 ml. Pooled sample was loaded onto a Superdex 200 (10/300, volume=24 ml) column and 0.5 ml fractions were collected using 1×TBS (Tris-buffered saline). Collected fractions were analyzed by Coomassie-stained (Simply Blue Coomassie) SDS-PAGE (4-12% NuPAGE) and SEC-fractions containing CP4-dPLY bioconjugate were pooled, sterile filtrated (Costar Centrifuge Tube filter 0.22 um) and protein-concentration was determined to be 0.404 mg/ml.

Within this pooled sample the endotoxin level was analyzed and determined to be 24.2 EU/ml.

2.5 ug of the purified final CP4-dPLY sample was analyzed side-by-side with 1 ug non-glycosylated dPLY (uPLY) by Coomassie-stained (Simply Blue Coomassie) SDS-PAGE (4-12% NuPAGE), FIG. 5. The result indicates that CP4-dPLY could be purified to high homogeneity and was separated well from non-glycosylated dPLY and inpurities during the described purification procedure.

### Example 7: Production and Purification of dPLY-CP33F Bioconjugate

E. coli StGVXN9329 (W3110 wca::33F_{wt}; waaL::pglB_{cuo}; ECA::33F_{eng}; rfb::cat) was co-transformed with (a) a plasmid encoding the S. pneumoniae carrier protein dPLY (detoxified Pneumolysin, carrying the detoxifying mutations: T65C, G293C, A370E, C428A, W433E, L460E), carrying a glycosylation site at position 434 (mut48) and a C-terminal hexa-histidine (His6) affinity tag (pGVXN2901, p_{TolB-ss}dPLY^{mut48}_{His6}), and (b) a plasmid (pGVXN1883) carrying the gene for a positive activator of the colanic acid locus (rcsA). Cells were co-transformed by electroporation and grown overnight on selective agar plates supplemented with kanamycin [50 ug/ml], and spectinomycin [80 ug/ml].

Cells recovered from the agar plates were inoculated into a 50 ml TBdev preculture supplemented with MgCl₂ [10 mM] and the two antibiotics kanamycin [50 ug/ml] and spectinomycin [80 ug/ml] and incubated overnight at 30° C. The preculture was used to inoculate a shake flask containing 2 L TBdev medium supplemented with MgCl₂ [10 mM], kanamycin [50 ug/ml], spectinomycin [80 ug/ml], and 0.1 mM isopropyl-β-D-thiogalactopyranoside (IPTG) to reach an OD_{600 nm} of 0.25. Recombinant polysaccharide and PglB was expressed upon addition of IPTG, while dPLY was induced with 0.5% arabinose after 8 hrs. The same time, 0.9 mM IPTG was added for a second time and the culture was shifted to 37° C. for o/n (overnight) induction.

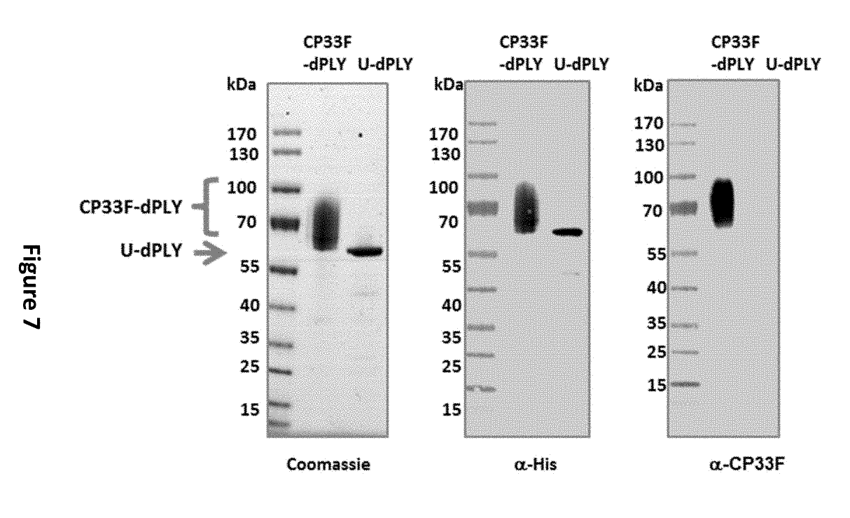

After overnight induction, a total of 15,400 ODs were harvested by centrifugation and washed with 0.9% NaCl. Cell pellet was resuspended in 52 ml TBS and to the sample 26 ml resuspension buffer (600 mM Tris, 30 mM EDTA, 75% Sucrose, pH 8) was added and the suspension was incubated by stirring in the cold room for 30 min. The suspension was centrifuged for 30 min at 4° C. and 8,000 rpm, supernatant was stored at 4° C. and the pellet was resuspended in the same volume (78 ml) with osmotic shock buffer (10 mM Tris, pH 8). The suspension was incubated by stirring in the cold room for 30 min and cleared by centrifugation for 30 min at 4° C. and 8,000 rpm. The supernatant was filtrated (0.45 um) and 1M MgCl2 was added to yield a final concentration of 50 mM MgCl2 and it was diluted with 5× binding buffer (150 mM Tris-HCl, pH 8.0, 2.5M NaCl, 50 mM Imidazole) to yield a final concentration of 1× binding buffer within the sample. This PPE (80 ml) was been filtrated (0.22 um) before loading on the IMAC (Immobilized metal affinity chromatography) column (Millipore VL 11×250 with 20 ml equilibrated (1× binding buffer, 30 mM Tris-HCl, pH 8.0, 500 mM NaCl, 10 mM Imidazole) IMAC resin (Tosoh Toyopearl). The sample was loaded on the IMAC column with a peristaltic pump at RT (flow rate 5 ml/min) and the column was washed with 5 CV 1× binding buffer. Bound protein was eluted in 5 ml fractions using a linear gradient (1-50% buffer B) with a length of 7.5CV (buffer A: 30 mM Tris-HCl, pH 8.0, 50 mM NaCl, buffer B: 30 mM Tris-HCl, pH 8.0, 50 mM NaCl, 1M Imidazole). Eluted fractions were analyzed by Coomassie-stained (Simply Blue Coomassie) SDS-PAGE (4-12% NuPAGE) and Westernblotting using an anti-His antibody (Penta-His Antibody, Quiagen) and fractions containing his-tagged bioconjugate were pooled (45 ml) and diluted with 105 ml buffer A (30 mM Bis-Tris, pH 6.0). The pooled sample (150 ml) was loaded on an equilibrated 20 ml SourceQ column and the column was washed with 3CV buffer A (30 mM Bis-Tris, pH 6.0). Bound bioconjuage was eluted in 5 ml fractions using linear gradient (0-50% buffer B) with a length of 30CV (buffer A: 30 mM Tris-Bis, pH 6.0, buffer B: 30 mM Bis-Tris, pH 6.0, 1000 mM NaCl). Eluted fractions were analyzed by Coomassie-stained (Simply Blue Coomassie) SDS-PAGE (4-12% NuPAGE) and Westernblotting using an anti-CP33F antibody (Pneumo Factor serum 33b (rabbit), Serum Statens Institute) and an anti-His antibody (Penta-His Antibody, Quiagen), respectively. Fractions containing CP33F-dPLY bioconjugate were pooled (to yield 10 ml) and concentrated using a Amicon Centrifugal Device (10 k MWCO) to a volume of 0.5 ml. The pooled sample was loaded onto a Superdex 200 (10/300, volume=24 ml) column and 0.5 ml fractions were collected using 1×TBS, pH7.4. Collected fractions were analyzed by Coomassie-stained (Simply Blue Coomassie) SDS-PAGE (4-12% NuPAGE) and SEC-fractions containing CP33F-dPLY bioconjugate were pooled and protein-concentration was determined to be 0.158 mg/ml. 2.5 ug of the purified final CP33F-dPLY sample was analyzed side-by-side with 1.5 ug non-glycosylated dPLY (uPLY) by Coomassie-stained (Simply Blue Coomassie) SDS-PAGE (4-12% NuPAGE) and 1 ug of the purified final CP33F-dPLY sample was analyzed side-by-side with 0.45 ug non-glycosylated dPLY (uPLY) by Westernblotting using anti-CP33F antibody (Pneumo Factor serum 33b (rabbit), Serum Statens Institute) and an anti-His antibody (Penta-His Antibody, Quiagen), respectively, see FIG. 7. These results indicate that dPLY gets efficiently glycosylated with CP33F and could be purified to high homogeneity by the procedure described above. Non-glycosylated dPLY could neither be detected by Coomassie-stain of the SDS-PAGE nor by anti-His Westernblotting.

### Example 8: ELISA Procedures to Measure the IgG Response Against CP4, EPA and Ply in Guinea Pigs

CP4

In Microtiter 96-well plates (MAXISORP™, Nunc, Thermo Scientific), the raw A was coated with 100 μl per well of goat anti-guinea pig IgG polyclonal antibodies (Jackson 106-005-003 at 2.4 mg/ml) at 2 μg/ml in PBS buffer. The raws B to H were coated with 100 μl per well of CP4 (PS04P130 at 2 mg/ml) at 5 μg/ml in PBS buffer. After incubation 2 hours at 37° C., the plates were washed three times with NaCl 0.09% TWEEN™ 20 0.05%. After washing, serial two fold dilutions (in PBS TWEEN™ 20 0.05%) of purified guinea pig IgG (Jackson 006-000-003 at 0.25 μg/ml) used as a standard reference to dose the corresponding IgG in sera were added in raw A. Then serial two fold dilutions of tested sera were added into raws B to H in PBS-TWEEN™ 20 0.05%. The plates were incubated for 30 minutes at room temperature under shaking. After washing, peroxidase-conjugated goat anti-guinea IgG antibodies (Jackson 106-035-003) were added at a dilution of 1/2000 final (100 μl per well) for 30 min at room temperature under shaking. Plates were washed as above and the solution of revelation [4 mg of O-Phenylenediamine dihydrochloride (OPDA) and 5 μl of H₂O₂ in 10 ml of citrate 0.1M pH 4.5 buffer] was added to each well (100 μl/well) for 15 min in the darkness. The reaction was stopped by addition of 50 μl of HCl 1N and the optical density (OD) was read at 490 nm (620 nm for the reference filter). The individual IgG concentrations (expressed as μg/ml) were calculated by the 4-parameter method using the Soft Max Pro software.

EPA

In Microtiter 96-well plates (MAXISORP™, Nunc, Thermo Scientific), the raw A was coated with 100 μl per well of goat anti-guinea pig IgG polyclonal antibodies (Jackson 106-005-003 at 2.4 mg/ml) at 2 μg/ml in PBS buffer. The raws B to H were coated with 100 μl per well of EPA (EPA E-5_6 at 1.996 mg/ml) at 2 μg/ml in PBS buffer. After overnight incubation at 4° C., the plates were washed three times with NaCl 0.09% TWEEN™ 20 0.05%. After washing, serial two fold dilutions (in PBS TWEEN™ 20 0.05%) of purified guinea pig IgG (Jackson 006-000-003 at 0.25 μg/ml) used as a standard reference to dose the corresponding IgG in sera were added in raw A. Then serial two fold dilutions of tested sera were added into raws B to H in PBS-TWEEN™ 20 0.05%. The plates were incubated for 30 minutes at room temperature under shaking. After washing, peroxidase-conjugated goat anti-guinea IgG antibodies (Jackson 106-035-003) were added at a dilution of 1/2000 final (100 μl per well) for 30 min at room temperature under shaking. Plates were washed as above and the solution of revelation [4 mg of OPDA and 5 μl of H₂O₂ in 10 ml of citrate 0.1M pH 4.5 buffer] was added to each well (100 μl/well) for 15 min in the darkness. The reaction was stopped by addition of 50 μl of HCl 1N and the OD was read at 490 nm (620 nm for the reference filter). The individual IgG concentrations (expressed as μg/ml) were calculated by the 4-parameter method using the Soft Max Pro software.

Ply

In Microtiter 96-well plates (MAXISORP™, Nunc, Thermo Scientific), the raw A was coated with 100 μl per well of goat anti-guinea pig IgG polyclonal antibodies (Jackson 106-005-003 at 2.4 mg/ml) at 2 μg/ml in PBS buffer. The raws B to H were coated with 100 μl per well of Ply (EPly 07A at 5.98 mg/ml) at 8 μg/ml in PBS buffer. After incubation 2 hours at 37° C., the plates were washed three times with NaCl 0.09% TWEEN™ 20 0.05%. After washing, serial two fold dilutions (in PBS TWEEN™ 20 0.05%) of purified guinea pig IgG (Jackson 006-000-003 at 0.25 μg/ml) used as a standard reference to dose the corresponding IgG in sera were added in raw A. Then serial two fold dilutions of tested sera were added into raws B to H in PBS-TWEEN™ 20 0.05%. The plates were incubated for 30 minutes at room temperature under shaking. After washing, peroxidase-conjugated goat anti-guinea IgG antibodies (Jackson 106-035-003) were added at a dilution of 1/2000 final (100 μl per well) for 30 min at room temperature under shaking. Plates were washed as above and the solution of revelation [4 mg of OPDA and 5 μl of H₂O₂ in 10 ml of citrate 0.1M pH 4.5 buffer] was added to each well (100 μl/well) for 15 min in the darkness. The reaction was stopped by addition of 50 μl of HCl 1N and the OD was read at 490 nm (620 nm for the reference filter). The individual IgG concentrations (expressed as μg/ml) were calculated by the 4-parameter method using the Soft Max Pro software.

### Example 9: Opsonophagocytosis Assay Procedure

The functionality of anti-pneumococcal polysaccharide 4 antibodies was evaluated using a serotype specific opsonophagocytosis assay. The serum samples were heated at 56° C. (to inactivate their complement content) and then two-fold serially diluted in 25 μl HBSS-BSA (Hanks' balanced salt solution-bovine serum albumin) 3% in a 96 well round bottom plate. Twenty-five μl of a mixture of activated HL60 cells (promyelocytic human HL-60 cells were differentiated into neutrophils by using N,N dimethylformamide), pneumococci and baby rabbit complement were added in a 4/2/1 ratio. The plates were incubated for 2 hours at 37° C. (under shaking to promote the phagocytosis process). A 20 μl aliquot of each well was then transferred into the corresponding well of a 96-well flat bottom microplate. Fifty μl of Todd-Hewitt broth-0.9% agar were added twice into each well. After overnight incubation at 37° C., the pneumococcal colonies were counted using an automated image analysis system (Axiovision). The mean number of colony forming units of eight wells containing bacteria without any serum was determined and used for the calculation of the killing activity of each serum sample. The bactericidal titers were expressed as the reciprocal dilution of serum inducing 50% of killing.

### Example 10: Hemolysis Inhibition Assay

One hundred (100) μl of chloroform treated (to remove serum cholesterol) sera were added to a 96-well U-bottom plate and serially diluted (two-fold dilutions) in the assay buffer (PBS containing 0.155% dithiothreitol and 1% BSA, diluted 10-fold in Tris 15 mM NaCl 150 mM pH 7.5). To fifty (50) μl was added fifty (50) μl of native Ply at 4HU (hemolytic units) (previously determined in the hemolytic assay) and incubated for 15 min at 37° C. A sheep red blood cells suspension (100 μl) was added at the concentration of 1% to each well. The plates were incubated for 30 min at 37° C. and then centrifuged at 900 rpm for 10 minutes at 4° C. 150 μl of the supernatant were transferred in micro plate wells and the OD was read at 405 nm using a spectrophotometer. The hemolytic activity was calculated as the dilution of each sample resulting in 50% of hemolysis inhibition.

### Example 11: Preclinical Evaluation of CP4-dPLY Bioconjugate

In order to evaluate dPLY as a carrier protein for S. pneumoniae bioconjugates CP4-dPLY was used to immunize guinea pigs and compared to an established carrier protein, namely EPA (exotoxin A of P. Aeruginosa) conjugated to the capsular polysaccharide from S. pneumoniae type 4 (CP4-EPA).

In more detail, 5 to 8 weeks old female Dunklin-Hartley guinea pigs (n=12/group) were immunized on days 0, 14 and 28 intramuscularly with either 0.1 ug CP4-EPA (group 1, CP4-EPA, n=12), 1 ug CP4-EPA (group 2, n=12), 0.1 ug CP4-dPLY (group 3, n=12) or 1 ug CP4-dPLY (group 4, n=12), respectively. Bioconjugate vaccines were adjuvanted with Alhydrogel to yield a final concentration of 0.06% Al³⁺. As control 4 animals received only TBS plus 0.06% Al³⁺ (group 5, n=4). Animals were bled on the lateral tarsal vein on days 0 (pre), and 42 (post).

Serological assays were applied to monitor the immunogenicity and functionality of the antibodies within the sera induced in the course of the immunization study.

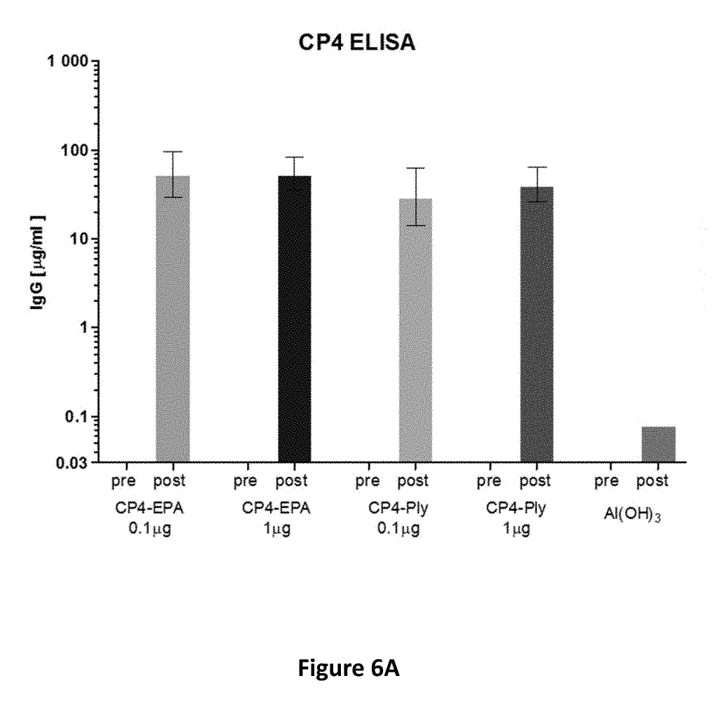

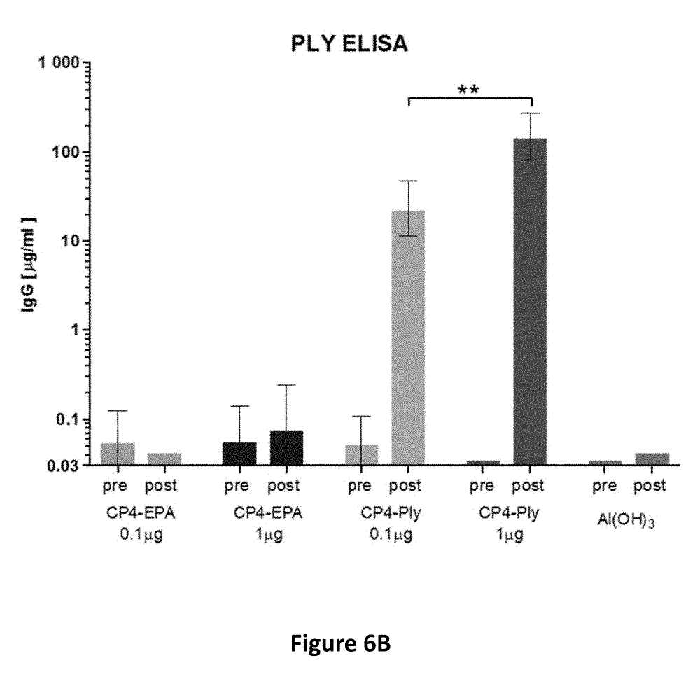

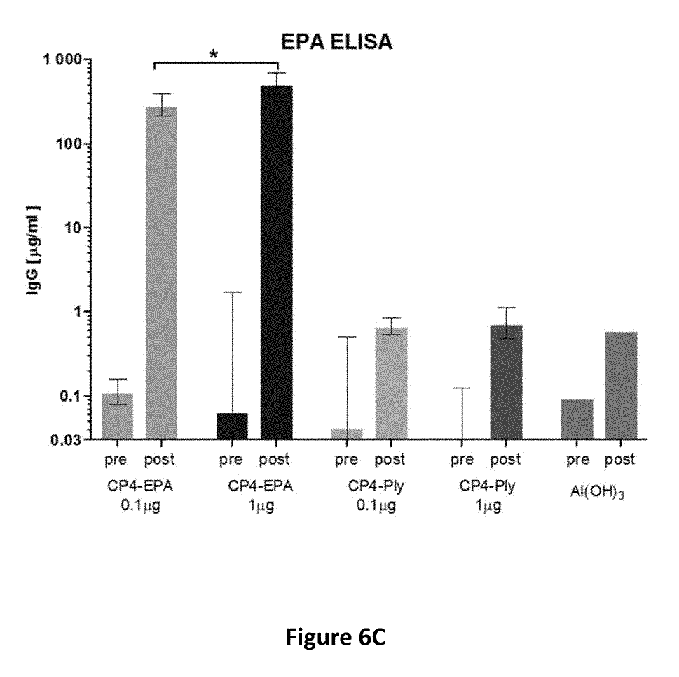

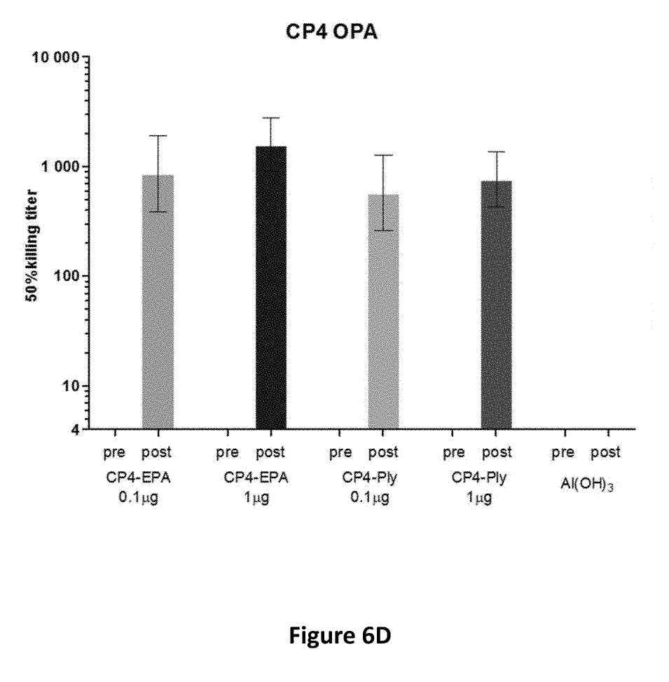

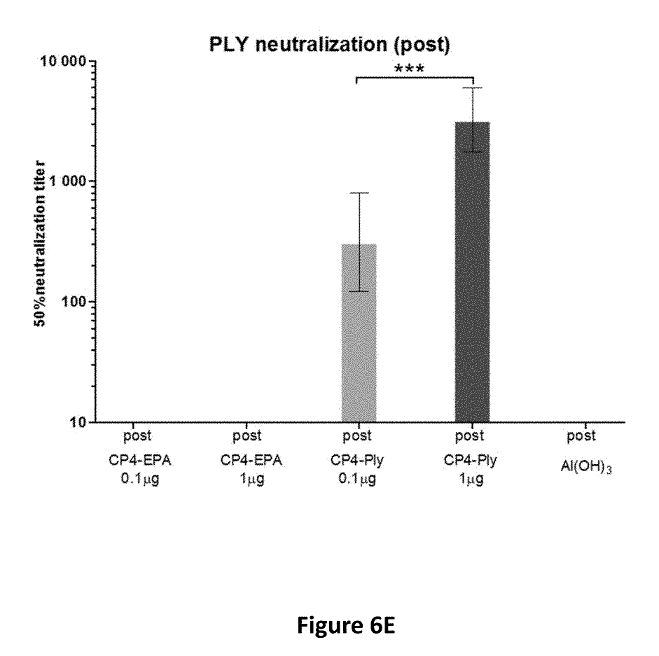

Anti-IgG levels in sera against CP4 (FIG. 6A), dPLY (FIG. 6B), and EPA (FIG. 6C) were tested in ELISA (using the methods described above) in pre and post sera of all groups. Both bioconjugates, CP4-EPA and CP4-dPLY, were immunogenic and induced after three injections a similar level of IgG responses against CP4 without significant differences. Furthermore, post-immunization sera from groups 1, 2, 3, and 4 were equally efficient in mediating opsonophagocytotic killing of pneumococci serotype 4 by activated promyelocytic HL-60 cells (using the method described above) (FIG. 6D) indicating that dPLY and EPA are equally efficient carriers for CP4 in guinea pigs Anti-dPLY IgG responses were detected in post-sera from animals who received CP4-dPLY (FIG. 6B). The anti-dPLY IgG level was higher in the group that received more bioconjugate and hence more carrier protein (compare post-sera from group 3 (0.1 ug CP4-dPLY) and group 4 (1 ug CP4-dPLY) in FIG. 6B). Accordingly high anti-EPA IgG levels were detected in post-sera from animals who received CP4-EPA (FIG. 6C).

The functionality of the bioconjugate-induced anti-dPLY IgG was assayed in a hemolysis inhibition assay (using the method described above). Sera from animals immunized with CP4-dPLY efficiently inhibited hemolysis of red blood cells by native pneumolysin (FIG. 6E) showing that functional anti-dPLY IgGs were induced.

In conclusion, CP4 conjugated to dPLY induced comparable CP4-specific OPA responses compared to CP4-EPA induced neutralizing antibodies specific for pneumolysin. This allows the design of new conjugate vaccines with potentially broader coverage by including different protein antigens from S. pneumoniae conjugated to different CPs.

### Example 12 Production and Purification of dPLY-CP12F Bioconjugate

E. coli StGVXN9876 (W3110 waaL::pglB_{N311V-K482R-D483H-A669V}; ECA::gne; rfb::(wzg-fnlC)_{S.pneumoniae12F} was co-transformed with the plasmids encoding the S. pneumoniae carrier protein dPLY (detoxified Pneumolysin, carrying the detoxifying mutations: T65C, V293C, A370E, C428A, W433E, L460E), carrying a glycosylation site at position 434 (mut48) and a C-terminal hexa-histidine (His6) affinity tag (pGVXN2401, p_{PelB-ss}dPLY^{mut48}_{His6}), and a pLAFR-plasmid (pGVXN3177) carrying the S. pneumoniae 12F genes wciJ-wcxB-wzy-wcxD-wcxE-wzxF by electroporation and grown overnight on selective agar plates supplemented with the kanamycine [50 ug/ml], and tetracycline [20 ug/ml].

Cells were inoculated into a 5 ml TBdev preculture supplemented with MgCl₂ [10 mM] and the two antibiotics kanamycine [50 ug/ml], and tetracycline [20 ug/ml] and incubated overnight at 30° C. The preculture was used to inoculate a shake flask containing 50 mL TBdev medium supplemented with MgCl₂ [10 mM], kanamycine [50 ug/ml], and tetracycline [20 ug/ml], to reach an OD_{600 nm} of 0.1 and incubated at 30° C. Recombinant dPLY was induced with 0.4% arabinose at an OD_{600 nm} of 0.63 and at an OD_{600 nm} of 1.87 the PglB was expressed upon addition of 1 mM IPTG, and the culture was shifted to 37° C. for o/n induction.

After overnight induction, a total of 100 ODs were harvested by centrifugation and washed with 0.9% NaCl and the periplasmic content of the cells was extracted as periplasmic extract (PPE).

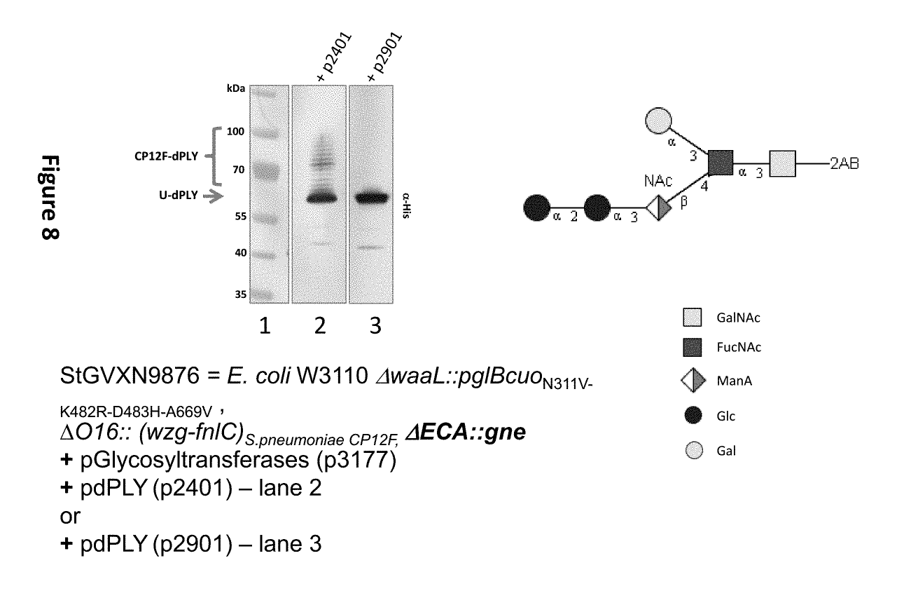

His-tagged protein and bioconjugate was enriched from the PPE by IMAC (Immobilized metal affinity chromatography) and 1 OD equivalent of the eluted fraction was analyzed by Westernblotting using an anti-His antibody (Penta-His Antibody, Quiagen), respectively, see FIG. 8.

The present disclosure is not to be limited in scope by the specific embodiments described herein. Indeed, various modifications of the subject matter provided herein, in addition to those described, will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

1-48. (canceled)

49. A modified pneumolysin protein having an amino acid sequence of SEQ ID NO. 1 or an amino acid sequence at least 80%, 85%, 90%, 92%, 95%, 96%, 97%, 98% or 99% identical to SEQ ID NO. 1, modified in that the amino acid sequence comprises one or more consensus sequence(s) selected from: D/E-X-N-Z-S/T (SEQ ID NO. 28) and K-D/E-X-N-Z-S/T-K (SEQ ID NO. 30), wherein X and Z are independently any amino acid apart from proline.

50. The modified pneumolysin protein of claim 49, wherein one or more amino acids (e.g. 1-7 amino acids, e.g. one amino acid) of the amino acid sequence of SEQ ID NO. 1 or an amino acid sequence at least 80%, 85%, 90%, 92%, 95%, 96%, 97%, 98% or 99% identical to SEQ ID NO. 1 have been substituted by a D/E-X-N-Z-S/T (SEQ ID NO. 28) or K-D/E-X-N-Z-S/T-K (SEQ ID NO. 30) consensus sequence.

51. The modified pneumolysin protein of claim 49, wherein a consensus sequence selected from D/E-X-N-Z-S/T (SEQ ID NO. 28) and K-D/E-X-N-Z-S/T-K (SEQ ID NO. 30) is located at a position within the long N terminal surface loop or the short C terminal loop of SEQ ID NO. 1 or an equivalent position within an amino acid sequence at least 80%, 85%, 90%, 92%, 95%, 96%, 97%, 98% or 99% identical to SEQ ID NO. 1.

52. The modified pneumolysin protein of claim 49, wherein a consensus sequence selected from D/E-X-N-Z-S/T (SEQ ID NO. 28) and K-D/E-X-N-Z-S/T-K (SEQ ID NO. 30) has been added at, or substituted for, one or more amino acids, between amino acid residues 22-57 of SEQ ID NO. 1 or at an equivalent position within an amino acid sequence at least 80%, 85%, 90%, 92%, 95%, 96%, 97%, 98% or 99% identical to SEQ ID NO. 1.

53. The modified pneumolysin protein of claim 49, wherein a consensus sequence selected from D/E-X-N-Z-S/T (SEQ ID NO. 28) and K-D/E-X-N-Z-S/T-K (SEQ ID NO. 30) has been added at, or substituted for, one or more amino acids, between amino acid residues 360-470 of SEQ ID NO. 1 or at an equivalent position within an amino acid sequence at least 80%, 85%, 90%, 92%, 95%, 96%, 97%, 98% or 99% identical to SEQ ID NO. 1.

54. The modified pneumolysin protein of claim 49, comprising a single consensus sequence selected from D/E-X-N-Z-S/T (SEQ ID NO. 28) and K-D/E-X-N-Z-S/T-K (SEQ ID NO. 30).

55. The modified pneumolysin protein of claim 54, wherein the consensus sequence K-D/E-X-N-Z-S/T-K (SEQ ID NO. 30) has been substituted for amino acid residue 434 in SEQ ID NO. 1, or substituted in an amino acid sequence at least 80%, 85%, 90%, 92%, 95%, 96%, 97%, 98% or 99% identical to SEQ ID NO. 1 at an amino acid position equivalent to amino acid residue 434 in SEQ ID NO. 1.

56. The modified pneumolysin protein of claim 49, wherein the amino acid sequence further comprises a signal sequence which is capable of directing the pneumolysin protein to the periplasm of a host cell.

57. The modified pneumolysin protein of claim 49, wherein said signal sequence being selected from SEQ ID NO. 13-20.

58. The modified pneumolysin protein of claim 49, wherein said modified pneumolysin protein having an amino acid sequence at least 97%, 98%, 99% or 100% identical to SEQ ID NO. 21 or SEQ ID NO. 22.

59. A modified pneumolysin protein comprising an amino acid sequence which is at least 80%, 85%, 90%, 92%, 95%, 97%, 98%, 99% or 100% identical to a sequence selected from SEQ ID NOs: 3-10, said amino acid sequence comprising a D/E-X-N-Z-S/T (SEQ ID NO. 28) or K-D/E-X-N-Z-S/T-K (SEQ ID NO. 30) consensus sequence wherein X and Z are independently any amino acid apart from proline and at least one amino acid substitution selected from G293 to C, T65 to C, C428 to A, A370 to E, W433 to E and L460 to E.

60. A conjugate comprising a modified pneumolysin protein of claim 49, wherein the modified pneumolysin protein is linked to an antigen.

61. The conjugate according to claim 60, wherein the modified pneumolysin protein is covalently linked to an antigen through a chemical linkage obtainable using a chemical conjugation method, wherein the chemical conjugation method is selected from a group consisting of carbodiimide chemistry, reductive animation, cyanylation chemistry, maleimide chemistry, hydrazide chemistry, ester chemistry, and N-hydroysuccinimide chemistry either directly or via a linker.

62. The conjugate of claim 60, wherein the antigen is linked to an amino acid on the modified pneumolysin protein selected from asparagine, aspartic acid, glutamic acid, lysine, cysteine, tyrosine, histidine, arginine or tryptophan.

63. The conjugate of claim 60, wherein the saccharide is a bacterial capsular saccharide, selected from a Streptococcus pneumoniae serotype 1, 2, 3, 4, 5, 6A, 6B, 7A, 7B, 7C, 8, 9A, 9L, 9N, 9V, 10A, 10B, 10C, 10F, 11A, 11B, 11C, 11D, 11F, 12A, 12B, 12F, 13, 14, 15A, 15B, 15C, 15F, 16A, 16F, 17A, 17F, 18A, 18B, 18C, 18F, 19A, 19B, 19C, 19F, 20, 21, 22A, 22F, 23A, 23B, 23F, 24A, 24B, 24F, 25A, 25F, 26, 27, 28A, 28F, 29, 31, 32A, 32F, 33A, 33B, 33C, 33D, 33F, 34, 35A, 35B, 35C, 35D, 35F, 36, 37, 38, 39, 40, 41A, 41F, 42, 43, 44, 45, 46, 47A, 47F or 48 capsular saccharide.

64. The conjugate of claim 60, wherein the antigen is a hybrid oligosaccharide or polysaccharide having a structure:
(B)ₙ-A→
wherein A is an oligosaccharide repeat unit containing at least 2, 3, 4, 5, 6, 7 or 8 monosaccharides, with a hexose monosaccharide derivative at the reducing end (indicated by arrow);
wherein B is an oligosaccharide repeat unit containing at least 2, 3, 4, 5, 6, 7 or 8 monosaccharides;
wherein A and B are different oligosaccharide repeat units; and
wherein n is at least 2, 3, 4, S, 6, 7, 8, 9, 10, 11, 12, 13, 14, 15, 16, 17, 18, 19, or at least 20, wherein the hexose monosaccharide at the reducing end of the repeat is selected from the group consisting of glucose, galactose, rhamnose, arabinotol, fucose and mannose.

65. A polynucleotide encoding the modified pneumolysin protein of claim 49.

66. A vector comprising the polynucleotide of claim 65.

67. A host cell comprising:
i) one or more nucleic acids that encode glycosyltransferase(s);
ii) a nucleic acid that encodes an oligosaccharyl transferase; and
iii) a nucleic acid that encodes a modified pneumolysin protein according to claim 49.

68. The host cell of claim 67, further comprising a nucleic acid that encodes a polymerase.

69. The host cell of claim 67, wherein said host cell comprises (a) a glycosyltransferase that assembles a hexose monosaccharide derivative onto undecaprenyl pyrophosphate (Und-PP) and (b) one or more glycosyltransferases capable of adding a monosaccharide to the hexose monosaccharide derivative assembled on Und-PP.

70. The host cell of any one of claim 67, wherein said hexose monosaccharide derivative is any monosaccharide in which C-2 position is modified with an acetamido group such as N-acetylglucosamine (GlcNAc), N-acetylgalactoseamine (GalNAc), 2,4-Diacetamido-2,4,6-trideoxyhexose (DATDH), N-acetylfucoseamine (FucNAc), or N-acetylquinovosamine (QuiNAc).

71. A method of producing a bioconjugate that comprises a modified pneumolysin protein linked to a saccharide, said method comprising (i) culturing the host cell of claim 69 under conditions suitable for the production of proteins and (ii) isolating the bioconjugate.

72. An immunogenic composition comprising the modified pneumolysin protein of claim 49.

73. A method for the treatment or prevention of Streptococcus pneumoniae infection in a subject in need thereof comprising administering to said subject a therapeutically effective amount of the modified pneumolysin protein of claim 49.

74. A method of inducing an immune response to Streptococcus pneumoniae in a subject, the method comprising administering a therapeutically or prophylactically effective amount of the modified pneumolysin protein of claim 49.
